(19) United States
(12) Patent Application Publication
(10) Pub. No.: US 2022/0388985 A1
(43) Pub. Date: Dec. 8, 2022

(54) TARGETED PROTEIN DEGRADATION OF PARP14 FOR USE IN THERAPY

(71) Applicant: Ribon Therapeutics, Inc., Cambridge, MA (US)

(72) Inventors: Laurie B. Schenkel, Somerville, MA (US); Melissa Marie Vasbinder, Newton, MA (US); Kevin Wayne Kuntz, Woburn, MA (US); Kerren Kalai Swinger, Lexington, MA (US); Timothy J.N. Wigle, Cambridge, MA (US)

(21) Appl. No.: 17/619,459

(22) PCT Filed: Jun. 18, 2020
(86) PCT No.: PCT/US2020/038377

§ 371 Date: Dec. 15, 2021

Related U.S. Application Data
(60) Provisional application No. 62/863,357, filed on Jun. 19, 2019.

(51) Int. Cl. C07D 401/14; C07D 417/14; C07D 405/14

(52) U.S. Cl. CPC C07D 401/14; C07D 417/14; C07D 405/14

(57) ABSTRACT

The present invention relates to quinazolinones and related compounds which degrade PARP14 and are useful, for example, in the treatment of cancer and inflammatory diseases.

### FIELD OF THE INVENTION

The present invention relates to quinazolinones and related compounds which cause intracellular proteolysis of PARP14 and are useful in the treatment of cancer and inflammatory diseases.

### BACKGROUND OF THE INVENTION

Poly(ADP-ribose) polymerases (PARPs) are members of a family of seventeen enzymes that regulate fundamental cellular processes including gene expression, protein degradation, and multiple cellular stress responses (Vyas S, et al. Nat Rev Cancer. 2014 Jun. 5; 14(7):502-509). The ability of cancer cells to survive under stress is a fundamental cancer mechanism and an emerging approach for novel therapeutics. One member of the PARP family, PARP1, has already been shown to be an effective cancer target in connection to cellular stress induced by DNA damage, either induced by genetic mutation or with cytotoxic chemotherapy, with three approved drugs in the clinic and several others in late stage development (Ohmoto A, et al. OncoTargets and Therapy. 2017; Volume 10:5195).

The seventeen members of the PARP family were identified in the human genome based on the homology within their catalytic domains (Vyas S, et al. Nat Commun. 2013 Aug. 7; 4:2240). However, their catalytic activities fall into 3 different categories. The majority of PARP family members catalyze the transfer of mono-ADP-ribose units onto their substrates (monoPARPs), while others (PARP1, PARP2, TNKS, TNKS2) catalyze the transfer of poly-ADP-ribose units onto substrates (polyPARPs). Finally, PARP13 is thus far the only PARP for which catalytic activity could not be demonstrated either in vitro or in vivo. PARP14 is a cytosolic as well as nuclear monoPARP. It was originally identified as BAL2 (B Aggressive Lymphoma 2), a gene associated with inferior outcome of diffuse large B cell lymphoma (DLBCL), together with two other monoPARPs (PARP9 or BAL1 and PARP15 or BAL3) (Aguiar R C, et al. Blood. 2000 Dec. 9; 96(13):4328-4334 and Juszczynski P, et al. Mol Cell Biol. 2006 Jul. 1; 26(14):5348-5359). PARP14, PARP9 and PARP15 are also referred to as macro-PARPs due to the presence of macro-domains in their N-terminus. The genes for the three macroPARPs are located in the same genomic locus suggesting co-regulation. Indeed, the gene expression of PARP14 and PARP9 is highly correlated across normal tissues and cancer types. PARP14 is overexpressed in tumors compared to normal tissues, including established cancer cell lines in comparison to their normal counterparts. Literature examples of cancers with high PARP14 expression are DLBCL (Aguiar R C T, et al. J Biol Chem. 2005 Aug. 1; 280(40):33756-33765), multiple myeloma (MM) (Barbarulo A, et al. Oncogene. 2012 Oct. 8; 32(36):4231-4242) and hepatocellular carcinoma (HCC) (Iansante V, et al. Nat Commun. 2015 Aug. 10; 6:7882). In MM and HCC cell lines RNA interference (RNAi) mediated PARP14 knockdown inhibits cell proliferation and survival. Other studies show that the enzymatic activity of PARP14 is required for survival of prostate cancer cell lines in vitro (Bachmann S B, et al. Mol Cancer. 2014 May 27; 13:125).

PARP14 has been identified as a downstream regulator of IFN-γ and IL-4 signaling, influencing transcription downstream of STAT1 (in the case of IFN-γ) (Iwata H, et al. Nat Commun. 2016 Oct. 31; 7:12849) or STAT6 (in the case of IL-4) (Goenka S, et al. Proc Natl Acad Sci USA. 2006 Mar. 6; 103(11):4210-4215; Goenka S, et al. J Biol Chem. 2007 May 3; 282(26):18732-18739; and Mehrotra P, et al. J Biol Chem. 2010 Nov. 16; 286(3):1767-1776). Parp14−/− knockout (KO) mice have reduced marginal zone B cells, and the ability of IL-4 to confer B cell survival in vitro was reduced as well in the Parp14 KO setting (Cho S H, et al. Blood. 2009 Jan. 15; 113(11):2416-2425). This decreased survival signaling was linked mechanistically to decreased abilities of Parp14 KO B cells to sustain metabolic fitness and to increased Mcl-1 expression. Parp14 KO can extend survival in the Ep-Myc lymphoma model, suggesting a role of PARP14 in Myc-driven lymphomagenesis (Cho S H, et al. Proc Natl Acad Sci USA. 2011 Sep. 12; 108(38):15972-15977). Gene expression data point towards roles of PARP14 in human B cell lymphoma as well. The BAL proteins, including PARP14, are highly expressed in host response (HR) DLBCLs, a genomically defined B cell lymphoma subtype characterized with a brisk inflammatory infiltrate of T and dendritic cells and presence of an IFN-γ gene signature (Molecular profiling of diffuse large B-cell lymphoma identifies robust subtypes including one characterized by host inflammatory response. Monti S, et al. Blood. 2005; 105(5):1851). Indeed, PARP14 is believed to be an interferon stimulated gene with its mRNA increased by stimulation of various cell systems with all types of interferon (I, II and III; www.interferome.org).

Due to its role downstream of IL-4 and IFN-γ signaling pathways PARP14 has been implicated in T helper cell and macrophage differentiation. Genetic PARP14 inactivation in macrophages skews to a pro-inflammatory M1 phenotype associated with antitumor immunity while reducing a pro-tumor M2 phenotype. M1 gene expression, downstream of IFN-γ, was found to be increased while M2 gene expression, downstream of IL-4, was decreased with PARP14 knockout or knockdown in human and mouse macrophage models. Similarly, genetic PARP14 knockout has been shown to reduce a Th2 T helper cell phenotype in the setting of skin and airway inflammation, again pertaining to the regulatory role of PARP14 in IL-4 signal transduction (Mehrotra P, et al. J Allergy Clin Immunol. 2012 Jul. 25; 131(2):521 and Krishnamurthy P, et al. Immunology. 2017 Jul. 27; 152(3):451-461).

PARP14 was shown to regulate the transcription of STAT6 (activator of transcription 6) and promotes TH2 responses in T cells and B cells, which are known to promote allergic airway disease (asthmatic condition). Genetic depletion of PARP14 and its enzymatic activity in a model of allergic airway disease led to reduced lung inflammation and IgE levels, which are key readouts of the asthmatic process in this model. In addition, the enzymatic activity of PARP14 promoted a TH2 phenotype differentiation in a STAT6 dependent manner. (Mehrotra P, et al. J Allergy Clin Immunol. 2012 Jul. 25; 131(2):521) Therefore, inhibition of the PARP14 catalytic activity may be a potential novel therapy for allergic airway disease.

Most clinically used pharmaceutical agents are based upon small-molecule inhibition of protein function. However, alternative approaches that provide for protein degradation, rather than inhibition, also have the potential to provide clinical efficacy. Accordingly, targeted protein degradation through ubiquitination of protein targets has emerged as an effective strategy in drug discovery. Heterobifunctional small molecules, which simultaneously bind to target proteins and recruit an ubiquitin ligase (e.g., ubiquitin E3 ligase) have been shown to result in the target protein's ubiquitination and degradation (Bondeson, D. P., et al. Nat Chem Biol. 2015 11(8):611-617).

There is a need for the development of new drugs, such as small molecules that can bind to both PARP14 and ubiquitin E3 ligase to cause PARP14 degradation, which are useful in the treatment of various diseases, including cancer and inflammatory diseases.

### SUMMARY OF THE INVENTION

The present invention is directed to a compound of Formula (A1):

Q-L¹-E (A1)

or a pharmaceutically acceptable salt thereof, wherein constituent members are defined below.

The present invention is further directed to a pharmaceutical composition comprising a compound of Formula (A1), or a pharmaceutically acceptable salt thereof, and at least one pharmaceutically acceptable carrier.

The present invention is further directed to a method of degrading PARP14, comprising contacting a compound of Formula (A1), or a pharmaceutically acceptable salt thereof, with PARP14.

The present invention is further directed to a method of decreasing IL-10 in a cell comprising contacting a compound of Formula (A1), or a pharmaceutically acceptable salt thereof, with the cell.

The present invention is further directed to a method of treating a disease or disorder in a patient in need of treatment, where the disease or disorder is characterized by overexpression or increased activity of PARP14, comprising administering to the patient a therapeutically effective amount of a compound Formula (A1), or a pharmaceutically acceptable salt thereof.

The present invention is further directed to a method of treating cancer in a patient in need thereof comprising administering to said patient a therapeutically effective amount of a compound of Formula (A1), or a pharmaceutically acceptable salt thereof.

The present invention is further directed to a method of treating an inflammatory disease in a patient in need of treatment comprising administering to said patient a therapeutically effective amount of a compound of Formula (A1), or a pharmaceutically acceptable salt thereof.

The present invention also provides uses of the compounds described herein in the manufacture of a medicament for use in therapy. The present disclosure also provides the compounds described herein for use in therapy.

### BRIEF DESCRIPTION OF THE DRAWINGS

FIG. 1 shows the Western blot of the PARP14 degradation assay for the compound of Example 1.

FIG. 2 shows the Western blot of the PARP14 degradation assay for the compound of Example 2.

FIG. 3 shows the Western blot of the PARP14 degradation assay for the compound of Example 3.

FIG. 4 shows the Western blot of the PARP14 degradation assay for the compound of Example 4.

FIG. 5 shows the mRNA expression levels of PARP14 in various cancer types, compared to their matched normal tissue.

FIG. 6A shows the experimental layout of the procedure described in Example D, relating to the reduction of IL-10 production in cells.

FIG. 6B shows IL-10 levels in tissue culture supernatant, measured by ELISA, of cells treated as described in Example D.

### DETAILED DESCRIPTION

The present disclosure provides, inter alia, a compound of Formula (A1):

Q-L¹-E (A1)

or a pharmaceutically acceptable salt thereof, wherein:

Q is a small molecule PARP14 targeting moiety, which binds to PARP14;

L¹ is a linker, which is covalently linked to moiety Q and to moiety E; and

E is an E3 ubiquitin ligase binding moiety, which binds to the E3 ubiquitin ligase.

In some embodiments, provided herein is a compound of Formula (A1):

Q-L¹-E (A1)

or a pharmaceutically acceptable salt thereof, wherein:

Q is a moiety represented by Formula I:

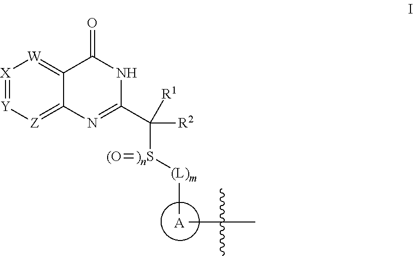

wherein:

W is CR^{W} or N;

X is CR^{X} or N;

Y is CR^{Y} or N;

Z is CR^{Z} or N;

wherein no more than two of W, X, Y, and Z are simultaneously N;

Ring A is monocyclic or polycyclic C₃₋₁₄ cycloalkyl or Ring A is monocyclic or polycyclic 4-18 membered heterocycloalkyl, wherein Ring A is optionally substituted by 1, 2, 3, or 4 R^{A}, and Ring A is attached to the -(L)ₘ- moiety of Formula I through a non-aromatic ring when Ring A is polycyclic;

L is —(CR⁵R⁶)ₜ—, —(CR⁵R⁶)ₚ—O—(CR⁵R⁶)_{q}—, —(CR⁵R⁶)ₚ—S—(CR⁵R⁶)_{q}—, —(CR⁵R⁶)ₚ—NR³—(CR⁵R⁶)_{q}—, —(CR⁵R⁶)ₚ—CO—(CR⁵R⁶)_{q}—, —(CR⁵R⁶)ᵣ—C(O)O—(CR⁵R⁶)ₛ—, —(CR⁵R⁶)ᵣ—CONR³—(CR⁵R⁶)ₛ—, —(CR⁵R⁶)ₚ—SO—(CR⁵R⁶)_{q}—, —(CR⁵R⁶)ₚ—SO₂—(CR⁵R⁶)_{q}—, —(CR⁵R⁶)ᵣ—SONR³—(CR⁵R⁶)ₛ—, or

—NR³CONR⁴—;

R¹ and R² are each, independently, selected from H and methyl;

R³ and R⁴ are each, independently, selected from H and C₁₋₄ alkyl;

R⁵ and R⁶ are each, independently, selected from H, halo, C₁₋₄ alkyl, C₁₋₄ alkoxy, C₁₋₄ haloalkyl, amino, C₁₋₄ alkylamino, and C₂-s dialkylamino;

each R^{A} is independently selected from halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{A} are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

R^{W}, R^{X}, R^{Y}, and R^{Z} are each, independently, selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{W}, R^{X}, R^{Y}, or R^{Z} are each optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR², C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};

wherein when W is CR^{W}, X is CR^{X}, Y is CR^{Y}, and Z is CR^{Z}, then at least one of R^{W}, R^{X}, R^{Y}, and R^{Z} is other than H;

each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

each Cy² is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};

each R^{a1}, R^{b1}, R^{c1}, R^{d1}, R^{a2}, R^{b2}, R^{c2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of Ra, R^{b1}, R^{c1}, R^{d1}, R^{a2}, R^{b2}, R^{c2}, or R^{d2} is optionally substituted with 1, 2, 3, 4, or 5 substituents independently selected from Cy³, Cy³-C₁₋₄ alkyl, halo, C₁₋₄ alkyl, C₁₋₄haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};

each Cy³ is C₆₋₁₀ aryl, C₃₋₇ cycloalkyl, 5-10 membered heteroaryl, or 4-10 membered heterocycloalkyl, each optionally substituted by 1, 2, 3, or 4 substituents independently selected from halo, C₁₋₄ alkyl, C₁₋₄haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};

R^{a3}, R^{b3}, R^{c3}, and R^{d3} are independently selected from H, C₁₋₆ alkyl, C₁₋₆ haloalkyl, C₂-6 alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;

or R^{c1} and R^{d1} together with the N atom to which they are attached form a 4-7 membered heterocycloalkyl group optionally substituted with 1, 2, or 3 substituents independently selected from halo, C₁₋₄ alkyl, C₁₋₄haloalkyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3}; or R^{c2} and R^{d2} together with the N atom to which they are attached form a 4-7 membered heterocycloalkyl group optionally substituted with 1, 2, or 3 substituents independently selected from halo, C₁₋₄ alkyl, C₁₋₄haloalkyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};

each R^{e1}, R^{e2}, and R^{e3} is independently selected from H, C₁₋₄ alkyl, and CN;

m is 0 or 1,

n is 0, 1, or 2;

p is 0, 1, or 2;

q is 0, 1, or 2, wherein p+q is 0, 1, or 2;

r is 0 or 1;

s is 0 or 1, where r+s is 0 or 1; and

t is 1, 2, or 3;

L¹ is a linker, which is covalently linked to moiety Q and to moiety E;

E is an E3 ubiquitin ligase binding moiety, which binds to the E3 ubiquitin ligase; and

wherein the wavy lines represent the point of attachment to group L¹;

wherein any aforementioned heteroaryl or heterocycloalkyl group comprises 1, 2, 3, or 4 ring-forming heteroatoms independently selected from O, N, and S;

wherein one or more ring-forming C or N atoms of any aforementioned heterocycloalkyl group is optionally substituted by an oxo (═O) group; and

wherein one or more ring-forming S atoms of any aforementioned heterocycloalkyl group is optionally substituted by one or two oxo (═O) groups.

In some embodiments, when W is CR^{W}, X is CR^{X}, Y is CR^{Y}, and Z is CR^{Z} and when m is 1, then R^{X} and R^{Y} are not both methoxy.

In some embodiments, Q is a moiety other than:

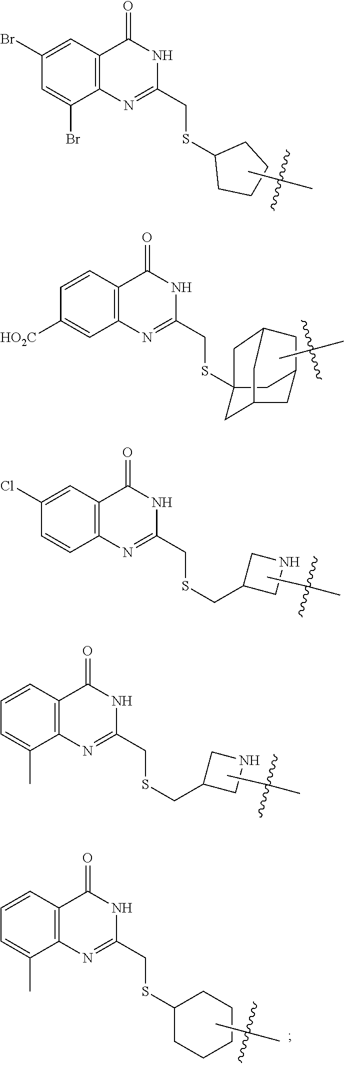

wherein the wavy lines represent the point of attachment to group L¹.

In some embodiments, W is CR^{W}; X is CR^{X}; Y is CR^{Y}; and Z is CR^{Z}.

In some embodiments, W is N; X is CR^{X}; Y is CR^{Y}; and Z is CR^{Z}.

In some embodiments, W is CR^{W}; X is N; Y is CR^{Y}; and Z is CR^{Z}.

In some embodiments, W is CR^{W}; X is CR^{X}; Y is N; and Z is CR^{Z}.

In some embodiments, W is CR^{W}; X is CR^{X}; Y is CR^{Y}; and Z is N.

In some embodiments, Ring A is monocyclic or polycyclic C₃₋₁₄ cycloalkyl optionally substituted by 1, 2, 3, or 4 R^{A}, wherein Ring A is attached to the -(L)ₘ- moiety of Formula I through a non-aromatic ring when Ring A is polycyclic.

In some embodiments, Ring A is monocyclic C₃₋₇ cycloalkyl optionally substituted by 1, 2, 3, or 4 RA.

In some embodiments, Ring A is cyclobutyl, cyclopentyl, cyclohexyl, or cycloheptyl optionally substituted by 1, 2, 3, or 4 R^{A}.

In some embodiments, Ring A is cyclobutyl, cyclopentyl, cyclohexyl, or cycloheptyl.

In some embodiments, Ring A is cyclohexyl or cycloheptyl optionally substituted by 1, 2, 3, or 4 RA.

In some embodiments, Ring A is cyclohexyl or cycloheptyl.

In some embodiments, Ring A is cyclohexyl optionally substituted by 1, 2, 3, or 4 R^{A}.

In some embodiments, Ring A is cyclohexyl.

In some embodiments, Ring A is monocyclic or polycyclic 4-18 membered heterocycloalkyl optionally substituted by 1, 2, 3, or 4 R^{A}, and wherein Ring A is attached to the -(L)ₘ- moiety of Formula I through a non-aromatic ring when Ring A is polycyclic.

In some embodiments, Ring A is monocyclic 4-7 membered heterocycloalkyl optionally substituted by 1, 2, 3, or 4 R^{A}.

In some embodiments, Ring A is monocyclic 4-7 membered heterocycloalkyl.

In some embodiments, Ring A is oxetanyl, tetrahydropyranyl, oxepanyl, azetidinyl, pyrrolidinyl, piperidinyl, or azepanyl, optionally substituted by 1, 2, 3, or 4 R^{A}.

In some embodiments, Ring A is oxetanyl, tetrahydropyranyl, oxepanyl, azetidinyl, pyrrolidinyl, piperidinyl, or azepanyl.

In some embodiments, Ring A is oxetanyl, tetrahydropyranyl, oxepanyl, azetidinyl, pyrrolidinyl, piperidinyl, azepanyl, or tetrahydrothiopyranyl optionally substituted by 1, 2, 3, or 4 R^{A}.

In some embodiments, Ring A is oxetanyl, tetrahydropyranyl, oxepanyl, azetidinyl, pyrrolidinyl, piperidinyl, azepanyl, or tetrahydrothiopyranyl.

In some embodiments, Ring A is piperidinyl optionally substituted by 1, 2, 3, or 4 R^{A}.

In some embodiments, Ring A is piperidinyl.

In some embodiments, Ring A is piperidin-4-yl optionally substituted by 1, 2, 3, or 4 RA.

In some embodiments, Ring A is piperidin-4-yl.

In some embodiments, Ring A is tetrahydropyranyl optionally substituted by 1, 2, 3, or 4 R^{A}.

In some embodiments, Ring A is tetrahydropyranyl.

In some embodiments, Ring A is tetrahydropyran-4-yl optionally substituted by 1, 2, 3, or 4 R^{A}.

In some embodiments, Ring A is tetrahydropyran-4-yl.

In some embodiments, L is —(CR⁵R⁶)ₜ—.

In some embodiments, L is —(CR⁵R⁶)ₜ— and t is 1.

In some embodiments, L is —(CR⁵R⁶)ₜ— and t is 2.

In some embodiments, L is —(CR⁵R⁶)ₜ— and t is 3.

In some embodiments, L is —CH₂—.

In some embodiments, m is 0.

In some embodiments, m is 1.

In some embodiments, n is 0.

In some embodiments, n is 1.

In some embodiments, n is 2.

In some embodiments, R¹ and R² are both H.

In some embodiments, one of R¹ and R² is H and the other is methyl.

In some embodiments, each R^{A} is independently selected from C₁₋₆ alkyl, OR^{a1}, C(O)R^{b1}, NR^{c1}R^{d1}, and S(O)₂R^{b1}; wherein said C₁₋₆ alkyl is optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NRC‘Rd’, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

In some embodiments, each R^{A} is independently selected from C₁₋₆ alkyl, halo, C₁₋₆ haloalkyl, OR^{a1}, C(O)R^{b1}, C(O)NR^{c1}R^{d1}, C(O)OR^{a1}, NR^{c1}R^{d1}, S(O)₂R^{b1}, 4-10 membered heterocycloalkyl, 5-10 membered heteroaryl, 4-10 membered heterocycloalkyl-C₁₋₄ alkyl and 5-10 membered heteroaryl-C₁₋₄ alkyl; wherein said C₁₋₆ alkyl, C₁₋₆ haloalkyl, 4-10 membered heterocycloalkyl, 5-10 membered heteroaryl, 4-10 membered heterocycloalkyl-C₁₋₄ alkyl and 5-10 membered heteroaryl-C₁₋₄ alkyl are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂-6 alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

In some embodiments, each R^{A} is independently selected from halo, C₁₋₆ haloalkyl, OR^{a1}, C(O)NR^{c1}R^{d1}, and C(O)OR^{a1}.

In some embodiments, R^{A} is OR^{a1}.

In some embodiments, each R^{A} is independently selected from halo, C₁₋₆ alkyl, C₁₋₆ haloalkyl, C₆₋₁₀ aryl-C₁₋₄ alkyl, 5-10 membered heteroaryl-C₁₋₄ alkyl, 4-10 membered heterocycloalkyl-C₁₋₄ alkyl, CN, OR^{a1}, NR^{c1}R^{d1}, C(O)NR^{c1}R^{d1}, NR^{c1}C(O)R^{b1}, C(O)R^{b1}, C(O)OR^{a1}, and S(O)₂R^{b1}, wherein said C₁₋₆ alkyl, C₁₋₆ haloalkyl, C₆₋₁₀ aryl-C₁₋₄ alkyl, 5-10 membered heteroaryl-C₁₋₄ alkyl, and 4-10 membered heterocycloalkyl-C₁₋₄ alkyl are each optionally substituted with 1, 2, 3, 4, or 5 substituents independently selected from halo, CN, OR^{a1}, NR^{c1}R^{d1}, C(O)R^{b1}, and NR^{c1}C(O)R^{b1}.

In some embodiments, each R^{W}, R^{X}, R^{Y}, and R^{Z} is independently selected from H, halo, C₁₋₆ alkyl, C₁₋₆ haloalkyl, 5-10 membered heteroaryl, 4-10 membered heterocycloalkyl, C₆₋₁₀ aryl-C₁₋₄ alkyl, CN, OR^{a2}, C(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, and NR^{c2}S(O)₂NR^{c2}R^{d2}; wherein said C₁₋₆ alkyl, C₁₋₆ haloalkyl, 5-10 membered heteroaryl, 4-10 membered heterocycloalkyl, and C₆₋₁₀ aryl-C₁₋₄ alkyl of R^{W}, R^{X}, R^{Y}, and R^{Z} are each optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}.

In some embodiments, each R^{W}, R^{X}, R^{Y}, and R^{Z} is independently selected from H, halo, C₁₋₆ alkyl, C₁₋₆ haloalkyl, 5-10 membered heteroaryl, 4-10 membered heterocycloalkyl, C₆₋₁₀ aryl-C₁₋₄ alkyl, CN, OR^{a2}, C(O)NR^{c2}R^{d2}, NR^{c2}R^{d2}, and NR^{c2}C(O)R^{b2}; wherein said C₁₋₆ alkyl, C₁₋₆ haloalkyl, 5-10 membered heteroaryl, 4-10 membered heterocycloalkyl, and C₆₋₁₀ aryl-C₁₋₄ alkyl of R^{W}, R^{X}, R^{Y}, and R^{Z} are each optionally substituted with 1, 2, 3, 4, or 5 substituents independently selected from Cy², Cy²-C₁₋₄ alkyl, halo, C₁₋₆ alkyl, C₂₋₆ alkenyl, C₂-6 alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2} In some embodiments, W is CR^{W} and R^{W} is other than H.

In some embodiments, W is CR^{W} and R^{W} is H.

In some embodiments, R^{W} is halo.

In some embodiments, R^{W} is F.

In some embodiments, R^{W} is selected from C₁₋₆ alkyl, C₁₋₆ haloalkyl, halo, and OR², wherein said C₁₋₆ alkyl and C₁₋₆ haloalkyl are each optionally substituted with OR^{a2}.

In some embodiments, R^{W} is selected from C₁₋₆ alkyl, C₁₋₆ haloalkyl, CN, halo, and OR^{a2}, wherein said C₁₋₆ alkyl and C₁₋₆ haloalkyl are each optionally substituted with OR^{a2}.

In some embodiments, R^{X} and R^{Z} are not both halogen.

In some embodiments, R^{Z} is H.

In some embodiments, when W is CR^{W}, X is CR^{X}, Y is CR^{Y}, and Z is CR^{Z} and when m is 1 or 2, then R^{X} and R^{Y} are not both C₁₋₆ alkoxy.

In some embodiments, when W is CR^{W}, X is CR^{X}, Y is CR^{Y}, and Z is CR^{Z} and when m is 1 or 2, then R^{X} and R^{Y} are not the same.

In some embodiments, X is CR^{X} and R^{X} is other than H.

In some embodiments, X is CR^{X} and R^{X} is H.

In some embodiments, R^{X} is selected from C₁₋₆ alkyl, halo, and OR^{a2}.

In some embodiments, Y is CR^{Y} and R^{Y} is other than H.

In some embodiments, Y is CR^{Y} and R^{Y} is H.

In some embodiments, Y is CR^{Y} and R^{Y} is independently selected from NR²R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, and NR^{c2}S(O)₂NR^{c2}R^{d2}.

In some embodiments, Y is CR^{Y} and R^{Y} is independently selected from C₁₋₆ alkyl, OR^{a2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, and NR^{c2}S(O)₂NR^{c2}R^{d2}.

In some embodiments, Y is CR^{Y} and R^{Y} is independently selected from NR²R^{d2} and NR^{c2}C(O)R^{b2}.

In some embodiments, R^{Y} is independently selected from C₁₋₆ alkyl, C₃₋₇ cycloalkyl-C₁₋₄ alkyl, 5-10 membered heteroaryl, 4-10 membered heterocycloalkyl, halo, CN, OR², SR^{a2}, C(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, and NR^{c2}S(O)₂NR^{c2}R^{d2}, wherein said C₁₋₆ alkyl, C₃₋₇ cycloalkyl-C₁₋₄ alkyl, 5-10 membered heteroaryl, and 4-10 membered heterocycloalkyl of R are each optionally substituted with 1, 2, 3, 4, or 5 substituents independently selected from halo, C₁₋₆ alkyl, C₁₋₆ haloalkyl, CN, NO₂, OR^{a2}, NR^{c2}R^{d2}, and S(O)₂R^{b2}.

In some embodiments, Y is CR^{Y} and R^{Y} is independently selected from C₁₋₆ alkyl and OR^{a2}.

In some embodiments, Y is CR^{Y} and R^{Y} is OR^{a2}.

In some embodiments, Z is CR^{Z} and R^{Z} is other than H.

In some embodiments, Z is CR^{Z} and R^{Z} is H.

In some embodiments, Z is CR^{Z} and R^{Z} is C₁₋₆ alkyl.

In some embodiments, Z is CR^{Z} and R^{Z} is C₁₋₆ alkyl, halo, or CN.

In some embodiments, each R^{a1}, R^{b1}, R^{c1}, R^{d1}, R^{a2}, R^{b2}, R^{c2}, and R^{d2} is independently selected from H, C₁₋₆ alkyl, and C₁₋₆ haloalkyl, wherein the C₁₋₆ alkyl is optionally substituted with 1, 2, 3, 4, or 5 substituents independently selected from Cy³, Cy³-C₁₋₄ alkyl, halo, C₁₋₄ alkyl, C₁₋₄ haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3}.

In some embodiments, each R^{a1}, R^{b1}, R^{c1}, R^{d1}, R^{a2}, R^{b2}, R^{c2}, and R^{d2} is independently selected from H, C₁₋₆ alkyl, and C₁₋₆ haloalkyl.

In some embodiments, each R^{a1}, R^{b1}, R^{c1}, R^{d1}, R^{a2}, R^{b2}, R^{c2}, and R^{d2} is independently selected from H, C₁₋₆ alkyl, C₁₋₆ haloalkyl, C₆₋₁₀ aryl, C₃₋₇ cycloalkyl, 4-10 membered heterocycloalkyl, C₆₋₁₀ aryl-C₁₋₄ alkyl, C₃₋₇ cycloalkyl-C₁₋₄ alkyl, and 4-10 membered heterocycloalkyl-C₁₋₄ alkyl, wherein said C₁₋₆ alkyl, C₁₋₆ haloalkyl, C₆₋₁₀ aryl, C₃₋₇ cycloalkyl, 4-10 membered heterocycloalkyl, C₆₋₁₀ aryl-C₁₋₄ alkyl, C₃₋₇ cycloalkyl-C₁₋₄ alkyl, and 4-10 membered heterocycloalkyl-C₁₋₄ alkyl are each optionally substituted with 1, 2, 3, 4, or 5 substituents independently selected from C₁₋₄ alkyl, C₁₋₄ haloalkyl, halo, CN, OR^{a3}, C(O)R^{b3}, C(O)OR^{a3} and S(O)₂R^{b3}.

In some embodiments, R^{a2} is selected from H, C₁₋₆ alkyl, C₁₋₆ haloalkyl, C₆₋₁₀ aryl, C₃₋₇ cycloalkyl, 4-10 membered heterocycloalkyl, C₆₋₁₀ aryl-C₁₋₄ alkyl, C₃₋₇ cycloalkyl-C₁₋₄ alkyl, and 4-10 membered heterocycloalkyl-C₁₋₄ alkyl, wherein said C₁₋₆ alkyl, C₁₋₆ haloalkyl, C₆₋₁₀ aryl, C₃₋₇ cycloalkyl, 4-10 membered heterocycloalkyl, C₆₋₁₀ aryl-C₁₋₄ alkyl, C₃₋₇ cycloalkyl-C₁₋₄ alkyl, and 4-10 membered heterocycloalkyl-C₁₋₄ alkyl are each optionally substituted with 1, 2, 3, 4, or 5 substituents independently selected from C₁₋₄ alkyl, C₁₋₄ haloalkyl, halo, CN, OR^{a3}, C(O)R^{b3}, C(O)OR^{a3} and S(O)₂R^{b3}.

In some embodiments, R^{c2} and R^{d2} are each independently selected from H, C₁₋₆ alkyl, C₁₋₆ haloalkyl, C₆₋₁₀ aryl, C₃₋₇ cycloalkyl, 4-10 membered heterocycloalkyl, C₆₋₁₀ aryl-C₁₋₄ alkyl, C₃₋₇ cycloalkyl-C₁₋₄ alkyl, and 4-10 membered heterocycloalkyl-C₁₋₄ alkyl, wherein said C₁₋₆ alkyl, C₁₋₆ haloalkyl, C₆₋₁₀ aryl, C₃₋₇ cycloalkyl, 4-10 membered heterocycloalkyl, C₆₋₁₀ aryl-C₁₋₄ alkyl, C₃₋₇ cycloalkyl-C₁₋₄ alkyl, and 4-10 membered heterocycloalkyl-C₁₋₄ alkyl are each optionally substituted with 1, 2, 3, 4, or 5 substituents independently selected from C₁₋₄ alkyl, C₁₋₄haloalkyl, halo, CN, OR^{a3}, C(O)R^{b3}, C(O)OR^{a3} and S(O)₂R^{b3}.

In some embodiments, Cy³ is 4-10 membered heterocycloalkyl optionally substituted with 1, 2, 3, 4, or 5 substituents independently selected from C₁₋₄ alkyl, C₁₋₄ haloalkyl, halo, CN, OR^{a3}, C(O)R^{b3}, C(O)OR^{a3} and S(O)₂R^{b3}.

In some embodiments, Cy³ is 4-10 membered heterocycloalkyl optionally substituted with 1, 2, 3, 4, or 5 substituents independently selected from C(O)R^{b3}.

In some embodiments, Cy³ is piperidinyl optionally substituted with 1, 2, 3, 4, or 5 substituents independently selected from halo and C(O)CH₃.

In some embodiments, Q is a moiety represented by Formula II:

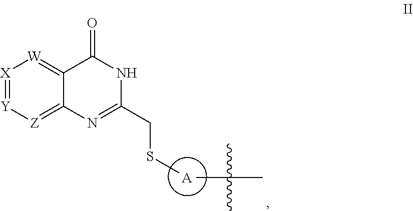

wherein the wavy line represents the point of attachment to group L¹.

In some embodiments, Q is a moiety represented by Formula IIIA, IIIB, IIIC, HID, or IIIE:

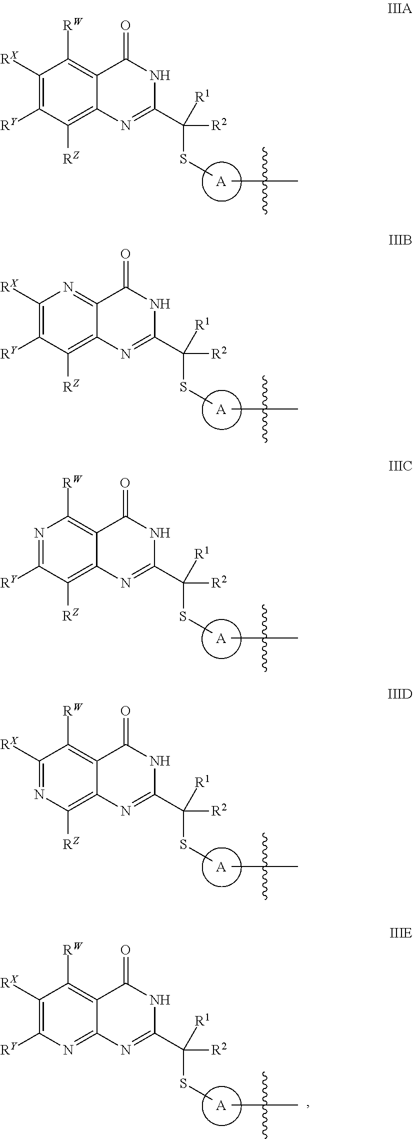

wherein the wavy lines represent the point of attachment to group L¹.

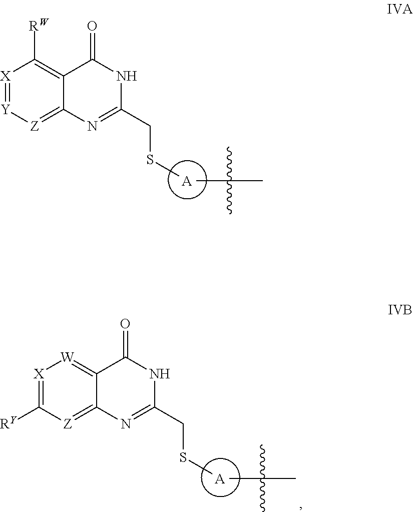

wherein the wavy lines represent the point of attachment to group L¹.

In some embodiments, Q is a radical of a compound selected from:
4-oxo-2-(((tetrahydro-2H-pyran-4-yl)thio)methyl)-3,4-dihydroquinazoline-7-carbonitrile;
8-methyl-2-(((tetrahydro-2H-pyran-4-yl)thio)methyl)quinazolin-4(3H)-one;
6-methyl-2-(((tetrahydro-2H-pyran-4-yl)thio)methyl)quinazolin-4(3H)-one;
6-methoxy-2-(((tetrahydro-2H-pyran-4-yl)thio)methyl)quinazolin-4(3H)-one;
8-chloro-2-(((tetrahydro-2H-pyran-4-yl)thio)methyl)quinazolin-4(3H)-one;
8-methoxy-2-(((tetrahydro-2H-pyran-4-yl)thio)methyl)quinazolin-4(3H)-one;
8-methyl-2-(1-((tetrahydro-2H-pyran-4-yl)thio)ethyl)quinazolin-4(3H)-one;
5-fluoro-8-methyl-2-(((tetrahydro-2H-pyran-4-yl)thio)methyl)quinazolin-4(3H)-one;
5-methyl-2-(((tetrahydro-2H-pyran-4-yl)thio)methyl)quinazolin-4(3H)-one;
8-benzyl-2-(((tetrahydro-2H-pyran-4-yl)thio)methyl)quinazolin-4(3H)-one;
7-benzyl-2-(((tetrahydro-2H-pyran-4-yl)thio)methyl)quinazolin-4(3H)-one;
8-Methyl-2-((((tetrahydro-2H-pyran-4-yl)methyl)thio)methyl)quinazolin-4(3H)-one;
8-Methyl-2-((piperidin-4-ylthio)methyl)quinazolin-4(3H)-one trifluoroacetate;
8-Methyl-2-(((1-methylpiperidin-4-yl)thio)methyl)quinazolin-4(3H)-one;
8-Methyl-2-((pyrrolidin-3-ylthio)methyl)quinazolin-4(3H)-one;
8-Methyl-2-(((1-methylpyrrolidin-3-yl)thio)methyl)quinazolin-4(3H)-one;
2-(((1-Acetylpiperidin-4-yl)thio)methyl)-8-methylquinazolin-4(3H)-one;
8-Methyl-2-(((1-(pyridin-2-ylmethyl)piperidin-4-yl)thio)methyl)quinazolin-4(3H)-one;
8-Methyl-2-(((tetrahydro-2H-pyran-4-yl)sulfonyl)methyl)quinazolin-4(3H)-one;
2-((Azepan-4-ylthio)methyl)-8-methylquinazolin-4(3H)-one;
2-(((4-(Dimethylamino)cyclohexyl)thio)methyl)-8-methylquinazolin-4(3H)-one;
2-(((4-Hydroxycyclohexyl)thio)methyl)-8-methylquinazolin-4(3H)-one;
2-((((trans)-4-Hydroxycyclohexyl)thio)methyl)-8-methylquinazolin-4(3H)-one;
2-((((cis)-4-Hydroxycyclohexyl)thio)methyl)-8-methylquinazolin-4(3H)-one;
2-((Azetidin-3-ylthio)methyl)-8-methylquinazolin-4(3H)-one;
2-((((trans)-4-Methoxycyclohexyl)thio)methyl)-8-methylquinazolin-4(3H)-one;
2-((((cis)-4-Methoxycyclohexyl)thio)methyl)-8-methylquinazolin-4(3H)-one;
4-Oxo-2-(((tetrahydro-2H-pyran-4-yl)thio)methyl)-3,4-dihydroquinazoline-8-carbonitrile;
7-Phenoxy-2-(((tetrahydro-2H-pyran-4-yl)thio)methyl)quinazolin-4(3H)-one;
7-Fluoro-2-(((tetrahydro-2H-pyran-4-yl)thio)methyl)quinazolin-4(3H)-one;
7-Methoxy-2-(((tetrahydro-2H-pyran-4-yl)thio)methyl)quinazolin-4(3H)-one;
8-Methyl-2-(((1-methylpiperidin-3-yl)thio)methyl)quinazolin-4(3H)-one;
7-Fluoro-8-methyl-2-(((tetrahydro-2H-pyran-4-yl)thio)methyl)quinazolin-4(3H)-one;
5-Chloro-8-methyl-2-(((tetrahydro-2H-pyran-4-yl)thio)methyl)quinazolin-4(3H)-one;
8-Methyl-2-(((tetrahydro-2H-pyran-4-yl)thio)methyl)-5-(trifluoromethyl)quinazolin-4(3H)-one;
2-(((Tetrahydro-2H-pyran-4-yl)thio)methyl)pyrido[3,2-d]pyrimidin-4(3H)-one;
2-(((Tetrahydro-2H-pyran-4-yl)thio)methyl)pyrido[3,4-d]pyrimidin-4(3H)-one;
2-((((trans)-3-(Benzyloxy)cyclobutyl)thio)methyl)-8-methylquinazolin-4(3H)-one;
8-Methyl-2-((oxetan-3-ylthio)methyl)quinazolin-4(3H)-one;
2-(((Tetrahydro-2H-pyran-4-yl)thio)methyl)pyrido[4,3-d]pyrimidin-4(3H)-one;
8-Methyl-2-(((tetrahydro-2H-pyran-4-yl)thio)methyl)pyrido[3,2-d]pyrimidin-4(3H)-one;
8-Methyl-2-(((tetrahydro-2H-pyran-4-yl)thio)methyl)pyrido[3,4-d]pyrimidin-4(3H)-one;
2-(((Tetrahydro-2H-pyran-4-yl)thio)methyl)pyrido[2,3-d]pyrimidin-4(3H)-one;
6-Chloro-8-methyl-2-(((tetrahydro-2H-pyran-4-yl)thio)methyl)quinazolin-4(3H)-one;
7,8-Difluoro-2-(((tetrahydro-2H-pyran-4-yl)thio)methyl)quinazolin-4(3H)-one;
7-Fluoro-2-((((trans)-4-hydroxycyclohexyl)thio)methyl)quinazolin-4(3H)-one;
2-(((trans-3-Hydroxycyclobutyl)thio)methyl)-8-methylquinazolin-4(3H)-one;
8-Methyl-2-((piperidin-3-ylthio)methyl)quinazolin-4(3H)-one;
2-(((trans-4-Aminocyclohexyl)thio)methyl)-8-methylquinazolin-4(3H)-one;
2-(((cis-4-Aminocyclohexyl)thio)methyl)-8-methylquinazolin-4(3H)-one;
5-Fluoro-8-methyl-2-((piperidin-4-ylthio)methyl)quinazolin-4(3H)-one;
2-(((trans-3-Aminocyclobutyl)thio)methyl)-8-methylquinazolin-4(3H)-one;
2-(((4-Aminocycloheptyl)thio)methyl)-8-methylquinazolin-4(3H)-one;
2-(((trans-4-Aminocycloheptyl)thio)methyl)-8-methylquinazolin-4(3H)-one;
2-(((cis-4-Aminocycloheptyl)thio)methyl)-8-methylquinazolin-4(3H)-one;
5-Fluoro-2-(((4-hydroxycyclohexyl)thio)methyl)-8-methylquinazolin-4(3H)-one;
5-Fluoro-2-(((trans-4-hydroxycyclohexyl)thio)methyl)-8-methylquinazolin-4(3H)-one;
5-Fluoro-2-(((cis-4-hydroxycyclohexyl)thio)methyl)-8-methylquinazolin-4(3H)-one;
2-(((4-Hydroxycyclohexyl)thio)methyl)-8-methyl-5-(trifluoromethyl)quinazolin-4(3H)-one;
2-(((trans-4-Hydroxycyclohexyl)thio)methyl)-8-methyl-5-(trifluoromethyl) quinazolin-4(3H)-one;
2-(((cis-4-Hydroxycyclohexyl)thio)methyl)-8-methyl-5-(trifluoromethyl) quinazolin-4(3H)-one;
2-(((trans-4-(Hydroxymethyl)cyclohexyl)thio)methyl)-8-methylquinazolin-4(3H)-one;
2-(((cis-4-(Hydroxymethyl)cyclohexyl)thio)methyl)-8-methylquinazolin-4(3H)-one;
2-(((4-(Aminomethyl)cyclohexyl)thio)methyl)-8-methylquinazolin-4(3H)-one;
2-(((cis-4-(Aminomethyl)cyclohexyl)thio)methyl)-8-methylquinazolin-4(3H)-one;
2-(((trans-4-(Aminomethyl)cyclohexyl)thio)methyl)-8-methylquinazolin-4(3H)-one;
2-(((4-((Dimethylamino)methyl)cyclohexyl)thio)methyl)-8-methylquinazolin-4(3H)-one;
2-(((cis-4-((Dimethylamino)methyl)cyclohexyl)thio)methyl)-8-methyl quinazolin-4(3H)-one;
2-(((trans-4-((Dimethylamino)methyl)cyclohexyl)thio)methyl)-8-methyl quinazolin-4(3H)-one;
2-(((trans-3-(Hydroxymethyl)cyclohexyl)thio)methyl)-8-methylquinazolin-4(3H)-one;
2-(((cis-3-(Hydroxymethyl)cyclohexyl)thio)methyl)-8-methylquinazolin-4(3H)-one:
2-((((cis)-3-((Dimethylamino)methyl)cyclohexyl)thio)methyl)-8-methyl quinazolin-4(3H)-one;
8-Methyl-2-(((trans-4-((methylamino)methyl)cyclohexyl)thio)methyl) quinazolin-4(3H)-one;
7-Amino-2-(((tetrahydro-2H-pyran-4-yl)thio)methyl)quinazolin-4(3H)-one;
N-(4-Oxo-2-(((tetrahydro-2H-pyran-4-yl)thio)methyl)-3,4-dihydroquinazolin-7-yl)acetamide;
N-(4-Oxo-2-(((tetrahydro-2H-pyran-4-yl)thio)methyl)-3,4-dihydroquinazolin-7-yl)benzamide;
N-Methyl-4-oxo-2-(((tetrahydro-2H-pyran-4-yl)thio)methyl)-3,4-dihydro quinazoline-7-carboxamide;
4-Oxo-N-phenyl-2-(((tetrahydro-2H-pyran-4-yl)thio)methyl)-3,4-dihydro quinazoline-7-carboxamide;
7-(Phenylamino)-2-(((tetrahydro-2H-pyran-4-yl)thio)methyl)quinazolin-4(3H)-one;
7-(Pyridin-3-ylamino)-2-(((tetrahydro-2H-pyran-4-yl)thio)methyl)quinazolin-4(3H)-one;
7-(Pyridin-2-ylamino)-2-(((tetrahydro-2H-pyran-4-yl)thio)methyl)quinazolin-4(3H)-one;
7-((4-Methoxyphenyl)amino)-2-(((tetrahydro-2H-pyran-4-yl)thio)methyl)quinazolin-4(3H)-one;
7-((3-Methoxyphenyl)amino)-2-(((tetrahydro-2H-pyran-4-yl)thio)methyl)quinazolin-4(3H)-one;
7-((2-Methoxyphenyl)amino)-2-(((tetrahydro-2H-pyran-4-yl)thio)methyl)quinazolin-4(3H)-one;
7-(Pyrazin-2-ylamino)-2-(((tetrahydro-2H-pyran-4-yl)thio)methyl)quinazolin-4(3H)-one;
7-(Pyridin-4-ylamino)-2-(((tetrahydro-2H-pyran-4-yl)thio)methyl)quinazolin-4(3H)-one;
7-(Pyrimidin-5-ylamino)-2-(((tetrahydro-2H-pyran-4-yl)thio)methyl)quinazolin-4(3H)-one;
7-((1-Methyl-1H-imidazol-2-yl)amino)-2-(((tetrahydro-2H-pyran-4-yl)thio)methyl)quinazolin-4(3H)-one;
2-(((Tetrahydro-2H-pyran-4-yl)thio)methyl)-7-(thiazol-2-ylamino)quinazolin-4(3H)-one;
7-((2-Methylpyridin-3-yl)amino)-2-(((tetrahydro-2H-pyran-4-yl)thio)methyl)quinazolin-4(3H)-one;
7-((4-Methylpyridin-3-yl)amino)-2-(((tetrahydro-2H-pyran-4-yl)thio)methyl)quinazolin-4(3H)-one;
7-((5-Methylpyridin-3-yl)amino)-2-(((tetrahydro-2H-pyran-4-yl)thio)methyl)quinazolin-4(3H)-one;
7-(4-Amino-1H-pyrazol-1-yl)-2-(((tetrahydro-2H-pyran-4-yl)thio)methyl) quinazolin-4(3H)-one;
7-(Isoxazol-3-ylamino)-2-(((tetrahydro-2H-pyran-4-yl)thio)methyl)quinazolin-4(3H)-one;
8-Methyl-7-(phenylamino)-2-(((tetrahydro-2H-pyran-4-yl)thio)methyl) quinazolin-4(3H)-one;
7-(Benzyloxy)-2-(((tetrahydro-2H-pyran-4-yl)thio)methyl)quinazolin-4(3H)-one;
2-(((4-Hydroxycyclohexyl)thio)methyl)-7-(phenylamino)quinazolin-4(3H)-one;
2-(((trans-4-Hydroxycyclohexyl)thio)methyl)-7-(phenylamino)quinazolin-4(3H)-one;
2-(((cis-4-Hydroxycyclohexyl)thio)methyl)-7-(phenylamino)quinazolin-4(3H)-one;
2-(((cis-4-Hydroxycyclohexyl)thio)methyl)-7-(pyridin-3-ylamino)quinazolin-4(3H)-one;
2-(((trans-4-Hydroxycyclohexyl)thio)methyl)-7-(pyridin-3-ylamino)quinazolin-4(3H)-one;
7-(Cyclopentylamino)-2-(((trans-4-hydroxycyclohexyl)thio)methyl) quinazolin-4(3H)-one;
7-(Cyclopentylamino)-2-(((cis-4-hydroxycyclohexyl)thio)methyl) quinazolin-4(3H)-one;
2-(((trans-4-(Hydroxymethyl)cyclohexyl)thio)methyl)-7-(phenylamino) quinazolin-4(3H)-one;
2-(((cis-4-(Hydroxymethyl)cyclohexyl)thio)methyl)-7-(phenylamino) quinazolin-4(3H)-one;
2-(((cis-4-(Hydroxymethyl)cyclohexyl)thio)methyl)-7-(pyridin-3-ylamino) quinazolin-4(3H)-one;
2-(((trans-4-(Hydroxymethyl)cyclohexyl)thio)methyl)-7-(pyridin-3-ylamino) quinazolin-4(3H)-one;
7-(Cyclohexylamino)-2-(((tetrahydro-2H-pyran-4-yl)thio)methyl)quinazolin-4(3H)-one;
7-(Dimethylamino)-2-(((tetrahydro-2H-pyran-4-yl)thio)methyl)quinazolin-4(3H)-one;
7-(Methylamino)-2-(((tetrahydro-2H-pyran-4-yl)thio)methyl)quinazolin-4(3H)-one;
7-Morpholino-2-(((tetrahydro-2H-pyran-4-yl)thio)methyl)quinazolin-4(3H)-one;
7-(4-Methylpiperazin-1-yl)-2-(((tetrahydro-2H-pyran-4-yl)thio)methyl)quinazolin-4(3H)-one;
7-((1-Methylpiperidin-4-yl)amino)-2-(((tetrahydro-2H-pyran-4-yl)thio)methyl)quinazolin-4(3H)-one;
7-((Tetrahydro-2H-pyran-4-yl)amino)-2-(((tetrahydro-2H-pyran-4-yl)thio)methyl)quinazolin-4(3H)-one;
7-(Cyclopentylamino)-2-(((tetrahydro-2H-pyran-4-yl)thio)methyl)quinazolin-4(3H)-one;
7-(Isopropylamino)-2-(((tetrahydro-2H-pyran-4-yl)thio)methyl)quinazolin-4(3H)-one;
7-((Pyridin-4-ylmethyl)amino)-2-(((tetrahydro-2H-pyran-4-yl)thio)methyl)quinazolin-4(3H)-one;
7-((Pyridin-2-ylmethyl)amino)-2-(((tetrahydro-2H-pyran-4-yl)thio)methyl)quinazolin-4(3H)-one;
7-(Benzylamino)-2-(((tetrahydro-2H-pyran-4-yl)thio)methyl)quinazolin-4(3H)-one;
7-((1-Phenylethyl)amino)-2-(((tetrahydro-2H-pyran-4-yl)thio)methyl)quinazolin-4(3H)-one;
2-(((Tetrahydro-2H-pyran-4-yl)thio)methyl)-7-((tetrahydrofuran-3-yl)amino)quinazolin-4(3H)-one;
7-(Cyclobutylamino)-2-(((tetrahydro-2H-pyran-4-yl)thio)methyl)quinazolin-4(3H)-one;
7-((Pyridin-3-ylmethyl)amino)-2-(((tetrahydro-2H-pyran-4-yl)thio)methyl)quinazolin-4(3H)-one;
7-(Cyclopropylamino)-2-(((tetrahydro-2H-pyran-4-yl)thio)methyl)quinazolin-4(3H)-one;
7-(Cyclohexyl(methyl)amino)-2-(((tetrahydro-2H-pyran-4-yl)thio)methyl)quinazolin-4(3H)-one;
7-[(1-Benzyl-3-piperidyl)amino]-2-(tetrahydropyran-4-ylsulfanylmethyl)-3H-quinazolin-4-one;
7-(3-Piperidylamino)-2-(tetrahydropyran-4-ylsulfanylmethyl)-3H-quinazolin-4-one;
7-((1-Benzylpiperidin-4-yl)amino)-2-(((tetrahydro-2H-pyran-4-yl)thio)methyl) quinazolin-4(3H)-one;
7-(Piperidin-4-ylamino)-2-(((tetrahydro-2H-pyran-4-yl)thio)methyl)quinazolin-4(3H)-one;
7-(Pyrrolidin-3-ylamino)-2-(((tetrahydro-2H-pyran-4-yl)thio)methyl)quinazolin-4(3H)-one;
7-((1-Acetylpiperidin-4-yl)amino)-2-(((tetrahydro-2H-pyran-4-yl)thio)methyl) quinazolin-4(3H)-one;
7-((1-Acetylpiperidin-3-yl)amino)-2-(((tetrahydro-2H-pyran-4-yl)thio)
methyl)quinazolin-4(3H)-one;
7-((1-Methylpiperidin-3-yl)amino)-2-(((tetrahydro-2H-pyran-4-yl)thio)
methyl)quinazolin-4(3H)-one
7-((1-Acetylpyrrolidin-3-yl)amino)-2-(((tetrahydro-2H-pyran-4-yl)thio)
methyl)quinazolin-4(3H)-one;
8-Methyl-7-phenoxy-2-(((tetrahydro-2H-pyran-4-yl)thio)methyl)quinazolin-4(3H)-one;
7-(Cyclohexylamino)-2-(((trans-4-(hydroxymethyl)cyclohexyl)thio)methyl)quinazolin-4(3H)-one;
7-(Cyclohexylamino)-2-(((cis-4-(hydroxymethyl)cyclohexyl)thio)methyl)quinazolin-4(3H)-one;
8-Methyl-2-(((1-((1-methyl-1H-imidazol-2-yl)methyl)piperidin-4-yl)thio)
methyl)quinazolin-4(3H)-one;
N-(4-((4-(((8-Methyl-4-oxo-3,4-dihydroquinazolin-2-yl)methyl)thio)piperidin-1-yl)methyl)phenyl)acetamide;
2-(((1-(4-(Dimethylamino)benzyl)piperidin-4-yl)thio)methyl)-8-methylquinazolin-4(3H)-one;
4-((4-(((8-Methyl-4-oxo-3,4-dihydroquinazolin-2-yl)methyl)thio)piperidin-1-yl)methyl)benzonitrile;
2-(((1-((1H-Pyrazol-3-yl)methyl)piperidin-4-yl)thio)methyl)-8-methylquinazolin-4(3H)-one;
8-Methyl-2-(((1-((1-methyl-1H-indazol-3-yl)methyl)piperidin-4-yl)thio)methyl)-quinazolin-4(3H)-one;
2-(((1-((1,3-Dimethyl-1H-pyrazol-4-yl)methyl)piperidin-4-yl)thio)methyl)-8-methylquinazolin-4(3H)-one;
8-Methyl-2-(((1-((6-methylpyridin-2-yl)methyl)piperidin-4-yl)thio)methyl)quinazolin-4(3H)-one;
8-Methyl-2-(((1-((3-methylpyridin-2-yl)methyl)piperidin-4-yl)thio)methyl)quinazolin-4(3H)-one;
8-Methyl-2-(((1-phenethylpiperidin-4-yl)thio)methyl)quinazolin-4(3H)-one;
8-Methyl-2-(((1-((1-methyl-1H-indazol-6-yl)methyl)piperidin-4-yl)thio)methyl)quinazolin-4(3H)-one;
8-Methyl-2-(((1-((3-methyl-1H-pyrazol-4-yl)methyl)piperidin-4-yl)thio)methyl)quinazolin-4(3H)-one;
N-(3-((4-(((8-Methyl-4-oxo-3,4-dihydroquinazolin-2-yl)methyl)thio)piperidin-1-yl)methyl)phenyl)acetamide;
2-(((1-((1H-Pyrrolo[3,2-c]pyridin-3-yl)methyl)piperidin-4-yl)thio)methyl)-8-methylquinazolin-4(3H)-one;
2-(((1-(Imidazo[1,2-a]pyridin-3-ylmethyl)piperidin-4-yl)thio)methyl)-8-methylquinazolin-4(3H)-one;
2-(((1-((1-Benzyl-1H-imidazol-5-yl)methyl)piperidin-4-yl)thio)methyl)-8-methylquinazolin-4(3H)-one;
2-(((1-((1-Benzyl-1H-pyrazol-4-yl)methyl)piperidin-4-yl)thio)methyl)-8-methylquinazolin-4(3H)-one;
2-(2-((4-(((8-Methyl-4-oxo-3,4-dihydroquinazolin-2-yl)methyl)thio)piperidin-1-yl)methyl)phenoxy)acetonitrile;
8-Methyl-2-(((1-((2-oxoindolin-6-yl)methyl)piperidin-4-yl)thio)methyl)quinazolin-4(3H)-one;
2-(((1-((5-Methoxypyridin-2-yl)methyl)piperidin-4-yl)thio)methyl)-8-methylquinazolin-4(3H)-one;
8-Methyl-2-(((1-((4-methyl-3,4-dihydro-2H-benzo[b][1,4]oxazin-7-yl)methyl)piperidin-4-yl)thio)methyl)quinazolin-4(3H)-one;
(S)-2-(((1-(2,3-Dihydroxypropyl)piperidin-4-yl)thio)methyl)-8-methylquinazolin-4(3H)-one;
(R)-2-(((1-(2,3-Dihydroxypropyl)piperidin-4-yl)thio)methyl)-8-methylquinazolin-4(3H)-one;
(S)-8-Methyl-2-(((1-(pyrrolidin-2-ylmethyl)piperidin-4-yl)thio)methyl)quinazolin-4(3H)-one;
2-(((1-(2-Hydroxyethyl)piperidin-4-yl)thio)methyl)-8-methylquinazolin-4(3H)-one;
2-(((1-(2-Aminoethyl)piperidin-4-yl)thio)methyl)-8-methylquinazolin-4(3H)-one;
N-(2-(4-(((8-Methyl-4-oxo-3,4-dihydroquinazolin-2-yl)methyl)thio)piperidin-1-yl)ethyl)picolinamide;
2-(((1-(3-Aminopropyl)piperidin-4-yl)thio)methyl)-8-methylquinazolin-4(3H)-one;
2-(((1-Glycylpiperidin-4-yl)thio)methyl)-8-methylquinazolin-4(3H)-one;
2-(((1-(3-Aminopropanoyl)piperidin-4-yl)thio)methyl)-8-methylquinazolin-4(3H)-one;
2-(((1-(3-(Dimethylamino)propanoyl)piperidin-4-yl)thio)methyl)-8-methylquinazolin-4(3H)-one;
(R)-1-(4-Amino-5-(4-(((8-methyl-4-oxo-3,4-dihydroquinazolin-2-yl)
methyl)thio)piperidin-1-yl)-5-oxopentyl)guanidine;
(S)-1-(4-Amino-5-(4-(((8-methyl-4-oxo-3,4-dihydroquinazolin-2-yl)methyl) thio)piperidin-1-yl)-5-oxopentyl)guanidine;
2-(((1-(L-Lysyl)piperidin-4-yl)thio)methyl)-8-methylquinazolin-4(3H)-one;
2-(((1-(D-Lysyl)piperidin-4-yl)thio)methyl)-8-methylquinazolin-4(3H)-one;
8-Methyl-2-(((1-(3-(pyridin-2-yl)propanoyl)piperidin-4-yl)thio)methyl)quinazolin-4(3H)-one;
8-Methyl-2-(((1-(methylsulfonyl)piperidin-4-yl)thio)methyl)quinazolin-4(3H)-one;
8-Methyl-2-(((1-(pyridin-2-ylsulfonyl)piperidin-4-yl)thio)methyl)quinazolin-4(3H)-one;
7-(Cyclopentylamino)-5-fluoro-2-((piperidin-4-ylthio)methyl)quinazolin-4(3H)-one;
and
7-(Cyclobutylamino)-5-fluoro-2-((piperidin-4-ylthio)methyl)quinazolin-4(3H)-one;
N-(((trans)-4-(((8-methyl-4-oxo-3,4-dihydroquinazolin-2-yl)methyl)thio)cyclohexyl)methyl)acetamide;
7-(cyclopentylamino)-2-((piperidin-4-ylthio)methyl)quinazolin-4(3H)-one;
7-(cyclopentylamino)-2-((((1R,4R)-4-(hydroxymethyl)cyclohexyl)thio)-methyl)quinazolin-4(3H)-one;
2-((((trans)-4-(2-aminoethyl)cyclohexyl)thio)methyl)-8-methylquinazolin-4(3H)-one;
2-(((3-(aminomethyl)cyclobutyl)thio)methyl)-8-methylquinazolin-4(3H)-one;
2-((((trans)-3-(2-aminoethyl)cyclopentyl)thio)methyl)-8-methylquinazolin-4(3H)-one
7-(cyclopentylamino)-5-fluoro-2-(((tetrahydro-2H-pyran-4-yl)thio)methyl)quinazolin-4(3H)-one;
7-(cyclopentylamino)-5-fluoro-2-((((1R,4R)-4-hydroxycyclohexyl)thio)methyl)-quinazolin-4(3H)-one;
7-(cyclopentylamino)-2-(((tetrahydro-2H-pyran-4-yl)thio)methyl)pyrido[2,3-d]pyrimidin-4(3H)-one;
(S)-7-((tetrahydro-2H-pyran-3-yl)amino)-2-(((tetrahydro-2H-pyran-4-yl)thio)methyl)quinazolin-4(3H)-one;
7-(cyclopentylamino)-2-(((tetrahydro-2H-pyran-4-yl)thio)methyl)pyrido[3,2-d]pyrimidin-4(3H)-one;
7-(cyclopentylamino)-2-(((tetrahydro-2H-pyran-4-yl)thio)methyl)pyrido[4,3-d]pyrimidin-4(3H)-one;
2-((azepan-4-ylthio)methyl)-7-(cyclopentylamino)quinazolin-4(3H)-one;
2-(((3-(aminomethyl)cyclopentyl)thio)methyl)-8-methylquinazolin-4(3H)-one;
7-((3-methylisoxazol-5-yl)amino)-2-(((tetrahydro-2H-pyran-4-yl)thio)methyl)quinazolin-4(3H)-one;
(R)-7-((1-(methylsulfonyl)piperidin-3-yl)amino)-2-(((tetrahydro-2H-pyran-4-yl)thio)methyl)quinazolin-4(3H)-one;
7-(cyclobutylamino)-2-((((1R,4R)-4-hydroxycyclohexyl)thio)methyl)quinazolin-4(3H)-one;
7-((1-(methylsulfonyl)azetidin-3-yl)amino)-2-(((tetrahydro-2H-pyran-4-yl)thio)methyl)quinazolin-4(3H)-one;
(R)-7-((1-(methylsulfonyl)piperidin-3-yl)amino)-2-((piperidin-4-ylthio)methyl)quinazolin-4(3H)-one;
7-(cyclopentyloxy)-2-(((tetrahydro-2H-pyran-4-yl)thio)methyl)quinazolin-4(3H)-one;
8-methyl-2-((oxepan-4-ylthio)methyl)quinazolin-4(3H)-one;
7-(cyclopentylamino)-2-((((1R,4R)-4-hydroxycyclohexyl)thio)methyl)-5-(trifluoromethyl)quinazolin-4(3H)-one;
7-(cyclobutylamino)-2-((piperidin-4-ylthio)methyl)quinazolin-4(3H)-one;
(R)-7-((1-(methylsulfonyl)piperidin-3-yl)amino)-2-(((tetrahydro-2H-pyran-4-yl)thio)methyl)pyrido[2,3-d]pyrimidin-4(3H)-one;
7-isobutyl-2-(((tetrahydro-2H-pyran-4-yl)thio)methyl)quinazolin-4(3H)-one;
7-(cyclopentylamino)-5-methyl-2-(((tetrahydro-2H-pyran-4-yl)thio)methyl) quinazolin-4(3H)-one;
cis-4-(((8-methyl-4-oxo-3,4-dihydroquinazolin-2-yl)methyl)thio)cyclohexane-1-carboxamide;
trans-4-(((8-methyl-4-oxo-3,4-dihydroquinazolin-2-yl)methyl)thio)cyclohexane-1-carboxamide;
5-chloro-7-(cyclopentylamino)-2-((piperidin-4-ylthio)methyl)quinazolin-4(3H)-one;
7-(cyclopentylamino)-5-methoxy-2-(((tetrahydro-2H-pyran-4-yl)thio)methyl)quinazolin-4(3H)-one;
methyl 4-(((7-(cyclopentylamino)-4-oxo-3,4-dihydroquinazolin-2-yl)methyl)thio)piperidine-1-carboxylate;
2-((trans)-4-(((8-methyl-4-oxo-3,4-dihydroquinazolin-2-yl)methyl)thio)cyclohexyl) acetamide;
7-(cyclopentylamino)-5-fluoro-2-(((trans-3-fluoropiperidin-4-yl)thio)methyl)quinazolin-4(3H)-one;
7-(cyclopentylamino)-5-fluoro-2-((((3S,4S)-3-fluoropiperidin-4-yl)thio)methyl)quinazolin-4(3H)-one;
7-(cyclopentylamino)-5-fluoro-2-((((3R,4R)-3-fluoropiperidin-4-yl)thio)methyl)quinazolin-4(3H)-one;
7-(cyclopentylamino)-5-fluoro-2-((((cis)-3-fluoropiperidin-4-yl)thio)methyl)quinazolin-4(3H)-one;
7-(cyclopentylamino)-5-fluoro-2-((((3R,4S)-3-fluoropiperidin-4-yl)thio)methyl)quinazolin-4(3H)-one;
7-(cyclopentylamino)-5-fluoro-2-((((3S,4R)-3-fluoropiperidin-4-yl)thio)methyl)quinazolin-4(3H)-one;
7-(cyclopentylamino)-5-fluoro-2-(((1-(2-hydroxyacetyl)piperidin-4-yl)thio)methyl)quinazolin-4(3H)-one;
2-((cyclohexylthio)methyl)-7-(cyclopentylamino)-5-fluoroquinazolin-4(3H)-one;
cis-4-(((7-(cyclopentylamino)-5-fluoro-4-oxo-3,4-dihydroquinazolin-2-yl)methyl)thio)cyclohexane-1-carboxylic acid;
trans-4-(((7-(cyclopentylamino)-5-fluoro-4-oxo-3,4-dihydroquinazolin-2-yl)methyl)thio)cyclohexane-1-carboxylic acid;
trans-4-(((7-(cyclopentylamino)-5-fluoro-4-oxo-3,4-dihydroquinazolin-2-yl)methyl)thio)cyclohexane-1-carboxamide;
7-(cyclopropylmethoxy)-2-(((tetrahydro-2H-pyran-4-yl)thio)methyl)quinazolin-4(3H)-one;
4-(((7-(cyclopentylamino)-5-fluoro-4-oxo-3,4-dihydroquinazolin-2-yl)methyl)thio)-N,N-dimethylpiperidine-1-carboxamide;
2-(((Cis-6-(hydroxymethyl)tetrahydro-2H-pyran-3-yl)thio)methyl)-8-methylquinazolin-4(3H)-one;
7-(cyclopentylamino)-5-fluoro-2-(((trans-3-(trifluoromethyl)piperidin-4-yl)thio)methyl)quinazolin-4(3H)-one;
7-(cyclopentylamino)-5-fluoro-2-(((cis-4-fluoropyrrolidin-3-yl)thio)methyl)quinazolin-4(3H)-one;
7-(cyclopentylamino)-5-(hydroxymethyl)-2-(((tetrahydro-2H-pyran-4-yl)thio)methyl)quinazolin-4(3H)-one;
7-(cyclopentylamino)-5-(fluoromethyl)-2-(((tetrahydro-2H-pyran-4-yl)thio)methyl)quinazolin-4(3H)-one;
7-(cyclopentylamino)-6-fluoro-2-((piperidin-4-ylthio)methyl)quinazolin-4(3H)-one;
7-(cyclopentylamino)-5-fluoro-2-(((trans-2-(trifluoromethyl)piperidin-4-yl)thio)methyl)quinazolin-4(3H)-one;
7-(cyclopentylamino)-5-fluoro-2-(((cis-2-(trifluoromethyl)piperidin-4-yl)thio)methyl)quinazolin-4(3H)-one;
7-(cyclopropylmethoxy)-2-((piperidin-4-ylthio)methyl)pyrido[2,3-d]pyrimidin-4(3H)-one;
7-((cyclobutylmethyl)amino)-6-methoxy-2-((piperidin-4-ylthio)methyl)quinazolin-4(3H)-one;
7-((2,2-difluorocyclopentyl)amino)-5-fluoro-2-(((tetrahydro-2H-pyran-4-yl)thio)methyl)quinazolin-4(3H)-one;
7-(cyclopentylamino)-5,6-difluoro-2-((piperidin-4-ylthio)methyl)quinazolin-4(3H)-one;
5-fluoro-7-((trans-4-morpholinocyclohexyl)amino)-2-(((tetrahydro-2H-pyran-4-yl)thio)methyl)quinazolin-4(3H)-one;
5-fluoro-7-((cis-4-morpholinocyclohexyl)amino)-2-(((tetrahydro-2H-pyran-4-yl)thio)methyl)quinazolin-4(3H)-one;
7-(cyclopropylmethoxy)-5-fluoro-2-(((trans-4-hydroxycyclohexyl)thio)methyl)quinazolin-4(3H)-one;
5-fluoro-7-((tetrahydro-2H-pyran-4-yl)methoxy)-2-(((tetrahydro-2H-pyran-4-yl)thio)methyl)quinazolin-4(3H)-one;
7-(cyclobutylmethoxy)-5-methyl-2-(((tetrahydro-2H-pyran-4-yl)thio)methyl)quinazolin-4(3H)-one;
5-fluoro-2-(((tetrahydro-2H-pyran-4-yl)thio)methyl)-7-((tetrahydrofuran-3-yl)methoxy)quinazolin-4(3H)-one;
(R)-5-fluoro-2-(((tetrahydro-2H-pyran-4-yl)thio)methyl)-7-((tetrahydrofuran-3-yl)methoxy)quinazolin-4(3H)-one.
(S)-5-fluoro-2-(((tetrahydro-2H-pyran-4-yl)thio)methyl)-7-((tetrahydrofuran-3-yl)methoxy)quinazolin-4(3H)-one;
7-(cyclopentylamino)-5-fluoro-2-(((trans-6-fluoroazepan-4-yl)thio)methyl)quinazolin-4(3H)-one;
7-(cyclopentylamino)-5-fluoro-2-(((cis-6-fluoroazepan-4-yl)thio)methyl)quinazolin-4(3H)-one;
2-((((cis)-6-(aminomethyl)tetrahydro-2H-pyran-3-yl)thio)methyl)-7-(cyclopentylamino)-5-fluoroquinazolin-4(3H)-one;
2-(((trans-4-(aminomethyl)-4-fluorocyclohexyl)thio)methyl)-7-(cyclopentylamino)-5-fluoroquinazolin-4(3H)-one;
2-(((cis-4-(aminomethyl)-4-fluorocyclohexyl)thio)methyl)-7-(cyclopentylamino)-5-fluoroquinazolin-4(3H)-one;
6-fluoro-7-((tetrahydro-2H-pyran-4-yl)amino)-2-(((tetrahydro-2H-pyran-4-yl)thio)methyl)quinazolin-4(3H)-one;
7-(cyclopentylamino)-5-fluoro-2-(((1-methylpiperidin-4-yl)thio)methyl)quinazolin-4(3H)-one;
7-(cyclohexylamino)-5-fluoro-2-(((tetrahydro-2H-pyran-4-yl)thio)methyl)quinazolin-4(3H)-one;
7-(cyclohexylamino)-5-fluoro-2-((piperidin-4-ylthio)methyl)quinazolin-4(3H)-one;
7-(cyclohexylamino)-5-fluoro-2-((((1r,4r)-4-hydroxycyclohexyl)thio)methyl)quinazolin-4(3H)-one;
(R)-5-fluoro-7-((1-(methylsulfonyl)piperidin-3-yl)amino)-2-(((tetrahydro-2H-pyran-4-yl)thio)methyl)quinazolin-4(3H)-one;
7-(cyclobutylamino)-5-fluoro-2-(((tetrahydro-2H-pyran-4-yl)thio)methyl)quinazolin-4(3H)-one;
7-((2-cyclopentylethyl)amino)-2-(((tetrahydro-2H-pyran-4-yl)thio)methyl)quinazolin-4(3H)-one;
5-chloro-7-(cyclopentylamino)-2-(((tetrahydro-2H-pyran-4-yl)thio)methyl)quinazolin-4(3H)-one;
7-(cyclopentylamino)-2-(((1-(2,2,2-trifluoroethyl)piperidin-4-yl)thio)methyl)quinazolin-4(3H)-one;
7-(cyclopentylamino)-5-fluoro-2-(((1-(oxetan-3-yl)piperidin-4-yl)thio)methyl)quinazolin-4(3H)-one;
7-((2-(tetrahydro-2H-pyran-4-yl)ethyl)amino)-2-(((tetrahydro-2H-pyran-4-yl)thio)methyl)quinazolin-4(3H)-one;
7-(cyclopentylamino)-5-methyl-2-((piperidin-4-ylthio)methyl)quinazolin-4(3H)-one;
7-(cyclopentylamino)-2-(((1-(2,2-difluoroethyl)piperidin-4-yl)thio)methyl)quinazolin-4(3H)-one;
7-(cyclopentylamino)-2-(((1-(3,3,3-trifluoropropyl)piperidin-4-yl)thio)methyl)quinazolin-4(3H)-one;
2-(((cis-6-(hydroxymethyl)tetrahydro-2H-pyran-2-yl)thio)methyl)-8-methylquinazolin-4(3H)-one;
7-((cyclobutylmethyl)amino)-5-fluoro-2-((piperidin-4-ylthio)methyl)quinazolin-4(3H)-one;
7-(((2,2-difluorocyclopropyl)methyl)amino)-5-fluoro-2-((piperidin-4-ylthio)methyl)quinazolin-4(3H)-one;
7-(cyclopentylamino)-5-fluoro-2-(((1-(2,2,2-trifluoroethyl)piperidin-4-yl)thio)methyl)quinazolin-4(3H)-one;
7-(cyclopentylamino)-2-(((1-(2,2-difluoropropyl)piperidin-4-yl)thio)methyl)quinazolin-4(3H)-one;
7-((cyclopropylmethyl)amino)-5-fluoro-2-((piperidin-4-ylthio)methyl)quinazolin-4(3H)-one;
7-((3,3-difluorocyclopentyl)amino)-5-fluoro-2-((piperidin-4-ylthio)methyl)quinazolin-4(3H)-one;
2-(((trans-4-hydroxycyclohexyl)thio)methyl)-7-(((R)-1-(methylsulfonyl)piperidin-3-yl)amino)quinazolin-4(3H)-one;
(R)-2-(((1-acetylpiperidin-4-yl)thio)methyl)-7-((1-(methylsulfonyl)piperidin-3-yl)amino)quinazolin-4(3H)-one;
5-fluoro-2-(((trans-4-hydroxycyclohexyl)thio)methyl)-7-(((R)-1-(methylsulfonyl)piperidin-3-yl)amino)quinazolin-4(3H)-one;
7-(cyclopentylamino)-2-(((1,1-dioxidotetrahydro-2H-thiopyran-4-yl)thio)methyl)-5-fluoroquinazolin-4(3H)-one;
7-((cyclopropylmethyl)amino)-5-fluoro-2-(((trans-4-hydroxycyclohexyl)thio)methyl)quinazolin-4(3H)-one;
5-fluoro-2-((piperidin-4-ylthio)methyl)-7-(((tetrahydro-2H-pyran-4-yl)methyl)amino)quinazolin-4(3H)-one;
7-(cyclopentylamino)-2-(((1-(1,1-dioxidothietan-3-yl)piperidin-4-yl)thio)methyl)-5-fluoroquinazolin-4(3H)-one;
7-((cyclopropylmethyl)amino)-5-fluoro-2-(((1-(oxetan-3-yl)piperidin-4-yl)thio)methyl)quinazolin-4(3H)-one;
7-(cyclopropylmethoxy)-2-((piperidin-4-ylthio)methyl)quinazolin-4(3H)-one;
7-(cyclopentylamino)-5-fluoro-2-(((1-(2-(methylsulfonyl)ethyl)piperidin-4-yl)thio)methyl)quinazolin-4(3H)-one;
5-fluoro-7-((2-morpholinoethyl)amino)-2-((piperidin-4-ylthio)methyl)quinazolin-4(3H)-one;
7-(cyclopropylmethoxy)-5-fluoro-2-((piperidin-4-ylthio)methyl)quinazolin-4(3H)-one;
7-(cyclopentylamino)-5-fluoro-2-(((1-(2-hydroxy-2-methylpropanoyl)piperidin-4-yl)thio)methyl)quinazolin-4(3H)-one;
7-(cyclobutylmethoxy)-5-fluoro-2-((piperidin-4-ylthio)methyl)quinazolin-4(3H)-one;
7-(cyclopentylamino)-5-fluoro-2-(((1-(pyridin-2-ylmethyl)piperidin-4-yl)thio)methyl)quinazolin-4(3H)-one;
7-(cyclopentylmethoxy)-5-fluoro-2-((piperidin-4-ylthio)methyl)quinazolin-4(3H)-one;
2-(4-(((7-(cyclopentylamino)-5-fluoro-4-oxo-3,4-dihydroquinazolin-2-yl)methyl)thio)piperidin-1-yl)-N-methylacetamide;
7-(((2,2-difluorocyclopropyl)methyl)amino)-5-methyl-2-((piperidin-4-ylthio)methyl)quinazolin-4(3H)-one;
2-(4-(((7-(cyclopentylamino)-5-fluoro-4-oxo-3,4-dihydroquinazolin-2-yl)methyl)thio)piperidin-1-yl)acetonitrile;
2-(trans-4-(((7-(cyclopentylamino)-5-fluoro-4-oxo-3,4-dihydroquinazolin-2-yl)methyl)thio)cyclohexyl)acetamide;
5-fluoro-7-((2-morpholinoethyl)amino)-2-(((tetrahydro-2H-pyran-4-yl)thio)methyl)quinazolin-4(3H)-one;
5-fluoro-2-(((tetrahydro-2H-pyran-4-yl)thio)methyl)-7-((1-(2,2,2-trifluoroethyl)piperidin-4-yl)amino)quinazolin-4(3H)-one;
7-((cyclobutylmethyl)amino)-6-fluoro-2-((piperidin-4-ylthio)methyl)quinazolin-4(3H)-one;
7-(cyclohexylamino)-6-fluoro-2-(((tetrahydro-2H-pyran-4-yl)thio)methyl)quinazolin-4(3H)-one;
7-(cyclopropylmethoxy)-5-fluoro-2-(((tetrahydro-2H-pyran-4-yl)thio)methyl)quinazolin-4(3H)-one;
7-((cyclopropylmethyl)amino)-6-fluoro-2-(((tetrahydro-2H-pyran-4-yl)thio)methyl)quinazolin-4(3H)-one;
7-(cyclopentylamino)-6-fluoro-2-(((trans-4-hydroxycyclohexyl)thio)methyl)quinazolin-4(3H)-one;
7-(cyclopentylamino)-5,6-difluoro-2-(((tetrahydro-2H-pyran-4-yl)thio)methyl)quinazolin-4(3H)-one;
7-(cyclopropylmethoxy)-5-fluoro-2-(((cis-3-fluoropiperidin-4-yl)thio)methyl)quinazolin-4(3H)-one;
7-((cyclobutylmethyl)amino)-2-(((1,1-dioxidotetrahydro-2H-thiopyran-4-yl)thio)methyl)-6-fluoroquinazolin-4(3H)-one;
7-(cyclopropylmethoxy)-5-fluoro-2-(((trans-3-fluoropiperidin-4-yl)thio)methyl)quinazolin-4(3H)-one;
5-fluoro-2-(((tetrahydro-2H-pyran-4-yl)thio)methyl)-7-((1-(3,3,3-trifluoropropyl)piperidin-4-yl)methoxy)quinazolin-4(3H)-one;
7-((1-(2,2-difluoropropyl)piperidin-4-yl)methoxy)-5-fluoro-2-(((tetrahydro-2H-pyran-4-yl)thio)methyl)quinazolin-4(3H)-one;
7-((1-(2,2-difluoroethyl)piperidin-4-yl)methoxy)-5-fluoro-2-(((tetrahydro-2H-pyran-4-yl)thio)methyl)quinazolin-4(3H)-one;
5-fluoro-7-((1-(oxetan-3-yl)piperidin-4-yl)methoxy)-2-(((tetrahydro-2H-pyran-4-yl)thio)methyl)quinazolin-4(3H)-one;
5-fluoro-7-((1-(oxetan-3-yl)piperidin-4-yl)amino)-2-(((tetrahydro-2H-pyran-4-yl)thio)methyl)quinazolin-4(3H)-one;
7-((cyclobutylmethyl)amino)-6-fluoro-2-(((cis-3-fluoropiperidin-4-yl)thio)methyl)quinazolin-4(3H)-one;
7-(cyclobutylmethoxy)-2-(((1,1-dioxidotetrahydro-2H-thiopyran-4-yl)thio)methyl)-5-fluoroquinazolin-4(3H)-one;
5-fluoro-7-((trans-2-fluorocyclopentyl)amino)-2-(((tetrahydro-2H-pyran-4-yl)thio)methyl)quinazolin-4(3H)-one;
5-fluoro-7-isobutoxy-2-(((tetrahydro-2H-pyran-4-yl)thio)methyl)quinazolin-4(3H)-one;
7-(cyclobutylmethoxy)-5-fluoro-2-(((1-(2-hydroxyacetyl)piperidin-4-yl)thio)methyl)quinazolin-4(3H)-one;
7-(cyclobutylmethoxy)-2-(((2,2-dimethyltetrahydro-2H-pyran-4-yl)thio)methyl)-5-fluoroquinazolin-4(3H)-one;
7-(cyclobutylmethoxy)-2-((cyclohexylthio)methyl)-5-fluoroquinazolin-4(3H)-one;
2-((cyclohexylthio)methyl)-7-(cyclopentylamino)-5,6-difluoroquinazolin-4(3H)-one;
trans-4-(((7-(cyclobutylmethoxy)-5-fluoro-4-oxo-3,4-dihydroquinazolin-2-yl)methyl)thio)cyclohexane-1-carboxamide;
7-((1-(2,2-difluoroethyl)piperidin-3-yl)methoxy)-5-fluoro-2-(((tetrahydro-2H-pyran-4-yl)thio)methyl)quinazolin-4(3H)-one;
7-(cyclopentylamino)-5,6-difluoro-2-(((trans-4-hydroxycyclohexyl)thio)methyl)quinazolin-4(3H)-one;
7-(cyclopentylmethoxy)-5-fluoro-2-(((trans-4-hydroxycyclohexyl)thio)methyl)quinazolin-4(3H)-one;
7-((2,2-difluorocyclopropyl)methoxy)-5-fluoro-2-(((tetrahydro-2H-pyran-4-yl)thio)methyl)quinazolin-4(3H)-one;
7-(cyclopentylamino)-2-(((1,1-dioxidotetrahydro-2H-thiopyran-4-yl)thio)methyl)-5,6-difluoroquinazolin-4(3H)-one;
7-((3,3-difluorocyclobutyl)methoxy)-5-fluoro-2-(((tetrahydro-2H-pyran-4-yl)thio)methyl)quinazolin-4(3H)-one;
5-fluoro-2-(((trans-4-hydroxycyclohexyl)thio)methyl)-7-((tetrahydro-2H-pyran-3-yl)methoxy)quinazolin-4(3H)-one;
5-fluoro-7-((tetrahydro-2H-pyran-3-yl)methoxy)-2-(((tetrahydro-2H-pyran-4-yl)thio)methyl)quinazolin-4(3H)-one
5-fluoro-2-(((tetrahydro-2H-pyran-4-yl)thio)methyl)-7-((tetrahydrofuran-2-yl)methoxy)quinazolin-4(3H)-one
(R)-5-fluoro-2-(((tetrahydro-2H-pyran-4-yl)thio)methyl)-7-((tetrahydrofuran-2-yl)methoxy)quinazolin-4(3H)-one;
(S)-5-fluoro-2-(((tetrahydro-2H-pyran-4-yl)thio)methyl)-7-((tetrahydrofuran-2-yl)methoxy)quinazolin-4(3H)-one;
5,6-difluoro-2-(((tetrahydro-2H-pyran-4-yl)thio)methyl)-7-(((tetrahydrofuran-3-yl)methyl)amino)quinazolin-4(3H)-one;
(S)-5,6-difluoro-2-(((tetrahydro-2H-pyran-4-yl)thio)methyl)-7-(((tetrahydrofuran-3-yl)methyl)amino)quinazolin-4(3H)-one;
(R)-5,6-difluoro-2-(((tetrahydro-2H-pyran-4-yl)thio)methyl)-7-(((tetrahydrofuran-3-yl)methyl)amino)quinazolin-4(3H)-one;
5-fluoro-2-((((trans)-4-hydroxycyclohexyl)thio)methyl)-7-((tetrahydrofuran-3-yl)methoxy)quinazolin-4(3H)-one;
5-fluoro-2-((((trans)-4-hydroxycyclohexyl)thio)methyl)-7-(((R)-tetrahydrofuran-3-yl)methoxy)quinazolin-4(3H)-one;
5-fluoro-2-((((trans)-4-hydroxycyclohexyl)thio)methyl)-7-(((S)-tetrahydrofuran-3-yl)methoxy)quinazolin-4(3H)-one;
5-fluoro-7-(((trans)-3-fluoro-1-methylpiperidin-4-yl)methoxy)-2-(((tetrahydro-2H-pyran-4-yl)thio)methyl)quinazolin-4(3H)-one;
5-fluoro-7-(((3S, 4S)-3-fluoro-1-methylpiperidin-4-yl)methoxy)-2-(((tetrahydro-2H-pyran-4-yl)thio)methyl)quinazolin-4(3H)-one;
5-fluoro-7-(((3R, 4R)-3-fluoro-1-methylpiperidin-4-yl)methoxy)-2-(((tetrahydro-2H-pyran-4-yl)thio)methyl)quinazolin-4(3H)-one;
5,6-difluoro-7-(((cis)-3-methoxycyclobutyl)amino)-2-(((tetrahydro-2H-pyran-4-yl)thio)methyl)quinazolin-4(3H)-one;
N-((cis)-4-(((7-(cyclopropylmethoxy)-5-fluoro-4-oxo-3,4-dihydroquinazolin-2-yl)methyl)thio)cyclohexyl)acetamide;
N-((trans)-4-(((7-(cyclopropylmethoxy)-5-fluoro-4-oxo-3,4-dihydroquinazolin-2-yl)methyl)thio)cyclohexyl)acetamide;
7-(((cis)-3-ethoxycyclobutyl)amino)-5,6-difluoro-2-(((tetrahydro-2H-pyran-4-yl)thio)methyl)quinazolin-4(3H)-one;
5-fluoro-2-((((cis)-4-hydroxy-4-methylcyclohexyl)thio)methyl)-7-((tetrahydro-2H-pyran-4-yl)methoxy)quinazolin-4(3H)-one;
7-((1-acetylpiperidin-4-yl)methoxy)-5-fluoro-2-(((tetrahydro-2H-pyran-4-yl)thio)methyl)quinazolin-4(3H)-one;
2-((((trans)-4-(aminomethyl)-4-fluorocyclohexyl)thio)methyl)-7-(cyclobutylmethoxy)-5-fluoroquinazolin-4(3H)-one;
5-fluoro-7-(((3S,4S)-3-fluoro-1-methylpiperidin-4-yl)methoxy)-2-((((trans)-4-hydroxycyclohexyl)thio)methyl)quinazolin-4(3H)-one;
5-fluoro-7-(((3R,4R)-3-fluoro-1-methylpiperidin-4-yl)methoxy)-2-((((trans)-4-hydroxycyclohexyl)thio)methyl)quinazolin-4(3H)-one
7-((cyclopropylmethyl)amino)-5,6-difluoro-2-(((tetrahydro-2H-pyran-4-yl)thio)methyl)quinazolin-4(3H)-one;
5,6-difluoro-7-(((tetrahydro-2H-pyran-4-yl)methyl)amino)-2-(((tetrahydro-2H-pyran-4-yl)thio)methyl)quinazolin-4(3H)-one;
7-((cyclobutylmethyl)amino)-2-(((1,1-dioxidotetrahydro-2H-thiopyran-4-yl)thio)methyl)-5,6-difluoroquinazolin-4(3H)-one;
5-fluoro-7-(((trans)-2-fluorocyclopentyl)amino)-2-((((trans)-4-hydroxycyclohexyl)thio)methyl)quinazolin-4(3H)-one;
5-fluoro-7-(((cis)-2-fluorocyclopentyl)amino)-2-((((trans)-4-hydroxycyclohexyl)thio)methyl)quinazolin-4(3H)-one;
5-fluoro-2-((((trans)-4-hydroxycyclohexyl)thio)methyl)-7-((tetrahydro-2H-pyran-4-yl)methoxy)quinazolin-4(3H)-one;
5-fluoro-7-(oxetan-3-ylmethoxy)-2-(((tetrahydro-2H-pyran-4-yl)thio)methyl)quinazolin-4(3H)-one;
7-((1,4-dioxan-2-yl)methoxy)-5-fluoro-2-(((tetrahydro-2H-pyran-4-yl)thio)methyl)quinazolin-4(3H)-one;
7-((2,2-difluorocyclohexyl)amino)-5-fluoro-2-(((tetrahydro-2H-pyran-4-yl)thio)methyl)quinazolin-4(3H)-one;
5,6-difluoro-7-(((trans)-4-(4-methylpiperazin-1-yl)cyclohexyl)amino)-2-(((tetrahydro-2H-pyran-4-yl)thio)methyl)quinazolin-4(3H)-one;
5,6-difluoro-7-(((cis)-4-(4-methylpiperazin-1-yl)cyclohexyl)amino)-2-(((tetrahydro-2H-pyran-4-yl)thio)methyl)quinazolin-4(3H)-one;
(R)-5,6-difluoro-7-((tetrahydro-2H-pyran-3-yl)amino)-2-(((tetrahydro-2H-pyran-4-yl)thio)methyl)quinazolin-4(3H)-one;
7-(((R)-1-acetylpyrrolidin-3-yl)amino)-5,6-difluoro-2-((((trans)-4-hydroxycyclohexyl)thio)methyl)quinazolin-4(3H)-one;
7-((2,2-difluorocyclopentyl)amino)-5-fluoro-2-((((trans)-4-hydroxycyclohexyl)thio)methyl)quinazolin-4(3H)-one;
7-((1,1-dioxidotetrahydro-2H-thiopyran-4-yl)methoxy)-5-fluoro-2-(((tetrahydro-2H-pyran-4-yl)thio)methyl)quinazolin-4(3H)-one;
5-fluoro-7-(((trans)-3-fluoropiperidin-4-yl)methoxy)-2-(((tetrahydro-2H-pyran-4-yl)thio)methyl)quinazolin-4(3H)-one;
5-chloro-7-((tetrahydro-2H-pyran-4-yl)methoxy)-2-(((tetrahydro-2H-pyran-4-yl)thio)methyl)quinazolin-4(3H)-one;
5,6-difluoro-2-((((trans)-4-hydroxycyclohexyl)thio)methyl)-7-((1-(3,3,3-trifluoropropyl)piperidin-4-yl)amino)quinazolin-4(3H)-one;
7-((5,5-dimethyltetrahydrofuran-3-yl)methoxy)-5-fluoro-2-((((trans)-4-hydroxycyclohexyl)thio)methyl)quinazolin-4(3H)-one;
5-fluoro-2-((((trans)-4-methoxycyclohexyl)thio)methyl)-7-((tetrahydro-2H-pyran-4-yl)methoxy)quinazolin-4(3H)-one;
5-fluoro-2-((((cis)-4-methoxycyclohexyl)thio)methyl)-7-((tetrahydro-2H-pyran-4-yl)methoxy)quinazolin-4(3H)-one;
5-fluoro-2-(((4-methyltetrahydro-2H-pyran-4-yl)thio)methyl)-7-((tetrahydro-2H-pyran-4-yl)methoxy)quinazolin-4(3H)-one;
5-fluoro-7-(((cis)-2-hydroxycyclopentyl)methoxy)-2-(((tetrahydro-2H-pyran-4-yl)thio)methyl)quinazolin-4(3H)-one;
(trans)-4-((5,6-difluoro-4-oxo-2-(((tetrahydro-2H-pyran-4-yl)thio)methyl)-3,4-dihydroquinazolin-7-yl)amino)cyclohexane-1-carbonitrile;
(cis)-4-((5,6-difluoro-4-oxo-2-(((tetrahydro-2H-pyran-4-yl)thio)methyl)-3,4-dihydroquinazolin-7-yl)amino)cyclohexane-1-carbonitrile;
5,6-difluoro-7-(((trans)-3-methoxycyclobutyl)amino)-2-(((tetrahydro-2H-pyran-4-yl)thio)methyl)quinazolin-4(3H)-one;
5,6-difluoro-2-((((trans)-4-hydroxycyclohexyl)thio)methyl)-7-(((cis)-3-methoxycyclobutyl)amino)quinazolin-4(3H)-one;
5-methyl-7-((tetrahydro-2H-pyran-4-yl)methoxy)-2-(((tetrahydro-2H-pyran-4-yl)thio)methyl)quinazolin-4(3H)-one;
5-fluoro-2-((((cis)-4-hydroxycyclohexyl)thio)methyl)-7-((tetrahydro-2H-pyran-4-yl)methoxy)quinazolin-4(3H)-one;
2-(((4,4-difluorocyclohexyl)thio)methyl)-5-fluoro-7-((tetrahydro-2H-pyran-4-yl)methoxy)quinazolin-4(3H)-one;
7-((1-acetylpyrrolidin-3-yl)methoxy)-5-fluoro-2-(((tetrahydro-2H-pyran-4-yl)thio)methyl)quinazolin-4(3H)-one;
7-(2-cyclohexylethyl)-5-fluoro-2-(((tetrahydro-2H-pyran-4-yl)thio)methyl)quinazolin-4(3H)-one;
7-(((1-acetylpiperidin-4-yl)methyl)amino)-5,6-difluoro-2-(((tetrahydro-2H-pyran-4-yl)thio)methyl)quinazolin-4(3H)-one;
5-fluoro-7-(((tetrahydro-2H-pyran-4-yl)methyl)thio)-2-(((tetrahydro-2H-pyran-4-yl)thio)methyl)quinazolin-4(3H)-one;
5-fluoro-7-(((cis)-4-fluoropyrrolidin-3-yl)methoxy)-2-(((tetrahydro-2H-pyran-4-yl)thio)methyl)quinazolin-4(3H)-one;
5-fluoro-7-(((cis)-4-fluoro-1-methylpyrrolidin-3-yl)methoxy)-2-(((tetrahydro-2H-pyran-4-yl)thio)methyl)quinazolin-4(3H)-one;
5-fluoro-2-((((cis)-4-hydroxy-4-methylcyclohexyl)thio)methyl)-7-((tetrahydrofuran-3-yl)methoxy)quinazolin-4(3H)-one;
5,6-difluoro-2-((((cis)-4-hydroxy-4-methylcyclohexyl)thio)methyl)-7-(((cis)-3-methoxycyclobutyl)amino)quinazolin-4(3H)-one;
5-fluoro-7-((tetrahydro-2H-pyran-4-yl)methoxy)-2-((((trans)-4-(trifluoromethoxy)cyclohexyl)thio)methyl)quinazolin-4(3H)-one;
5-bromo-2-(((tetrahydro-2H-pyran-4-yl)thio)methyl)-7-((tetrahydrofuran-3-yl)methoxy)quinazolin-4(3H)-one;
5,6-difluoro-2-((((trans)-4-hydroxycyclohexyl)thio)methyl)-7-(((trans)-4-methoxycyclohexyl)amino)quinazolin-4(3H)-one;
N-((trans)-4-(((7-(cyclopropylmethoxy)-5-fluoro-4-oxo-3,4-dihydroquinazolin-2-yl)methyl)thio)cyclohexyl)propionamide;
5,6-difluoro-2-((((trans)-4-hydroxycyclohexyl)thio)methyl)-7-(((cis)-4-methoxycyclohexyl)amino)quinazolin-4(3H)-one;
N-(4-(((7-(cyclopropylmethoxy)-5-fluoro-4-oxo-3,4-dihydroquinazolin-2-yl)methyl)thio)-1-methylcyclohexyl)acetamide;
5,6-difluoro-2-((((trans)-4-hydroxycyclohexyl)thio)methyl)-7-(((R)-tetrahydro-2H-pyran-3-yl)amino)quinazolin-4(3H)-one;
5-fluoro-2-((((trans)-3-hydroxycyclobutyl)thio)methyl)-7-((tetrahydro-2H-pyran-4-yl)methoxy)quinazolin-4(3H)-one;
4-oxo-2-(((tetrahydro-2H-pyran-4-yl)thio)methyl)-7-((tetrahydrofuran-3-yl)methoxy)-3,4-dihy droquinazoline-5-carbonitrile;
5,6-difluoro-7-(neopentylamino)-2-(((tetrahydro-2H-pyran-4-yl)thio)methyl)quinazolin-4(3H)-one;
5-fluoro-7-(((cis)-3-hydroxy-3-methylcyclobutyl)methoxy)-2-(((tetrahydro-2H-pyran-4-yl)thio)methyl)quinazolin-4(3H)-one;
5-fluoro-7-(((trans)-3-hydroxy-3-methylcyclobutyl)methoxy)-2-(((tetrahydro-2H-pyran-4-yl)thio)methyl)quinazolin-4(3H)-one;
N-((cis)-3-(((5-fluoro-4-oxo-7-((tetrahydro-2H-pyran-4-yl)methoxy)-3,4-dihydroquinazolin-2-yl)methyl)thio)cyclobutyl)acetamide;
5-fluoro-7-(((cis)-3-fluoro-1-methylpiperidin-4-yl)methoxy)-2-(((tetrahydro-2H-pyran-4-yl)thio)methyl)quinazolin-4(3H)-one;
N-((trans)-4-(((5,6-difluoro-7-(((cis)-3-methoxycyclobutyl)amino)-4-oxo-3,4-dihydroquinazolin-2-yl)methyl)thio)cyclohexyl)acetamide;
7-((1-(cyclopropanecarbonyl)piperidin-4-yl)methoxy)-5-fluoro-2-(((tetrahydro-2H-pyran-4-yl)thio)methyl)quinazolin-4(3H)-one;
N-((trans)-4-(((5-fluoro-4-oxo-7-((tetrahydrofuran-3-yl)methoxy)-3,4-dihydroquinazolin-2-yl)methyl)thio)cyclohexyl)acetamide;
N-((trans)-4-(((7-(cyclobutylamino)-5,6-difluoro-4-oxo-3,4-dihydroquinazolin-2-yl)methyl)thio)cyclohexyl)acetamide;
N-((trans)-3-(((5-fluoro-4-oxo-7-((tetrahydro-2H-pyran-4-yl)methoxy)-3,4-dihydroquinazolin-2-yl)methyl)thio)cyclobutyl)acetamide;
7-(1-cyclopentylethoxy)-5-fluoro-2-(((tetrahydro-2H-pyran-4-yl)thio)methyl)-5,6,7,8-tetrahydroquinazolin-4(3H)-one;
N-((trans)-4-(((7-(cyclopropylmethoxy)-5-fluoro-4-oxo-3,4-dihydroquinazolin-2-yl)methyl)thio)cyclohexyl)cyclopropanecarboxamide;
7-((1-acetylpiperidin-4-yl)methoxy)-5-fluoro-2-((((trans)-4-hydroxycyclohexyl)thio)methyl)quinazolin-4(3H)-one;
5-fluoro-7-((1-isobutyrylpiperidin-4-yl)methoxy)-2-(((tetrahydro-2H-pyran-4-yl)thio)methyl)quinazolin-4(3H)-one;
5-fluoro-7-((1-propionylpiperidin-4-yl)methoxy)-2-(((tetrahydro-2H-pyran-4-yl)thio)methyl)quinazolin-4(3H)-one;
5-fluoro-7-(piperidin-4-ylmethoxy)-2-(((tetrahydro-2H-pyran-4-yl)thio)methyl)quinazolin-4(3H)-one;
5,6-difluoro-7-((1-(tetrahydro-2H-pyran-4-yl)ethyl)amino)-2-(((tetrahydro-2H-pyran-4-yl)thio)methyl)quinazolin-4(3H)-one;
7-((1-acetylpiperidin-3-yl)methoxy)-5-fluoro-2-(((tetrahydro-2H-pyran-4-yl)thio)methyl)quinazolin-4(3H)-one;
5,6-difluoro-2-(((tetrahydro-2H-pyran-4-yl)thio)methyl)-7-(((cis)-3-(trifluoromethoxy)cyclobutyl)amino)quinazolin-4(3H)-one;
7-amino-5,6-difluoro-2-(((tetrahydro-2H-pyran-4-yl)thio)methyl)quinazolin-4(3H)-one;
7-(cyclopropylmethoxy)-2-((((trans)-4-(dimethylamino)cyclohexyl)thio)methyl)-5-fluoro-7,8-dihydroquinazolin-4(3H)-one;
5-fluoro-2-((((cis)-3-hydroxycyclobutyl)thio)methyl)-7-((tetrahydro-2H-pyran-4-yl)methoxy)quinazolin-4(3H)-one;
5,6-difluoro-7-((tetrahydro-2H-pyran-4-yl)methoxy)-2-(((tetrahydro-2H-pyran-4-yl)thio)methyl)quinazolin-4(3H)-one;
5,6-difluoro-7-((2-methoxy-2-methylpropyl)amino)-2-(((tetrahydro-2H-pyran-4-yl)thio)methyl)quinazolin-4(3H)-one;
5,6-difluoro-7-((((cis)-3-fluoro-1-methylpiperidin-4-yl)methyl)amino)-2-(((tetrahydro-2H-pyran-4-yl)thio)methyl)quinazolin-4(3H)-one;
7-((1-acetylpiperidin-4-yl)methoxy)-5-fluoro-2-((((cis)-4-hydroxy-4-methylcyclohexyl)thio)methyl)quinazolin-4(3H)-one;
methyl 4-(((5-fluoro-4-oxo-2-(((tetrahydro-2H-pyran-4-yl)thio)methyl)-3,4-dihydroquinazolin-7-yl)oxy)methyl)piperidine-1-carboxylate;
5-fluoro-2-((((trans)-4-hydroxy-4-methylcyclohexyl)thio)methyl)-7-((tetrahydro-2H-pyran-4-yl)methoxy)quinazolin-4(3H)-one;
7-(cyclopentylamino)-5-fluoro-2-((((trans)-3-fluoro-1-methylpiperidin-4-yl)thio)methyl)quinazolin-4(3H)-one;
7-(cyclopentylamino)-5-fluoro-2-((((cis)-3-fluoro-1-methylpiperidin-4-yl)thio)methyl)quinazolin-4(3H)-one;
5-fluoro-7-((4-methylmorpholin-2-yl)methoxy)-2-(((tetrahydro-2H-pyran-4-yl)thio)methyl)quinazolin-4(3H)-one;
5-fluoro-7-((1-methylpiperidin-4-yl)methoxy)-2-(((tetrahydro-2H-pyran-4-yl)thio)methyl)quinazolin-4(3H)-one;
5-fluoro-7-(neopentyloxy)-2-(((tetrahydro-2H-pyran-4-yl)thio)methyl)quinazolin-4(3H)-one;
7-((1-acetylpiperidin-4-yl)methoxy)-5-fluoro-2-((((trans)-4-hydroxy-4-methylcyclohexyl)thio)methyl)quinazolin-4(3H)-one;
5-fluoro-7-((tetrahydro-2H-pyran-4-yl)methoxy)-2-((((cis)-4-(trifluoromethoxy)cyclohexyl)thio)methyl)quinazolin-4(3H)-one;
7-(((1-acetylpiperidin-4-yl)methyl)amino)-5,6-difluoro-2-((((trans)-4-hydroxycyclohexyl)thio)methyl)quinazolin-4(3H)-one;
5,6-difluoro-7-(methylamino)-2-(((tetrahydro-2H-pyran-4-yl)thio)methyl)quinazolin-4(3H)-one;
5-fluoro-2-(((tetrahydro-2H-pyran-4-yl)thio)methyl)-7-(3,3,3-trifluoro-2,2-dimethylpropoxy)quinazolin-4(3H)-one;
7-((1-acetylpiperidin-4-yl)methoxy)-5-fluoro-2-((((cis)-4-hydroxycyclohexyl)thio)methyl)quinazolin-4(3H)-one;
7-((1-acetylpiperidin-4-yl)methoxy)-5-chloro-2-(((tetrahydro-2H-pyran-4-yl)thio)methyl)quinazolin-4(3H)-one;
5-fluoro-7-((1-(2-methoxyacetyl)piperidin-4-yl)methoxy)-2-(((tetrahydro-2H-pyran-4-yl)thio)methyl)quinazolin-4(3H)-one;
5,6-difluoro-7-((((trans)-3-fluoro-1-methylpiperidin-4-yl)methyl)amino)-2-(((tetrahydro-2H-pyran-4-yl)thio)methyl)quinazolin-4(3H)-one;
N-((trans)-4-(((5-fluoro-4-oxo-7-((tetrahydro-2H-pyran-4-yl)methoxy)-3,4-dihydroquinazolin-2-yl)methyl)thio)cyclohexyl)acetamide; and
7-((3,3-difluoro-1-methylpiperidin-4-yl)methoxy)-5-fluoro-2-(((tetrahydro-2H-pyran-4-yl)thio)methyl)quinazolin-4(3H)-one.
   The aforementioned compounds were found to have PARP14 inhibitory activity according to the assay described in Example A.

In some embodiments, L¹ is linked to moiety Q through a covalent bond to ring A.

Ubiquitin ligase binding moieties and linkers are known and well-described in the art, for example: Bondeson, D. P., et al. Nat Chem Biol. 2015 11(8):611-617; An S, et al. EBioMedicine 2018 36:553-562; Paiva S-L. et al, Curr. Op. in Chem. Bio. 2010, 50:111-119; and International Patent Application Publication No. WO 2017/197056, each of which is incorporated by reference in its entirety.

In some embodiments, E is a Von Hippel-Lindau (VHL) E3 ubiquitin ligase binding moiety, a MDM2 E3 ubiquitin ligase binding moiety, a cereblon E3 ubiquitin ligase binding moiety, or an inhibitor of apoptosis proteins (IAP) E3 ubiquitin ligase binding moiety, each of which has an IC₅₀ of less than about 10p M as determined in a binding assay. For example, E is a cereblon E3 ubiquitin ligase binding moiety. E can be a Von Hippel-Lindau (VHL) E3 ubiquitin ligase binding moiety. E can be a MDM2 E3 ubiquitin ligase binding moiety. E can be an IAP E3 ubiquitin ligase binding moiety.

In some embodiments, E comprises a chemical group derived from an imide, a thioimide, an amide, or a thioamide.

In some embodiments, E is thalidomide, lenalidomide, pomalidomide, analogs thereof, isosteres thereof, or derivatives thereof.

In some embodiments, E is a moiety having a structure selected from:

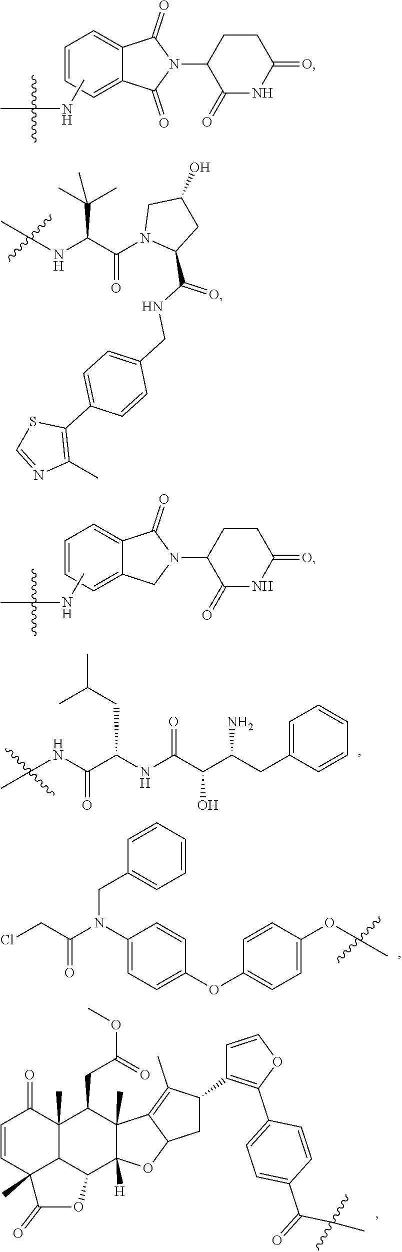

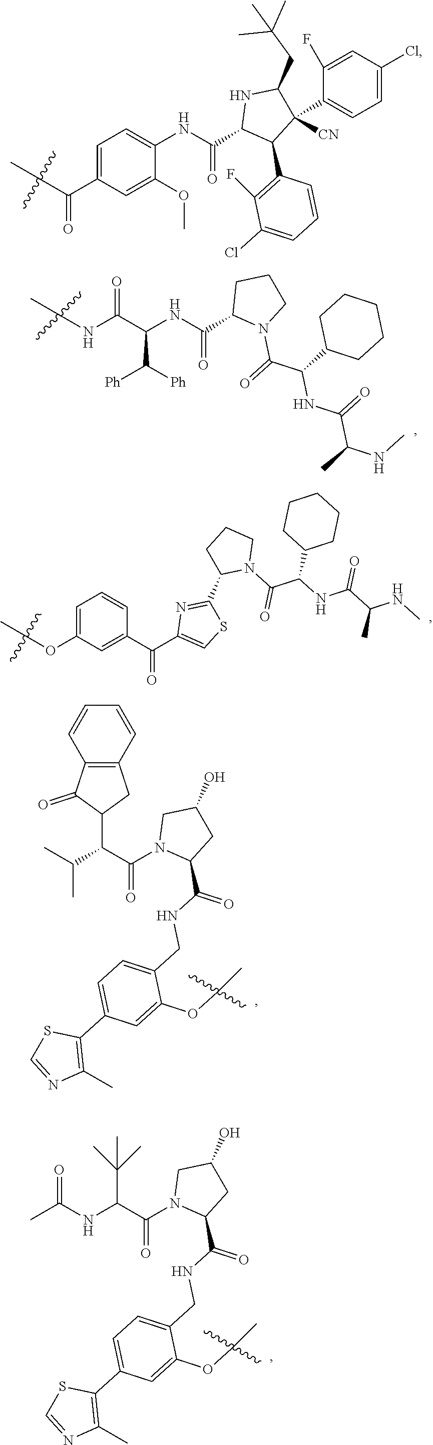

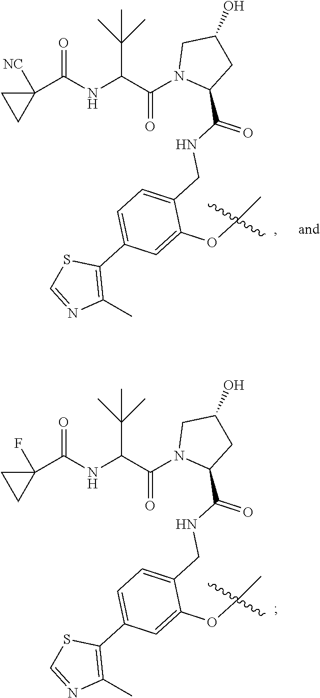 [SMILES: *Oc1cc(-c2scnc2C)ccc1CNC(=O)[C@@H]1C[C@@H](O)CN1C(=O)C(NC(=O)C1(C#N)CC1)C(C)(C)C ; *Oc1cc(-c2scnc2C)ccc1CNC(=O)[C@@H]1C[C@@H](O)CN1C(=O)C(NC(=O)C1(F)CC1)C(C)(C)C]

wherein the wavy lines represent the point of attachment to group L¹.

In some embodiments, E has the following structure:

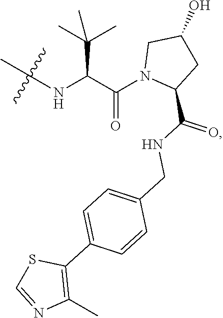 [SMILES: *N[C@H](C(=O)N1C[C@H](O)C[C@H]1C(=O)NCc1ccc(-c2scnc2C)cc1)C(C)(C)C]

wherein the wavy line represents the point of attachment to L¹.

In some embodiments, E has the following structure:

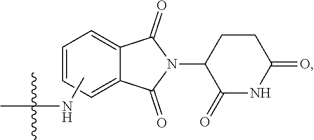

wherein the wavy line represents the point of attachment to L¹.

In some embodiments, E has the following structure:

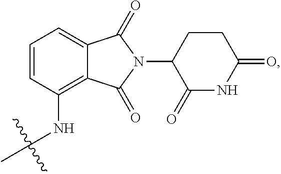 [SMILES: *Nc1cccc2c1C(=O)N(C1CCC(=O)NC1=O)C2=O]

wherein the wavy line represents the point of attachment to L¹.

In some embodiments, linker L¹ is a chain of 1 to 40, 1 to 30, 1 to 25, 1 to 20, 1 to 15, 1 to 10, or 1 to 5 chain atoms, which is optionally substituted with 1-3 R^{q} substituents, and wherein one or more chain carbon atoms of L¹ can be oxidized to form a carbonyl (C═O), and wherein one or more N and S chain atoms can each be optionally oxidized to form an amine oxide, sulfoxide or sulfonyl group; and

each R^{q} is independently selected from OH, CN, —COOH, NH₂, halo, C₁₋₆ haloalkyl, C₁₋₆ alkyl, C₁₋₆ alkoxy, C₁₋₆haloalkoxy, C₁₋₆ alkylthio, phenyl, 5-6 membered heteroaryl, 4-6 membered heterocycloalkyl, C₃₋₆ cycloalkyl, NH(C₁₋₆ alkyl) and N(C₁₋₆ alkyl)₂, wherein the C₁₋₆ alkyl, phenyl, C₃₋₆ cycloalkyl, 4-6 membered heterocycloalkyl, and 5-6 membered heteroaryl of R^{q} are each optionally substituted with halo, OH, CN, —COOH, NH₂, C₁₋₄ alkyl, C₁₋₄ alkoxy, C₁₋₄ haloalkyl, C₁₋₄ haloalkoxy, phenyl, C₃₋₁₀ cycloalkyl, 5- or 6-membered heteroaryl or 4-6 membered heterocycloalkyl. In some embodiments, Rq is independently selected from OH, CN, —COOH, NH₂, halo, C₁₋₆ haloalkyl, C₁₋₆ alkyl, C₁₋₆ alkoxy, C₁₋₆ haloalkoxy, NH(C₁₋₆ alkyl) and N(C₁₋₆ alkyl)₂.

In some embodiments, L¹ has the structure:

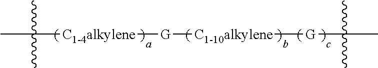

wherein each G is independently selected from —C(O)—, —NR^{G}C(O)—, —NR^{G}—, —O—, —S—, —C(O)O—, —OC(O)NR^{G}—, —NR^{G}C(O)NR^{G}—, —S(O₂)—, or —S(O)NR^{G}—;

each R^{G} is independently selected from H, methyl, and ethyl;

a is 0 or 1;

b is 0 or 1; and

c is 0 or 1, wherein the wavy lines represent points of attachment to moieties Q and E.

In some embodiments, a is 0.

In some embodiments, a is 1.

In some embodiments, b is 0.

In some embodiments, b is 1.

In some embodiments, c is 0.

In some embodiments, c is 1.

In some embodiments, a is 1, b is 1, and c is 1.

In some embodiments, a is 0, b is 1, and c is 0.

In some embodiments, a is 1, b is 1, and c is 0.

In some embodiments, each G is independently selected from —C(O)— and —NR^{G}C(O)—.

In some embodiments, G is —NR^{G}C(O)—.

In some embodiments, R^{G} is H.

In some embodiments linker L¹ is selected from:

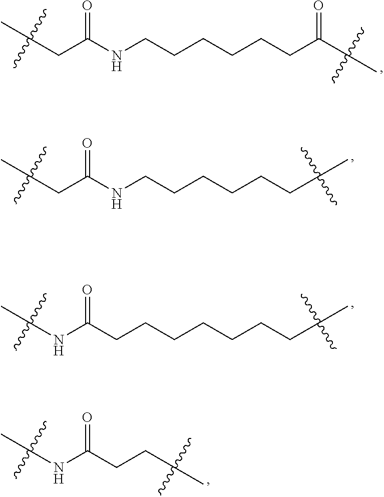 [SMILES: *CC(=O)NCCCCCCC(*)=O ; *CCCCCCNC(=O)C* ; *CCCCCCCC(=O)N* ; *CCC(=O)N*]

and

wherein the wavy lines represent points of attachment to moieties Q and E.

In some embodiments, the compound of the disclosure is a compound of Formula (A2):

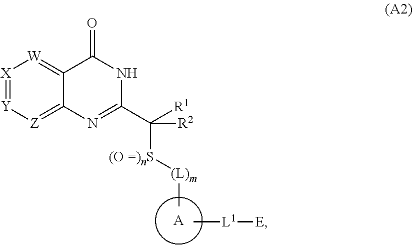

or a pharmaceutically acceptable salt thereof.

In some embodiments, the compound of the disclosure is a compound of Formula (A3):

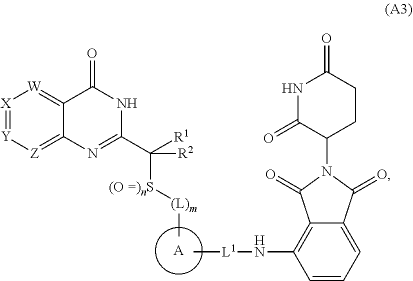

or a pharmaceutically acceptable salt thereof.

In some embodiments, the compound of the disclosure is a compound of Formula (A4):

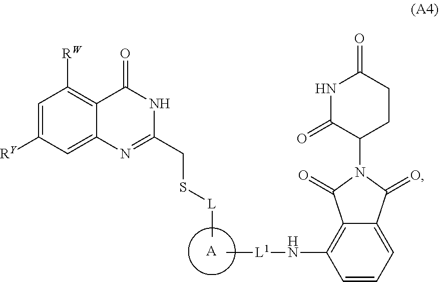

or a pharmaceutically acceptable salt thereof.

In some embodiments, the compound of the disclosure is a compound of Formula (A5):

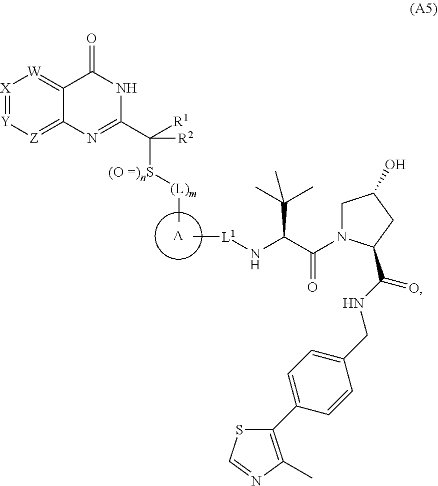

or a pharmaceutically acceptable salt thereof.

In some embodiments, the compound of the disclosure is a compound of Formula (A6):

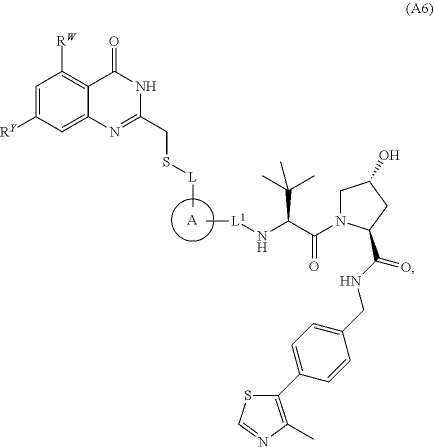

or a pharmaceutically acceptable salt thereof.

In some embodiments, the compound of Formula (A1) is selected from the following:

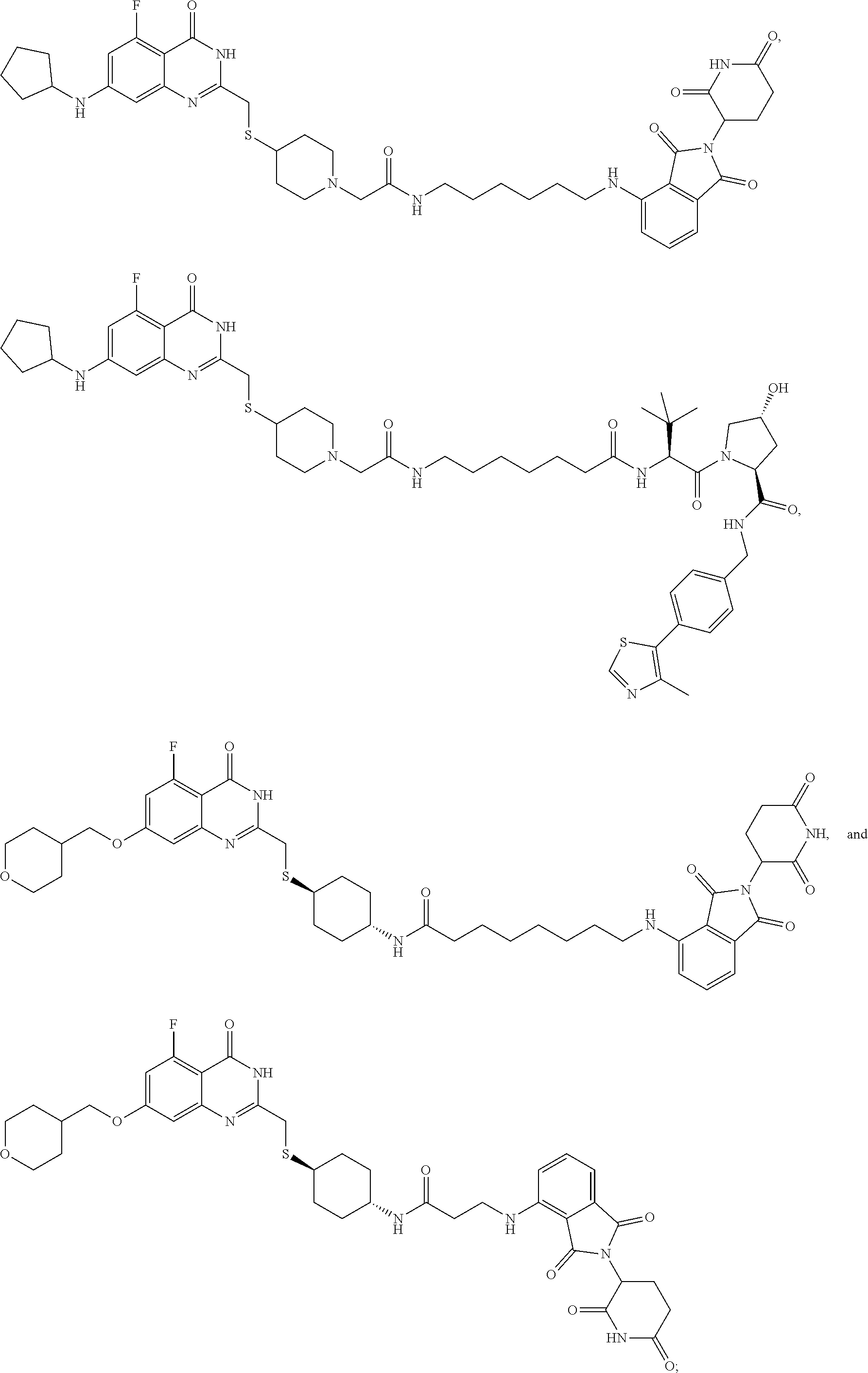 [SMILES: O=C(CN1CCC(SCc2nc3cc(NC4CCCC4)cc(F)c3c(=O)[nH]2)CC1)NCCCCCCNc1cccc2c1C(=O)N(C1CCC(=O)NC1=O)C2=O ; Cc1ncsc1-c1ccc(CNC(=O)[C@@H]2C[C@@H](O)CN2C(=O)[C@@H](NC(=O)CCCCCCNC(=O)CN2CCC(SCc3nc4cc(NC5CCCC5)cc(F)c4c(=O)[nH]3)CC2)C(C)(C)C)cc1 ; O=C1CCC(N2C(=O)c3cccc(NCCCCCCCC(=O)N[C@H]4CC[C@H](SCc5nc6cc(OCC7CCOCC7)cc(F)c6c(=O)[nH]5)CC4)c3C2=O)C(=O)N1 ; "and" ; O=C1CCC(N2C(=O)c3cccc(NCCC(=O)N[C@H]4CC[C@H](SCc5nc6cc(OCC7CCOCC7)cc(F)c6c(=O)[nH]5)CC4)c3C2=O)C(=O)N1]

or a pharmaceutically acceptable salt of any of the aforementioned.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “pyridinyl,” “pyridyl,” or “a pyridine ring” may refer to a pyridin-2-yl, pyridin-3-yl, or pyridin-4-yl ring.

The term “n-membered,” where “n” is an integer, typically describes the number of ring-forming atoms in a moiety where the number of ring-forming atoms is “n”. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

At various places in the present specification, variables defining divalent linking groups may be described. It is specifically intended that each linking substituent include both the forward and backward forms of the linking substituent. For example, —C(O)NR^{G}— includes both

—C(O)NR^{G}— and —NR^{G}C(O)— and is intended to disclose each of the forms individually. Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

For compounds of the invention in which a variable appears more than once, each variable can be a different moiety independently selected from the group defining the variable. For example, where a structure is described having two R groups that are simultaneously present on the same compound, the two R groups can represent different moieties independently selected from the group defined for R.

As used herein, the phrase “optionally substituted” means unsubstituted or substituted.

As used herein, the term “substituted” means that a hydrogen atom is replaced by a non-hydrogen group. It is to be understood that substitution at a given atom is limited by valency.

As used herein, the term “Cᵢ₋ⱼ,” where i and j are integers, employed in combination with a chemical group, designates a range of the number of carbon atoms in the chemical group with i-j defining the range. For example, C₁₋₆ alkyl refers to an alkyl group having 1, 2, 3, 4, 5, or 6 carbon atoms.

As used herein, the term “alkyl,” employed alone or in combination with other terms, refers to a saturated hydrocarbon group that may be straight-chain or branched. In some embodiments, the alkyl group contains 1 to 7, 1 to 6, 1 to 4, or 1 to 3 carbon atoms. Examples of alkyl moieties include, but are not limited to, chemical groups such as methyl, ethyl, n-propyl, isopropyl, n-butyl, isobutyl, sec-butyl, tert-butyl, n-pentyl, 2-methyl-1-butyl, 3-pentyl, n-hexyl, 1,2,2-trimethylpropyl, n-heptyl, and the like. In some embodiments, the alkyl group is methyl, ethyl, or propyl. The term “alkylene” refers to a linking alkyl group.

As used herein, “alkenyl,” employed alone or in combination with other terms, refers to an alkyl group having one or more carbon-carbon double bonds. In some embodiments, the alkenyl moiety contains 2 to 6 or 2 to 4 carbon atoms. Example alkenyl groups include, but are not limited to, ethenyl, n-propenyl, isopropenyl, n-butenyl, sec-butenyl, and the like.

As used herein, “alkynyl,” employed alone or in combination with other terms, refers to an alkyl group having one or more carbon-carbon triple bonds. Example alkynyl groups include, but are not limited to, ethynyl, propyn-1-yl, propyn-2-yl, and the like. In some embodiments, the alkynyl moiety contains 2 to 6 or 2 to 4 carbon atoms.

As used herein, “halo” or “halogen”, employed alone or in combination with other terms, includes fluoro, chloro, bromo, and iodo. In some embodiments, halo is F or C₁.

As used herein, the term “haloalkyl,” employed alone or in combination with other terms, refers to an alkyl group having up to the full valency of halogen atom substituents, which may either be the same or different. In some embodiments, the halogen atoms are fluoro atoms. In some embodiments, the alkyl group has 1 to 6 or 1 to 4 carbon atoms. Example haloalkyl groups include CF₃, C₂F₅, CHF₂, CCl₃, CHCl₂, C₂Cl₅, and the like.

As used herein, the term “alkoxy,” employed alone or in combination with other terms, refers to a group of formula —O-alkyl. Example alkoxy groups include methoxy, ethoxy, propoxy (e.g., n-propoxy and isopropoxy), t-butoxy, and the like. In some embodiments, the alkyl group has 1 to 6 or 1 to 4 carbon atoms.

As used herein, “haloalkoxy,” employed alone or in combination with other terms, refers to a group of formula —O-(haloalkyl). In some embodiments, the alkyl group has 1 to 6 or 1 to 4 carbon atoms. An example haloalkoxy group is —OCF₃.

As used herein, “amino,” employed alone or in combination with other terms, refers to NH₂.

As used herein, the term “alkylamino,” employed alone or in combination with other terms, refers to a group of formula —NH(alkyl). In some embodiments, the alkylamino group has 1 to 6 or 1 to 4 carbon atoms. Example alkylamino groups include methylamino, ethylamino, propylamino (e.g., n-propylamino and isopropylamino), and the like.

As used herein, the term “dialkylamino,” employed alone or in combination with other terms, refers to a group of formula —N(alkyl)₂. Example dialkylamino groups include dimethylamino, diethylamino, dipropylamino (e.g., di(n-propyl)amino and di(isopropyl)amino), and the like. In some embodiments, each alkyl group independently has 1 to 6 or 1 to 4 carbon atoms.

As used herein, the term “cycloalkyl,” employed alone or in combination with other terms, refers to a non-aromatic cyclic hydrocarbon including cyclized alkyl and alkenyl groups. Cycloalkyl groups can include mono- or polycyclic (e.g., having 2, 3, or 4 fused, bridged, or spiro rings) ring systems. Also included in the definition of cycloalkyl are moieties that have one or more aromatic rings (e.g., aryl or heteroaryl rings) fused (i.e., having a bond in common with) to the cycloalkyl ring, for example, benzo derivatives of cyclopentane, cyclohexene, cyclohexane, and the like, or pyrido derivatives of cyclopentane or cyclohexane. Ring-forming carbon atoms of a cycloalkyl group can be optionally substituted by oxo. Cycloalkyl groups also include cycloalkylidenes. The term “cycloalkyl” also includes bridgehead cycloalkyl groups (e.g., non-aromatic cyclic hydrocarbon moieties containing at least one bridgehead carbon, such as admantan-1-yl) and spirocycloalkyl groups (e.g., non-aromatic hydrocarbon moieties containing at least two rings fused at a single carbon atom, such as spiro[2.5]octane and the like). In some embodiments, the cycloalkyl group has 3 to 10 ring members, or 3 to 7 ring members. In some embodiments, the cycloalkyl group is monocyclic or bicyclic. In some embodiments, the cycloalkyl group is monocyclic. In some embodiments, the cycloalkyl group is a C₃₋₇ monocyclic cycloalkyl group. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcamyl, tetrahydronaphthalenyl, octahydronaphthalenyl, indanyl, and the like. In some embodiments, the cycloalkyl group is cyclopropyl, cyclobutyl, cyclopentyl, or cyclohexyl.

As used herein, the term “cycloalkylalkyl,” employed alone or in combination with other terms, refers to a group of formula cycloalkyl-alkyl-. In some embodiments, the alkyl portion has 1 to 4, 1 to 3, 1 to 2, or 1 carbon atom(s). In some embodiments, the alkyl portion is methylene. In some embodiments, the cycloalkyl portion has 3 to 10 ring members or 3 to 7 ring members. In some embodiments, the cycloalkyl group is monocyclic or bicyclic. In some embodiments, the cycloalkyl portion is monocyclic. In some embodiments, the cycloalkyl portion is a C₃₋₇ monocyclic cycloalkyl group.

As used herein, the term “heterocycloalkyl,” employed alone or in combination with other terms, refers to a non-aromatic ring or ring system, which may optionally contain one or more alkenylene or alkynylene groups as part of the ring structure, which has at least one heteroatom ring member independently selected from nitrogen, sulfur, oxygen, and phosphorus. Heterocycloalkyl groups can include mono- or polycyclic (e.g., having 2, 3 or 4 fused, bridged, or spiro rings) ring systems. In some embodiments, the heterocycloalkyl group is a monocyclic or bicyclic group having 1, 2, 3, or 4 heteroatoms independently selected from nitrogen, sulfur and oxygen. Also included in the definition of heterocycloalkyl are moieties that have one or more aromatic rings (e.g., aryl or heteroaryl rings) fused (i.e., having a bond in common with) to the non-aromatic heterocycloalkyl ring, for example, 1,2,3,4-tetrahydro-quinoline and the like. Heterocycloalkyl groups can also include bridgehead heterocycloalkyl groups (e.g., a heterocycloalkyl moiety containing at least one bridgehead atom, such as azaadmantan-1-yl and the like) and spiroheterocycloalkyl groups (e.g., a heterocycloalkyl moiety containing at least two rings fused at a single atom, such as [1,4-dioxa-8-aza-spiro[4.5]decan-N-yl] and the like). In some embodiments, the heterocycloalkyl group has 3 to 10 ring-forming atoms, 4 to 10 ring-forming atoms, or about 3 to 8 ring forming atoms. In some embodiments, the heterocycloalkyl group has 2 to 20 carbon atoms, 2 to 15 carbon atoms, 2 to 10 carbon atoms, or about 2 to 8 carbon atoms. In some embodiments, the heterocycloalkyl group has 1 to 5 heteroatoms, 1 to 4 heteroatoms, 1 to 3 heteroatoms, or 1 to 2 heteroatoms. The carbon atoms or heteroatoms in the ring(s) of the heterocycloalkyl group can be oxidized to form a carbonyl, an N-oxide, or a sulfonyl group (or other oxidized linkage) or a nitrogen atom can be quaternized. In some embodiments, the heterocycloalkyl portion is a C₂₋₇ monocyclic heterocycloalkyl group. In some embodiments, the heterocycloalkyl group is a morpholine ring, pyrrolidine ring, piperazine ring, piperidine ring, tetrahydropyran ring, tetrahyropyridine, azetidine ring, or tetrahydrofuran ring.

As used herein, the term “heterocycloalkylalkyl,” employed alone or in combination with other terms, refers to a group of formula heterocycloalkyl-alkyl-. In some embodiments, the alkyl portion has 1 to 4, 1 to 3, 1 to 2, or 1 carbon atom(s). In some embodiments, the alkyl portion is methylene. In some embodiments, the heterocycloalkyl portion has 3 to 10 ring members, 4 to 10 ring members, or 3 to 7 ring members. In some embodiments, the heterocycloalkyl group is monocyclic or bicyclic. In some embodiments, the heterocycloalkyl portion is monocyclic. In some embodiments, the heterocycloalkyl portion is a C₂₋₇ monocyclic heterocycloalkyl group.

As used herein, the term “aryl,” employed alone or in combination with other terms, refers to a monocyclic or polycyclic (e.g., a fused ring system) aromatic hydrocarbon moiety, such as, but not limited to, phenyl, 1-naphthyl, 2-naphthyl, and the like. In some embodiments, aryl groups have from 6 to 10 carbon atoms or 6 carbon atoms. In some embodiments, the aryl group is a monocyclic or bicyclic group. In some embodiments, the aryl group is phenyl or naphthyl.

As used herein, the term “arylalkyl,” employed alone or in combination with other terms, refers to a group of formula aryl-alkyl-. In some embodiments, the alkyl portion has 1 to 4, 1 to 3, 1 to 2, or 1 carbon atom(s). In some embodiments, the alkyl portion is methylene. In some embodiments, the aryl portion is phenyl. In some embodiments, the aryl group is a monocyclic or bicyclic group. In some embodiments, the arylalkyl group is benzyl.

As used herein, the term “heteroaryl,” employed alone or in combination with other terms, refers to a monocyclic or polycyclic (e.g., a fused ring system) aromatic hydrocarbon moiety, having one or more heteroatom ring members independently selected from nitrogen, sulfur and oxygen. In some embodiments, the heteroaryl group is a monocyclic or a bicyclic group having 1, 2, 3, or 4 heteroatoms independently selected from nitrogen, sulfur and oxygen. Example heteroaryl groups include, but are not limited to, pyridyl, pyrimidinyl, pyrazinyl, pyridazinyl, triazinyl, furyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, purinyl, carbazolyl, benzimidazolyl, indolinyl, pyrrolyl, azolyl, quinolinyl, isoquinolinyl, benzisoxazolyl, imidazo[1,2-b]thiazolyl or the like. The carbon atoms or heteroatoms in the ring(s) of the heteroaryl group can be oxidized to form a carbonyl, an N-oxide, or a sulfonyl group (or other oxidized linkage) or a nitrogen atom can be quaternized, provided the aromatic nature of the ring is preserved. In some embodiments, the heteroaryl group has from 3 to 10 carbon atoms, from 3 to 8 carbon atoms, from 3 to 5 carbon atoms, from 1 to 5 carbon atoms, or from 5 to 10 carbon atoms. In some embodiments, the heteroaryl group contains 3 to 14, 4 to 12, 4 to 8, 9 to 10, or 5 to 6 ring-forming atoms. In some embodiments, the heteroaryl group has 1 to 4, 1 to 3, or 1 to 2 heteroatoms.

As used herein, the term “heteroarylalkyl,” employed alone or in combination with other terms, refers to a group of formula heteroaryl-alkyl-. In some embodiments, the alkyl portion has 1 to 4, 1 to 3, 1 to 2, or 1 carbon atom(s). In some embodiments, the alkyl portion is methylene. In some embodiments, the heteroaryl portion is a monocyclic or bicyclic group having 1, 2, 3, or 4 heteroatoms independently selected from nitrogen, sulfur and oxygen. In some embodiments, the heteroaryl portion has 5 to 10 carbon atoms.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Geometric isomers of olefins, C═N double bonds, and the like can also be present in the compounds described herein, and all such stable isomers are contemplated in the present invention. Cis and trans geometric isomers of the compounds of the present invention may be isolated as a mixture of isomers or as separated isomeric forms.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the invention also include all isotopes of atoms occurring in the intermediates or final compounds. Isotopes include those atoms having the same atomic number but different mass numbers. For example, isotopes of hydrogen include tritium and deuterium. In some embodiments, the compounds of the invention include at least one deuterium atom.

The term “compound,” as used herein, is meant to include all stereoisomers, geometric isomers, tautomers, and isotopes of the structures depicted, unless otherwise specified.

All compounds, and pharmaceutically acceptable salts thereof, can be found together with other substances such as water and solvents (e.g., in the form of hydrates and solvates) or can be isolated.

In some embodiments, the compounds of the invention, or salts thereof, are substantially isolated. By “substantially isolated” is meant that the compound is at least partially or substantially separated from the environment in which it was formed or detected. Partial separation can include, for example, a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof. Methods for isolating compounds and their salts are routine in the art.

The term “small molecule PARP14 targeting moiety” refers to a chemical group that binds to PARP14. The small molecule PARP14 targeting moiety can be a group derived from a compound that inhibits the activity of PARP14. In some embodiments, the small molecule PARP14 targeting moiety inhibits the activity of PARP14 with an IC₅₀ of less than 1 μM in an enzymatic assay (see, e.g., Example A).

The term “Ubiquitin Ligase” refers to a family of proteins that facilitate the transfer of ubiquitin to a specific substrate protein, targeting the substrate protein for degradation.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

Compounds of the invention, including salts thereof, can be prepared using known organic synthesis techniques and can be synthesized according to any of numerous possible synthetic routes.

The reactions for preparing compounds of the invention can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

The expressions, “ambient temperature,” “room temperature,” and “r.t.”, as used herein, are understood in the art, and refer generally to a temperature, e.g. a reaction temperature, that is about the temperature of the room in which the reaction is carried out, for example, a temperature from about 20° C. to about 30° C.

Compounds of the invention can be prepared according to numerous preparatory routes known in the literature. Example synthetic methods for preparing compounds of the invention are provided in the Schemes below.

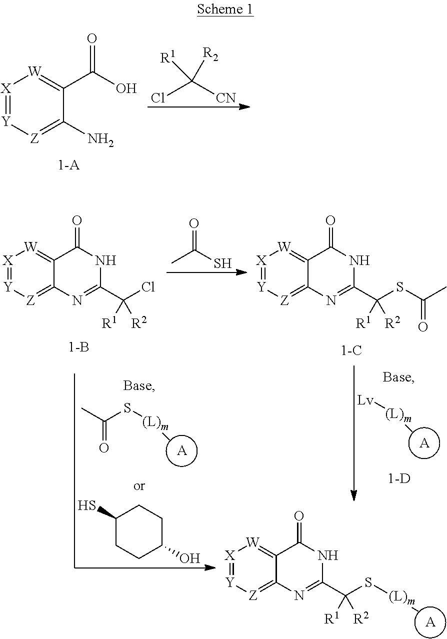

Scheme 1 shows a general synthesis of quinazolinone compounds of the disclosure, corresponding to group Q as defined above. Substituted aminobenzoic acids (1-A), many of which are commercially available or can be made via routes known to one skilled in the art, can be converted to chloromethylquinazolinones (1-B) by treatment with chloroacetonitrile in the presence of a pre-prepared solution of a metal such as sodium in a protic solvent such as methanol at room temperature. The chloro group of 1-B can be converted to a thioacetate (1-C) by treatment with thioacetic acid in a polar solvent such as DMF at room temperature. Introduction of heterocycles (ring A) can be done by treatment with an appropriate electrophile (1-D), where Lv is an appropriate leaving group such as Br, I, methanesulfonate, or para-toluenesulfonate, in the presence of a base such as aqueous sodium hydroxide in a polar solvent such as DMF at elevated temperature such as 90° C. Alternatively, quinazolinones of the invention can be prepared from chloromethylquinazolinones (1-B) by treatment with a thioacetate-substituted heterocycle or trans-4-mercaptocyclohexanol in the presence of a base such as aqueous sodium hydroxide in a polar solvent such as DMF at room temperature.

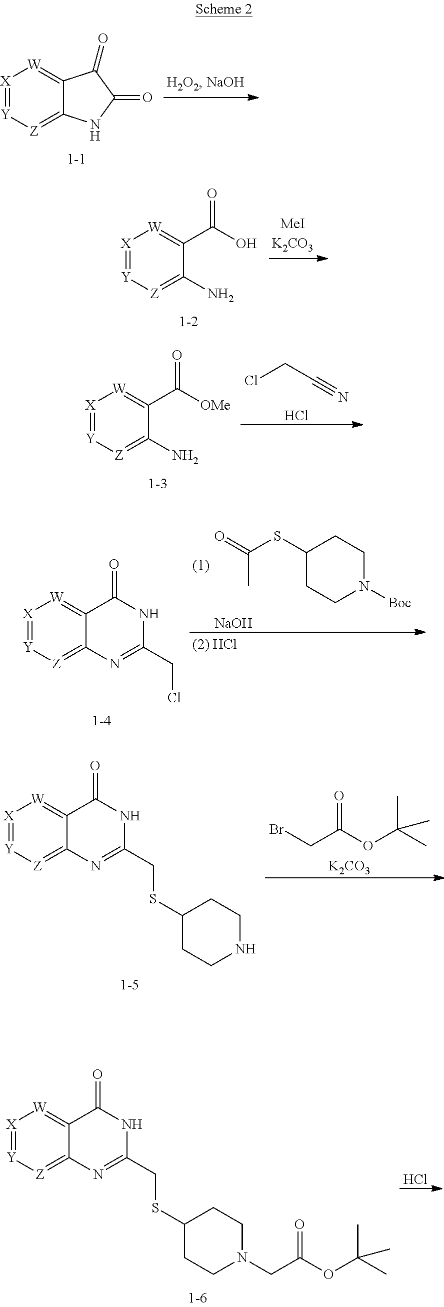

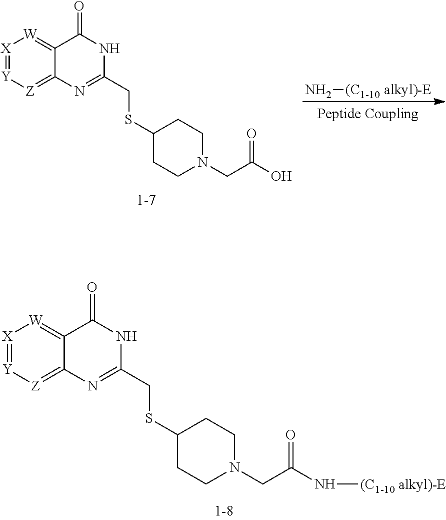

Scheme 2 shows a general synthesis of compounds of the invention. Substituted indoline-2,3-dione (1-1), many of which are commercially available or can be made via routes known to one skilled in the art, can be converted to carboxyclic acids (1-2) by treatment with hydrogen peroxide and a base (e.g., NaOH). Treatment with methyl iodide in the presence of a base (e.g., K₂CO₃) can provide methyl ester (1-3). Treatment with 2-chloroacetonitrile in the presence of acid (e.g., HCl) can provide the corresponding quinazolinone (1-4). Treatment with a thioacetate-substituted heterocycle in the presence of a base (e.g., NaOH) followed by treatment with acid can provide thioether (1-5). Alkylation with a methyl bromoester in the presence of a base (e.g., K₂CO₃) can provide compound (1-6), which can be converted to acid 1-7 by treatment with acid (e.g., HCl). Acid 1-7 can be linked to moiety E under peptide coupling conditions (e.g., EDCI, HOBt, and DIPEA; or HATU, DIPEA) to provide compound 1-8.

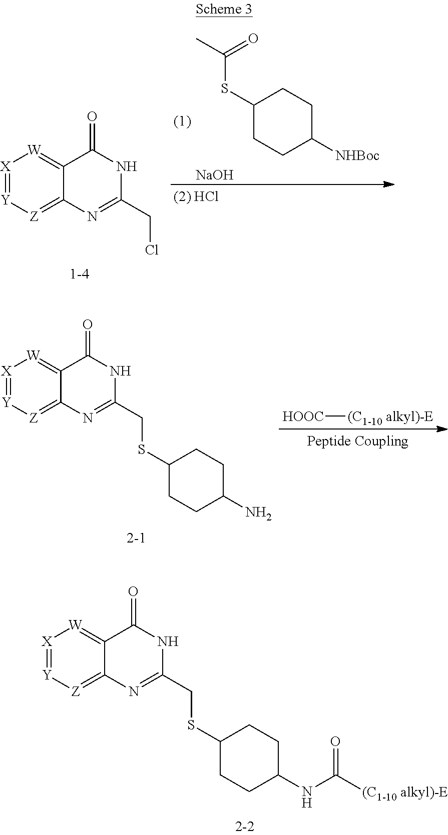

Scheme 3 shows the synthesis of compound 2-2. Treatment of compound 1-4 with a thioacetate-substituted cycloalkyl in the presence of a base (e.g., NaOH) can provide compound 2-1. Compound 2-1 can be linked to moiety E under peptide coupling conditions (e.g., EDCI, HOBt, and DIPEA; or HATU, DIPEA) to provide compound 2-2.

### Methods of Use

Compounds of the present disclosure can bind to both PARP14 and ubiquitin E3 ligase to cause PARP14 degradation, which is useful in the treatment of various diseases including cancer. In some embodiments, the compounds provided herein can degrade PARP14 in a cell, which comprises contacting the cell with the compound or a pharmaceutically acceptable salt or a stereoisomer thereof. In some embodiments, provided herein is a method for degrading PARP14 in a patient, where the method comprises administering to the patient an effective amount of a compound described herein or a pharmaceutically acceptable salt or a stereoisomer thereof. By “degrading PARP14,” it is meant rendering the PARP14 inactive by, for example, altering its structure or breaking down PARP14 into multiple peptide or amino acid fragments.

The compounds of the invention can further inhibit the production of IL-10 in a cell. For example, the present invention relates to methods of inhibiting or decreasing the production of IL-10 in a cell by contacting the cell with a compound of the invention.

The compounds of the invention are useful in the treatment of various diseases associated with abnormal expression or activity of PARP14. For example, the compounds of the invention are useful in the treatment of cancer. In some embodiments, the cancers treatable according to the present invention include hematopoietic malignancies such as leukemia and lymphoma. Example lymphomas include Hodgkin's or non-Hodgkin's lymphoma, multiple myeloma, B-cell lymphoma (e.g., diffuse large B-cell lymphoma (DLBCL)), chronic lymphocytic lymphoma (CLL), T-cell lymphoma, hairy cell lymphoma, and Burkett's lymphoma. Example leukemias include acute lymphocytic leukemia (ALL), acute myelogenous leukemia (AML), chronic lymphocytic leukemia (CLL), and chronic myelogenous leukemia (CML).

Other cancers treatable by the administration of the compounds of the invention include liver cancer (e.g., hepatocellular carcinoma), bladder cancer, bone cancer, glioma, breast cancer, cervical cancer, colon cancer, endometrial cancer, epithelial cancer, esophageal cancer, Ewing's sarcoma, pancreatic cancer, gallbladder cancer, gastric cancer, gastrointestinal tumors, head and neck cancer, intestinal cancers, Kaposi's sarcoma, kidney cancer, laryngeal cancer, liver cancer (e.g., hepatocellular carcinoma), lung cancer, prostate cancer, rectal cancer, skin cancer, stomach cancer, testicular cancer, thyroid cancer, and uterine cancer.

In some embodiments, the cancer treatable by administration of the compounds of the invention is multiple myeloma, DLBCL, hepatocellular carcinoma, bladder cancer, esophageal cancer, head and neck cancer, kidney cancer, prostate cancer, rectal cancer, stomach cancer, thyroid cancer, uterine cancer, breast cancer, glioma, follicular lymphoma, pancreatic cancer, lung cancer, colon cancer, or melanoma.

The compounds of the invention may also have therapeutic utility in PARP14-related disorders in disease areas such as cardiology, virology, neurodegeneration, inflammation, and pain, particularly where the diseases are characterized by overexpression or increased activity of PARP14.

In some embodiments, the compounds of the invention are useful in the treatment of an inflammatory disease. In some embodiments, the inflammatory diseases treatable according to the present invention include inflammatory bowel diseases (e.g., Crohn's disease or ulcerative colitis), inflammatory arthritis, inflammatory demyelinating disease, psoriasis, allergy and asthma sepsis, allergic airway disease (e.g., asthma), and lupus.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” PARP14 or “contacting” a cell with a compound of the invention includes the administration of a compound of the present invention to an individual or patient, such as a human, having PARP14, as well as, for example, introducing a compound of the invention into a sample containing a cellular or purified preparation containing PARP14.

As used herein, the term “individual” or “patient,” used interchangeably, refers to mammals, and particularly humans.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease in an individual who is experiencing or displaying the pathology or symptomatology of the disease (i.e., arresting further development of the pathology and/or symptomatology), or 2) ameliorating the disease in an individual who is experiencing or displaying the pathology or symptomatology of the disease (i.e., reversing the pathology and/or symptomatology).

As used herein the term “preventing” or “prevention” refers to preventing the disease in an individual who may be predisposed to the disease but does not yet experience or display the pathology or symptomatology of the disease.

### Combination Therapy

One or more additional pharmaceutical agents or treatment methods such as, for example, chemotherapeutics or other anti-cancer agents, immune enhancers, immunosuppressants, immunotherapies, radiation, anti-tumor and anti-viral vaccines, cytokine therapy (e.g., IL2, GM-CSF, etc.), and/or kinase (tyrosine or serine/threonine), epigenetic or signal transduction inhibitors can be used in combination with the compounds of the present invention. The agents can be combined with the present compounds in a single dosage form, or the agents can be administered simultaneously or sequentially as separate dosage forms.

Suitable agents for use in combination with the compounds of the present invention for the treatment of cancer include chemotherapeutic agents, targeted cancer therapies, immunotherapies or radiation therapy. Compounds of this invention may be effective in combination with anti-hormonal agents for treatment of breast cancer and other tumors. Suitable examples are anti-estrogen agents including but not limited to tamoxifen and toremifene, aromatase inhibitors including but not limited to letrozole, anastrozole, and exemestane, adrenocorticosteroids (e.g. prednisone), progestins (e.g. megastrol acetate), and estrogen receptor antagonists (e.g. fulvestrant). Suitable anti-hormone agents used for treatment of prostate and other cancers may also be combined with compounds of the present invention. These include anti-androgens including but not limited to flutamide, bicalutamide, and nilutamide, luteinizing hormone-releasing hormone (LHRH) analogs including leuprolide, goserelin, triptorelin, and histrelin, LHRH antagonists (e.g. degarelix), androgen receptor blockers (e.g. enzalutamide) and agents that inhibit androgen production (e.g. abiraterone).

Angiogenesis inhibitors may be efficacious in some tumors in combination with FGFR inhibitors. These include antibodies against VEGF or VEGFR or kinase inhibitors of VEGFR. Antibodies or other therapeutic proteins against VEGF include bevacizumab and aflibercept. Inhibitors of VEGFR kinases and other anti-angiogenesis inhibitors include but are not limited to sunitinib, sorafenib, axitinib, cediranib, pazopanib, regorafenib, brivanib, and vandetanib

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide (Cytoxan™), ifosfamide, melphalan, chlorambucil, pipobroman, triethylene-melamine, triethylenethiophosphoramine, busulfan, carmustine, lomustine, streptozocin, dacarbazine, and temozolomide.

Other anti-cancer agent(s) include antibody therapeutics to costimulatory molecules such as CTLA-4, 4-1BB, PD-1, and PD-L1, or antibodies to cytokines (IL-10, TGF-β, etc.). Exemplary cancer immunotherapy antibodies include alemtuzumab, ipilimumab, nivolumab, ofatumumab and rituximab.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, N.J.), the disclosure of which is incorporated herein by reference as if set forth in its entirety.

### Pharmaceutical Formulations and Dosage Forms

When employed as pharmaceuticals, the compounds of the invention can be administered in the form of pharmaceutical compositions. A pharmaceutical composition refers to a combination of a compound of the invention, or its pharmaceutically acceptable salt, and at least one pharmaceutically acceptable carrier. These compositions can be prepared in a manner well known in the pharmaceutical art, and can be administered by a variety of routes, depending upon whether local or systemic treatment is desired and upon the area to be treated. Administration may be oral, topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, or parenteral.

This invention also includes pharmaceutical compositions which contain, as the active ingredient, one or more of the compounds of the invention above in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

The compositions can be formulated in a unit dosage form. The term “unit dosage form” refers to a physically discrete unit suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid pre-formulation composition containing a homogeneous mixture of a compound of the present invention. When referring to these pre-formulation compositions as homogeneous, the active ingredient is typically dispersed evenly throughout the composition so that the composition can be readily subdivided into equally effective unit dosage forms such as tablets, pills and capsules. This solid pre-formulation is then subdivided into unit dosage forms of the type described above containing from, for example, 0.1 to about 500 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compounds of the invention can also be formulated in combination with one or more additional active ingredients which can include any pharmaceutical agent such as anti-viral agents, anti-cancer agents, vaccines, antibodies, immune enhancers, immune suppressants, anti-inflammatory agents and the like.

### EXAMPLES

Equipment: ¹H NMR Spectra were recorded at 400 MHz using a Bruker AVANCE 400 MHz spectrometer. NMR interpretation was performed using MestReC or MestReNova software to assign chemical shift and multiplicity. In cases where two adjacent peaks of equal or unequal height were observed, these two peaks may be labeled as either a multiplet or as a doublet. In the case of a doublet, a coupling constant using this software may be assigned. In any given example, one or more protons may not be observed due to obscurity by water and/or solvent peaks. LCMS equipment and conditions are as follows:
LC: Agilent Technologies 1290 series, Binary Pump, Diode Array Detector. Agilent Poroshell 120 EC-C18, 2.7 μm, 4.6×50 mm column. Mobile phase: A: 0.05% Formic acid in water (v/v), B: 0.05% Formic acid in ACN (v/v). Flow Rate: 1 mL/min at 25° C. Detector: 214 nm, 254 nm. Gradient stop time, 10 min. Timetable:

| T (min) | A (%) | B (%) |
|---|---|---|
| 0.0 | 90 | 10 |
| 0.5 | 90 | 10 |
| 8.0 | 10 | 90 |
| 10.0 | 0 | 100 |

MS: G6120A, Quadrupole LC/MS, Ion Source: ES-API, TIC: 70-1000 m/z, Fragmentor: 60, Drying gas flow: 10 L/min, Nebulizer pressure: 35 psi, Drying gas temperature: 350° C., Vcap: 3000V.
Sample preparation: samples were dissolved in ACN or methanol at 1-10 mg/mL, then filtered through a 0.22 μm filter membrane. Injection volume: 1˜10 μL.
Definitions: ACN (acetonitrile); Boc (tert-butoxycarbonyl); Boc₂O (di-tert-butyl dicarbonate); CDCl₃ (deuterated chloroform); CD₃OD (deuterated methanol); conc. (concentrated); DCM (dichloromethane); DIPEA (N,N-diisopropylethylamine); DMF (N,N-dimethylformamide); DMSO (dimethylsulfoxide); DMSO-d₆ (deuterated dimethylsulfoxide); EDCI (1-ethyl-3-(3-dimethylaminopropyl)carbodiimide); ES-API (electrospray atmospheric pressure ionization); EtOAc (ethyl acetate); g (gram); h (hour); HATU (1-[bis(dimethylamino)methylene]-1H-1,2,3-triazolo[4,5-b]pyridinium 3-oxide hexafluorophosphate); HOBt (hydroxybenzotriazole); ¹H NMR (proton nuclear magnetic resonance); HPLC (high-performance liquid chromatography); Hz (hertz); KSAc (potassium thioacetate); L (litre); LCMS (liquid chromatography-mass spectrometry); M (molar); MeOH (methanol); mg (milligrams); MHz (megahertz); min (minutes); mL (millilitres), mmol (millimoles); MsCl (methanesulfonyl chloride); NMP (N-methyl-2-pyrrolidone); ppm (parts per million); RT (room temperature); TFA (trifluoroacetic acid); THF (tetrahydrofuran); TIC (total ion chromatogram); TLC (thin layer chromatography); v/v (volume/volume).

### Synthesis of Intermediates

### Int-1: tert-Butyl 4-(acetylthio)piperidine-1-carboxylate

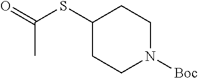 [SMILES: CC(=O)SC1CCN(C(=O)OC(C)(C)C)CC1]

To a solution of tert-butyl 4-bromopiperidine-1-carboxylate (50 g, 189.3 mmol) in DMF (200 mL) was added KSAc (25.9 g, 227.1 mmol). The mixture was stirred at 25° C. for 24 h under a N₂ atmosphere. The reaction mixture was poured into water (300 mL) and extracted with EtOAc (300 mL×3). The combined organic layers were washed with water (500 mL×3), dried over Na₂SO₄ and concentrated to afford the title compound (47.2 g, 96.1%) as a brown oil. ¹H NMR (400 MHz, CDCl₃) δ 3.87-3.84 (m, 2H), 3.64-3.57 (m, 1H), 3.08-3.02 (m, 2H), 2.31 (s, 3H), 1.92-1.87 (m, 2H), 1.58-1.45 (m, 2H), 1.45 (s, 9H).

### Int-2: 4-(6-Aminohexylamino)-2-(2,6-dioxo-3-piperidyl)isoindoline-1,3-dione Hydrochloride

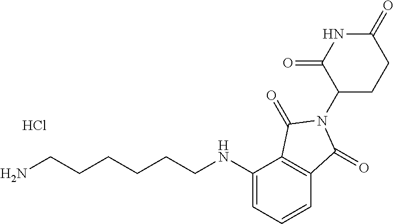 [SMILES: NCCCCCCNc1cccc2c1C(=O)N(C1CCC(=O)NC1=O)C2=O ; Cl]

### Step 1: tert-Butyl N-[6-[[2-(2,6-dioxo-3-piperidyl)-1,3-dioxo-isoindolin-4-yl]amino]hexyl]carbamate

To a solution of 2-(2,6-dioxo-3-piperidyl)-4-fluoro-isoindoline-1,3-dione (300 mg, 1.1 mmol; purchased from Sigma Aldrich) and tert-butyl N-(6-aminohexyl)carbamate (258 mg, 1.2 mmol) in NMP (12 mL) was added DIPEA (280 mg, 2.2 mmol) and the mixture was stirred at 90° C. overnight. The mixture was diluted with water (5 mL) and extracted with EtOAc (20 mL×3). The combined organic layers were dried over Na₂SO₄ and concentrated under reduced pressure. The residue was purified by preparative-TLC (DCM:MeOH, 30:1, v/v) to afford the title compound (200 mg, 39%) as a green solid. LCMS: [M+Na]⁺ 495.2.

### Step 2: 4-(6-Aminohexylamino)-2-(2,6-dioxo-3-piperidyl)isoindoline-1,3-dione Hydrochloride

To a solution of tert-butyl N-[6-[[2-(2,6-dioxo-3-piperidyl)-1,3-dioxo-isoindolin-4-yl]amino]hexyl]carbamate (200 mg, 0.42 mmol) was added HCl/EtOAc (10 mL, 18 mmol) and the mixture was stirred at RT overnight. The mixture was concentrated under reduced pressure and washed with EtOAc to afford the title compound (140 mg, 81%) as a green solid. LCMS: [M+H]⁺ 373.2.

### Int-3: S-((1r,4r)-4-((tert-Butoxycarbonyl)amino)cyclohexyl) Ethanethioate

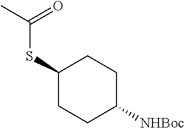 [SMILES: CC(=O)S[C@H]1CC[C@H](NC(=O)OC(C)(C)C)CC1]

### Step 1: (1s,4s)-4-((tert-Butoxycarbonyl)amino)cyclohexyl Methanesulfonate

To a solution of tert-butyl ((1s,4s)-4-hydroxycyclohexyl)carbamate (5 g, 23.2 mmol) and triethylamine (4.7 g, 46.5 mmol) in DCM (25 mL) at RT under a N₂ atmosphere was added MsCl (4.0 g, 34.8 mmol) and the mixture was stirred for 2 h. The mixture was diluted with water (20 mL), extracted with EtOAc (30 mL×3), and the combined organic layers were dried over Na₂SO₄ and concentrated under reduced pressure. The residue was purified by column chromatography (DCM:MeOH, 15:1, v/v) to afford 6.4 g of the title compound. ¹H NMR (400 MHz, DMSO-d₆) δ 6.90-6.82 (m, 1H), 4.8 (br s, 1H), 3.35-3.32 (m, 1H), 3.15 (s, 3H), 1.94-1.88 (m, 2H), 1.71-1.59 (m, 4H), 1.51-1.46 (m, 2H), 1.39 (s, 9H).

### Step 2: S-((1r,4r)-4-((tert-Butoxycarbonyl)amino)cyclohexyl) Ethanethioate

To a solution of (1s,4s)-4-((tert-butoxycarbonyl)amino)cyclohexyl methanesulfonate (3.3 g, 11.3 mmol) in DMF (15 mL) was added KSAc (1.9 g, 16.9 mmol), and the mixture was stirred at 70° C. for 2 h under a N₂ atmosphere. The residue was diluted with water (20 mL) and extracted with EtOAc (30 mL×3) The combined organic layers were dried over Na₂SO₄ and concentrated under reduced pressure. The residue was purified by column chromatography (Petroleum ether:EtOAc, 15:1, v/v) to afford the title compound (0.9 g, 29% yield) as a brown solid. LCMS: [M+H]⁺ 274.3.

### Int-4: 8-[[2-(2,6-Dioxo-3-piperidyl)-1,3-dioxo-isoindolin-4-yl]amino]octanoic Acid

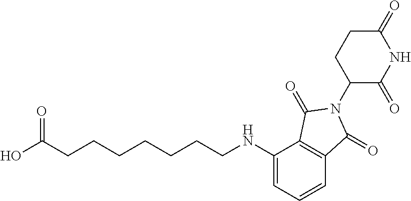 [SMILES: O=C(O)CCCCCCCNc1cccc2c1C(=O)N(C1CCC(=O)NC1=O)C2=O]

To a solution of 2-(2,6-dioxo-3-piperidyl)-4-fluoro-isoindoline-1,3-dione (100 mg, 0.36 mmol; purchased from Sigma Aldrich) in NMP (2 mL) was added 8-aminooctanoic acid (69 mg, 0.43 mmol) and DIPEA (234 mg, 1.8 mmol). The mixture was stirred at 90° C. overnight. The mixture was diluted with 10 mL 1 N HCl and extracted with EtOAc (20 mL×3). The combined organic layers were dried over Na₂SO₄ and concentrated under reduced pressure. The residue was purified by column chromatography (DCM:MeOH, 40:1 to 20:1, v/v) to afford the title compound (50 mg, 33%) as a yellow solid. LCMS: [M+H]⁺ 416.2.

### Int-5: 3-[[2-(2,6-Dioxo-3-piperidyl)-1,3-dioxo-isoindolin-4-yl]amino]propanoic Acid

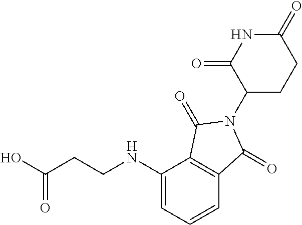 [SMILES: O=C(O)CCNc1cccc2c1C(=O)N(C1CCC(=O)NC1=O)C2=O]

To a solution of 2-(2,6-dioxo-3-piperidyl)-4-fluoro-isoindoline-1,3-dione (200 mg, 0.72 mmol; purchased from Sigma Aldrich) in NMP (4 mL) was added 3-aminopropanoic acid (97 mg, 1.1 mmol) and DIPEA (467 mg, 3.6 mmol). The mixture was stirred at 90° C. overnight. The mixture was diluted with 1 N HCl (10 mL) and extracted with EtOAc (10 mL×3). The combined organic layers were dried over Na₂SO₄ and concentrated under reduced pressure. The residue was purified by preparative-TLC (DCM:MeOH=20:1, v/v) to afford the title compound (60 mg, 24%) as a yellow solid. LCMS: [M+H]⁺ 346.1.

### Example 1: 2-(4-(((7-(Cyclopentylamino)-5-fluoro-4-oxo-3,4-dihydroquinazolin-2-yl)methyl)thio)piperidin-1-yl)-N-(6-((2-(2,6-dioxopiperidin-3-yl)-1,3-dioxoisoindolin-4-yl)amino)hexyl)acetamide Trifluoroacetate

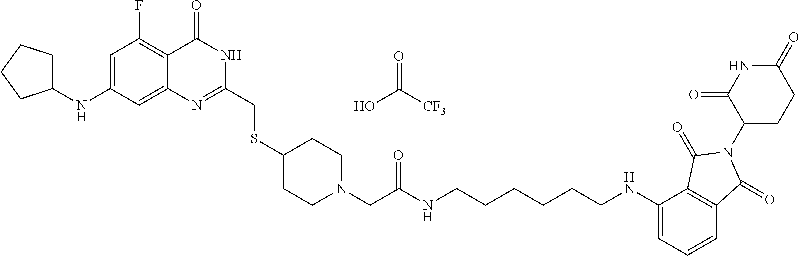 [SMILES: O=C(CN1CCC(SCc2nc3cc(NC4CCCC4)cc(F)c3c(=O)[nH]2)CC1)NCCCCCCNc1cccc2c1C(=O)N(C1CCC(=O)NC1=O)C2=O ; O=C(O)C(F)(F)F]

### Step 1: 2-Amino-4,6-difluoro-benzoic Acid

To a suspension of 4,6-difluoroindoline-2,3-dione (25.0 g, 136.5 mmol) in 1 N NaOH (137 mL, 137 mmol) at 0° C. was added H₂O₂ (30%, 45.0 mL) dropwise and the mixture was allowed to warm to RT and stirred for 5 h. The mixture was poured into water (200 mL) and adjusted to pH 6-7 with 1 N HCl. The precipitate was collected by filtration, washed with water, and dried under vacuum to give the title compound (21.7 g, 92%) as a yellow solid. LCMS: [M+H]⁺ 174.1.

### Step 2: Methyl 2-amino-4,6-difluoro-benzoate

To a suspension of 2-amino-4,6-difluoro-benzoic acid (94.0 g, 543.0 mmol) and K₂CO₃ (112.6 g, 814.5 mmol) in DMF (1 L) was added iodomethane (92.5 g, 651.6 mmol) dropwise under N₂ atmosphere, and the mixture was stirred at 20° C. for 2 h. The mixture was quenched with water (3.5 L), and stirred at 20° C. for 30 min. The suspension was filtered. The cake was washed with 1 L of a solution of a 20:1 petroleum ether:EtOAc and dried under vacuum to afford the title compound (89 g, 88%) as brown solid. LCMS: [M+H]⁺ 188.1.

### Step 3: Methyl 2-amino-4-(cyclopentylamino)-6-fluorobenzoate

To a solution of methyl 2-amino-4,6-difluorobenzoate (3 g, 16.0 mmol, 1.0 eq) in DMSO (5 mL) was added cyclopentanamine (2.73 g, 32.0 mmol, 2.0 eq) and the mixture was heated at 80° C. overnight. The mixture was cooled to RT, diluted with water (5 mL) and extracted with DCM (40 mL×2). The combined organic layers were dried over Na₂SO₄ and concentrated under reduced pressure. The residue was purified by column chromatography (Petroleum ether:DCM, 40:1, v/v to Petroleum ether:EtOAc, 30:1 to 20:1, v/v) to afford the title compound (863 mg, 21%) as a red solid. LCMS: [M+H]⁺ 253.1.

### Step 4: 2-(Chloromethyl)-7-(cyclopentylamino)-5-fluoroquinazolin-4(3H)-one

A mixture of methyl 2-amino-4-(cyclopentylamino)-6-fluorobenzoate (48.0 g, 190.3 mmol) and 2-chloroacetonitrile (60.2 mL, 951.3 mmol) in 4 N HCl in dioxane (240.0 mL, 960 mmol) was heated at 100° C. in a sealed tube overnight. The mixture was diluted with 770 mL of a 10:1 solution of petroleum ether:EtOAc and stirred at room temperature for 1 h. The suspension was filtered, and the cake was dried under vacuum to afford the title compound (56.0 g, 99.5% yield) as brown solid. LCMS: [M+H]⁺ 296.1.

### Step 5: Tert-Butyl 4-(((7-(cyclopentylamino)-5-fluoro-4-oxo-3,4-dihydroquinazolin-2-yl)methyl) thio)piperidine-1-carboxylate

To a solution of 2-(chloromethyl)-7-(cyclopentylamino)-5-fluoro-3H-quinazolin-4-one (1 g, 3.4 mmol) and tert-butyl 4-acetylsulfanylpiperidine-1-carboxylate (1.1 g, 4.1 mmol) in THF (20 mL) was added 2 M NaOH (6.8 mL, 13.5 mmol) and the mixture was stirred at RT overnight under a nitrogen atmosphere. The mixture was diluted with water (50 mL) and extracted with EtOAc (50 mL×3). The combined organic layers were dried over Na₂SO₄ and concentrated under reduced pressure. The residue was purified by column chromatography (DCM:MeOH, 30:1, v/v) to afford the title compound (500 mg, 31%) as a yellow solid. LCMS: [M+H]⁺ 477.2.

### Step 6: 7-(Cyclopentylamino)-5-fluoro-2-((piperidin-4-ylthio)methyl)quinazolin-4(3H)-one Hydrochloride

A solution of tert-butyl 4-(((7-(cyclopentylamino)-5-fluoro-4-oxo-3,4-dihydroquinazolin-2-yl)methyl)thio)piperidine-1-carboxylate (450 mg, 0.94 mmol) in 40 mL of 1.5 M HCl in EtOAc was stirred at 25° C. overnight. The mixture was concentrated under reduced pressure to afford 7-(cyclopentylamino)-5-fluoro-2-(4-piperidylsulfanylmethyl)-3H-quinazolin-4-one (350 mg, 98.5% yield) as a yellow solid. LCMS: [M+H]⁺ 377.2.

¹H NMR (400 MHz, DMSO-d₆) δ 8.90 (br s, 1H), 8.77 (br s, 1H), 6.53 (s, 1H), 6.51 (d, J=14.0 Hz, 1H), 3.80-3.77 (m, 1H), 3.73 (s, 2H), 3.25-3.22 (m, 2H), 3.17-3.11 (m, 1H), 2.94-2.86 (m, 2H), 2.16-2.13 (m, 2H), 2.02-1.93 (m, 2H), 1.78-1.66 (m, 4H), 1.63-1.52 (m, 2H), 1.50-1.42 (m, 2H).

### Step 7: Tert-Butyl 2-[4-[[7-(cyclopentylamino)-5-fluoro-4-oxo-3H-quinazolin-2-yl]methylsulfanyl]-1-piperidyl]acetate

To a solution of 7-(cyclopentylamino)-5-fluoro-2-((piperidin-4-ylthio)methyl)quinazolin-4(3H)-one hydrochloride (1.0 g, 2.4 mmol) in NMP (10 mL) were added tert-butyl 2-bromoacetate (567 mg, 2.91 mmol) and K₂CO₃ (1.0 g, 7.26 mmol). The mixture was heated at 40° C. overnight. The mixture was allowed to cool to RT and filtered. The filtrate was diluted with water (30 mL) and extracted with EtOAc (30 mL×3). The combined organic layers were dried over Na₂SO₄ and concentrated under reduced pressure. The residue was purified by column chromatography (DCM:MeOH, 100:1 to 50:1, v/v) to afford the title compound (430 mg, 36%) as a yellow solid. LCMS: [M+H]⁺ 491.1.

### Step 8: 2-[4-[[7-(Cyclopentylamino)-5-fluoro-4-oxo-3H-quinazolin-2-yl]methylsulfanyl]-1-piperidyl]acetic acid Hydrochloride

A solution of tert-butyl 2-[4-[[7-(cyclopentylamino)-5-fluoro-4-oxo-3H-quinazolin-2-yl]methylsulfanyl]-1-piperidyl]acetate (430 mg, 0.88 mmol) in 2 M HCl/EtOAc (12 mL, 24 mmol) was stirred at 25° C. for 16 h. The reaction mixture was concentrated under reduced pressure to afford the title compound (390 mg, 94.5% yield) as a yellow solid. LCMS: [M+Na]+ 435.2.

### Step 9: 2-[4-[[7-(Cyclopentylamino)-5-fluoro-4-oxo-3H-quinazolin-2-yl]methylsulfanyl]-1-piperidyl]-N-[6-[[2-(2,6-dioxo-3-piperidyl)-1,3-dioxo-isoindolin-4-yl]amino]hexyl]acetamide Trifluoroacetate

To a solution of 2-[4-[[7-(cyclopentylamino)-5-fluoro-4-oxo-3H-quinazolin-2-yl]methylsulfanyl]-1-piperidyl]acetic acid hydrochloride (127 mg, 0.27 mmol) and 4-(6-aminohexylamino)-2-(2,6-dioxo-3-piperidyl)isoindoline-1,3-dione hydrochloride (110 mg, 0.27 mmol) in DMF (20 mL) under a N₂ atmosphere was added EDCI (258 mg, 1.35 mmol), HOBt (91 mg, 0.67 mmol) and DIPEA (139 mg, 1.08 mmol), and the mixture was stirred at RT overnight. The mixture was diluted with water (30 mL) and extracted with EtOAc (30 mL×3). The combined organic layers were dried over Na₂SO₄ and concentrated under reduced pressure. The residue was purified by column chromatography (petroleum ether:EtOAc, 1:1, v/v), reverse phase column (Biotage, 45-55% ACN in water, 0.1% TFA), and preparative HPLC (Shimadzu, Sepax BR prep-C18, 10 μm, 250×21.2 mm column, eluting with a gradient of ACN in water with 0.1% TFA, at a flow rate of 20 mL/min) to afford the title compound (85 mg, 35% yield) as a green solid. LCMS: [M+H]+ 789.2. ¹ HNMR (400 MHz, DMSO-d₆) δ 11.11 (s, 1H), 9.73 (s, 1H), 8.55-8.44 (m, 1H), 7.60-7.57 (m, 1H), 7.10-7.08 (m, 1H), 7.04-7.02 (m, 1H), 6.88 (s, 1H), 6.56-6.36 (m, 3H), 5.07-5.03 (m, 1H), 3.96-3.74 (m, 3H), 3.59 (s, 1H), 3.50-3.41 (m, 2H), 3.34-3.25 (m, 2H), 3.20-2.99 (m, 4H), 2.97-2.84 (m, 2H), 2.63-2.54 (m, 2H), 2.22-2.21 (m, 2H), 2.09-1.84 (m, 4H), 1.80-1.63 (m, 4H), 1.61-1.52 (m, 4H), 1.49-1.39 (m, 4H), 1.37-1.27 (m, 4H).

### Example 2: (2S,4R)-1-((S)-2-(7-(2-(4-(((7-(cyclopentylamino)-5-fluoro-4-oxo-3,4-dihydroquinazolin-2-yl)methyl)thio)piperidin-1-yl)acetamido)heptanamido)-3,3-dimethylbutanoyl)-4-hydroxy-N-(4-(4-methylthiazol-5-yl)benzyl)pyrrolidine-2-carboxamide

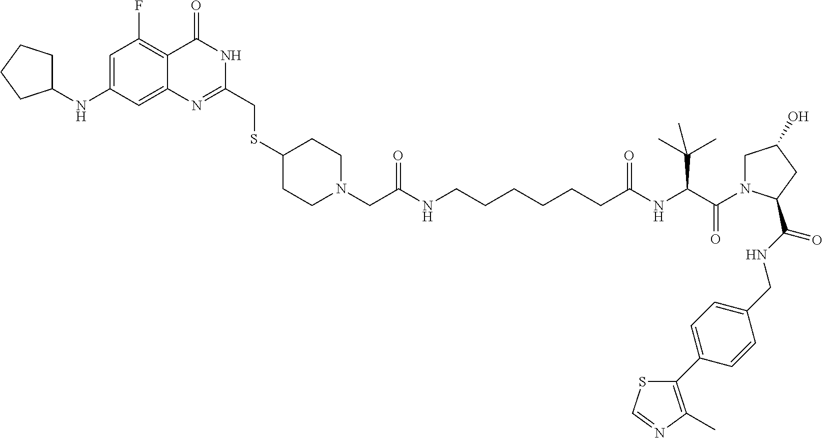 [SMILES: Cc1ncsc1-c1ccc(CNC(=O)[C@@H]2C[C@@H](O)CN2C(=O)[C@@H](NC(=O)CCCCCCNC(=O)CN2CCC(SCc3nc4cc(NC5CCCC5)cc(F)c4c(=O)[nH]3)CC2)C(C)(C)C)cc1]

### Step 1: Ethyl 7-[[2-[4-[[7-(cyclopentylamino)-5-fluoro-4-oxo-3H-quinazolin-2-yl]methylsulfanyl]-1-piperidyl]acetyl]amino]heptanoate

To a solution of 2-[4-[[7-(cyclopentylamino)-5-fluoro-4-oxo-3H-quinazolin-2-yl]methylsulfanyl]-1-piperidyl]acetic acid hydrochloride (200 mg, 0.42 mmol; from Example 1, Step 8) in DMF (10 mL) at RT under a N₂ atmosphere were added ethyl 7-aminoheptanoate (160 mg, 0.92 mmol), EDCI (265 mg, 1.38 mmol), triethylamine (233 mg, 2.3 mmol) and HOBt (187 mg, 1.38 mmol). The mixture was stirred at RT overnight. The mixture was diluted with water (20 mL), extracted with EtOAc (20 mL×3) and the combined organic layers were washed with water (20 mL), dried over Na₂SO₄ and concentrated under reduced pressure. The residue was purified by preparative TLC (DCM:MeOH, 10:1, v/v) to afford the title compound (170 mg, 61% yield) as a yellow solid. LCMS: [M+H]⁺ 590.2.

### Step 2: 7-[[2-[4-[[7-(Cyclopentylamino)-5-fluoro-4-oxo-3H-quinazolin-2-yl]methylsulfanyl]-1-piperidyl]acetyl]amino]heptanoic Acid

To a solution of ethyl 7-[[2-[4-[[7-(cyclopentylamino)-5-fluoro-4-oxo-3H-quinazolin-2-yl]methylsulfanyl]-1-piperidyl]acetyl]amino]heptanoate (168 mg, 0.28 mmol) in MeOH (5 mL/was added 2 N NaOH (0.57 mL, 1.14 mmol). The mixture was stirred at RT for 4 h. The mixture was concentrated to the title compound (128 mg, 0.23 mmol, 80% yield) as a yellow solid. LCMS: [M+H]⁺ 562.1.

### Step 3: (2S,4R)-1-((S)-2-(7-(2-(4-(((7-(cyclopentylamino)-5-fluoro-4-oxo-3,4-dihydroquinazolin-2-yl)methyl)thio)piperidin-1-yl)acetamido)heptanamido)-3,3-dimethylbutanoyl)-4-hydroxy-N-(4-(4-methylthiazol-5-yl)benzyl)pyrrolidine-2-carboxamide

To a solution of 7-[[2-[4-[[7-(cyclopentylamino)-5-fluoro-4-oxo-3H-quinazolin-2-yl]methylsulfanyl]-1-piperidyl]acetyl]amino]heptanoic acid (50 mg, 0.09 mmol) in NMP (5 mL) were added (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide (115 mg, 0.27 mmol), EDCI (42 mg, 0.27 mmol), HOBt (36 mg, 0.27 mmol), DIPEA (46 mg, 0.36 mmol). The mixture was stirred at RT overnight, then water was added and the resulting suspension was filtered. The filtrate was purified by preparative-HPLC (Shimadzu, Sepax BR prep-C18, 10 μm, 250×21.2 mm column, eluting with a gradient of ACN in water with 0.1% TFA, at a flow rate of 20 mL/min) to afford the title compound (5 mg, 6% yield) as a yellow solid. LCMS: [M+H]⁺ 975.2. ¹HNMR (400 MHz, CD₃OD) δ 8.98 (s, 1H), 7.48-7.41 (m, 5H), 6.47 (t, J=12.0 Hz, 2H), 4.65-4.62 (m, 1H), 4.58-4.51 (m, 3H), 4.38-4.34 (t, J=15.6 Hz, 1H), 3.91-3.78 (m, 5H), 3.70-3.60 (m, 2H), 3.25-3.21 (m, 2H), 3.13-3.07 (m, 2H), 2.48 (s, 3H), 2.30-2.19 (m, 5H), 2.11-2.00 (m, 4H), 1.87-1.83 (m, 2H), 1.79-1.75 (m, 2H), 1.69-1.64 (m, 2H), 1.62-1.50 (m, 6H), 1.34-1.29 (m, 6H), 1.03 (s, 9H).

### Example 3: 8-((2-(2,6-dioxopiperidin-3-yl)-1,3-dioxoisoindolin-4-yl)amino)-N-((1r,4r)-4-(((5-fluoro-4-oxo-7-((tetrahydro-2H-pyran-4-yl)methoxy)-3,4-dihydroquinazolin-2-yl)methyl)thio)cyclohexyl)octanamide

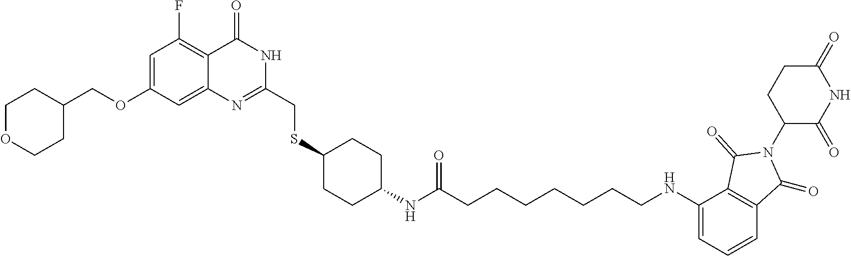 [SMILES: O=C1CCC(N2C(=O)c3cccc(NCCCCCCCC(=O)N[C@H]4CC[C@H](SCc5nc6cc(OCC7CCOCC7)cc(F)c6c(=O)[nH]5)CC4)c3C2=O)C(=O)N1]

### Step 1: 2,6-Difluoro-4-hydroxy-benzoic Acid

To a solution of 2,6-difluoro-4-hydroxy-benzonitrile (200 g, 1290 mmol) in water (933 mL) was added a solution of NaOH (181 g, 4513 mmol) in water (533 mL), and the mixture was stirred at 100° C. for 4 h. The mixture was cooled to room temperature and adjusted pH 2 with 6 N HCl. The suspension was filtered. The cake was washed with water (500 mL), and dried under vacuum to afford the title compound (222.2 g, 99% yield) as white solid. LCMS: [M−H] 173.0.

### Step 2: Methyl 2,6-difluoro-4-hydroxy-benzoate

To a solution of 2,6-difluoro-4-hydroxy-benzoic acid (811 g, 4658 mmol) in methanol (3500 mL) at 0° C. was added thionyl chloride (1386 g, 11646 mmol) slowly, and the mixture was refluxed overnight. The mixture was concentrated, and the residue was diluted with water (2500 mL) and stirred at room temperature for 30 min. The suspension was filtered. The cake was washed with water and dried under vacuum to afford the title compound (739 g, 84% yield) as an off-white solid. LCMS: [M+H]⁺ 189.1.

### Step 3: Methyl 2,6-difluoro-4-(tetrahydropyran-4-ylmethoxy)benzoate

A mixture of methyl 2,6-difluoro-4-hydroxy-benzoate (20 g, 106 mmol), 4-(bromomethyl)tetrahydropyran (22.8 g, 127.6 mmol) and K₂CO₃ (22 g, 160 mmol) in DMSO (150 mL) was stirred at 80° C. for 16 h under a nitrogen atmosphere. After cooling to room temperature, the reaction mixture was diluted with water (1000 mL). The precipitate was collected by filtration, and dried under vacuum to afford the title compound (30 g, 99% yield) as a yellow solid. LCMS: [M+H]⁺ 287.2.

### Step 4: Methyl 2-[(2,4-dimethoxyphenyl)methylamino]-6-fluoro-4-(tetrahydropyran-4-ylmethoxy)benzoate

A mixture of methyl 2,6-difluoro-4-(tetrahydropyran-4-ylmethoxy)benzoate (30 g, 105 mmol), (2,4-dimethoxyphenyl)methanamine (23.6 mL, 157.2 mmol) and K₂CO₃ (36.2 g, 262 mmol) in NMP (200 mL) was stirred at 80° C. for 16 h. After cooling to room temperature, the mixture was diluted with water (1500 mL) and extracted with EtOAc (300 mL×3). The combined organic layers were dried over Na₂SO₄ and concentrated under reduced pressure to afford the title compound (40 g, 88% yield) as a yellow solid. ¹H NMR (400 MHz, DMSO-d₆) δ 8.05-8.03 (m, 1H), 7.18-7.16 (m, 1H), 6.59 (s, 1H), 6.49-6.45 (m, 1H), 6.05-6.01 (m, 2H), 4.26 (d, 2H, J=5.6 Hz), 3.90-3.83 (m, 4H), 3.81 (s, 3H), 3.75 (s, 6H), 3.35-3.29 (m, 2H), 1.96-1.91 (m, 1H), 1.68-1.62 (m, 2H), 1.31-1.26 (m, 2H).

### Step 5: Methyl 2-amino-6-fluoro-4-(tetrahydropyran-4-ylmethoxy)benzoate

To a solution of methyl 2-[(2,4-dimethoxyphenyl)methylamino]-6-fluoro-4-(tetrahydropyran-4-ylmethoxy)benzoate (40 g, 92 mmol) and triethylsilane (29.5 mL, 184.6 mmol) in DCM (200 mL) was added TFA (100 mL, 1346 mmol). The reaction mixture was stirred at 25° C. for 2 h. The mixture was adjusted to pH 8-9 with a saturated aqueous NaHCO₃ solution and extracted with EtOAc (400 mL×3). The combined organic layers were dried over Na₂SO₄ and concentrated under reduced pressure to afford the title compound (20 g, 77% yield) as a yellow solid. LCMS: [M+H]⁺ 284.2.

### Step 6: 2-(Chloromethyl)-5-fluoro-7-((tetrahydro-2H-pyran-4-yl)methoxy)quinazolin-4(3H)-one

A mixture of methyl 2-amino-6-fluoro-4-(tetrahydropyran-4-ylmethoxy)benzoate (20 g, 71 mmol) and 2-chloroacetonitrile (13.4 mL, 211.8 mmol) in 2 N HCl/Dioxane (120 mL, 240 mmol) was stirred at 80° C. for 2 h under a nitrogen atmosphere. After cooling to room temperature, the precipitate was collected by filtration, washed with EtOAc (100 mL) and water (100 mL). The residue was dried under vacuum to afford the title compound (14 g, 61%) as a yellow solid. LCMS: [M+H]⁺ 327.1.

### Step 7: Tert-Butyl ((1r,4r)-4-(((5-fluoro-4-oxo-7-((tetrahydro-2H-pyran-4-yl)methoxy)-3,4-dihydroquinazolin-2-yl)methyl)thio)cyclohexyl)carbamate

To a solution of 2-(chloromethyl)-5-fluoro-7-(tetrahydropyran-4-ylmethoxy)-3H-quinazolin-4-one (200 mg, 0.61 mmol) and S-[4-(tert-butoxycarbonylamino)cyclohexyl]ethanethioate (201 mg, 0.73 mmol) in THF (2 mL) was added 2 N NaOH (2 mL, 4 mmol) and the mixture was stirred at RT overnight under a nitrogen atmosphere. The reaction was quenched with water (20 mL) and the mixture was extracted with EtOAc (20 mL×3). The combined organic layers were dried over Na₂SO₄ and concentrated under reduced pressure. The residue was purified by column chromatography (DCM:MeOH, 30:1, v/v) to afford the title compound (250 mg, 78%) as a brown solid. LCMS: [M+H]⁺ 522.3.

### Step 8: 2-[(4-aminocyclohexyl)sulfanylmethyl]-5-fluoro-7-(tetrahydropyran-4-ylmethoxy)-3H-quinazolin-4-one hydrochloride

A solution of tert-butyl ((1r,4r)-4-(((5-fluoro-4-oxo-7-((tetrahydro-2H-pyran-4-yl)methoxy)-3,4-dihydroquinazolin-2-yl)methyl)thio)cyclohexyl)carbamate (250 mg, 0.48 mmol) in 1 N HCl/EtOAc (5 mL, 5 mmol) was stirred at 25° C. for 2 h. The residue was concentrated under reduced pressure to afford the title compound (120 mg, 55%) as a brown solid. LCMS: [M+H]⁺ 422.3.

### Step 9: 8-[[2-(2,6-dioxo-3-piperidyl)-1,3-dioxo-isoindolin-4-yl]amino]-N-[4-[[5-fluoro-4-oxo-7-(tetrahydropyran-4-ylmethoxy)-3H-quinazolin-2-yl]methylsulfanyl]cyclohexyl]octanamide

To a solution of 8-[[2-(2,6-dioxo-3-piperidyl)-1,3-dioxo-isoindolin-4-yl]amino]octanoic acid (50 mg, 0.12 mmol) in DMF (1 mL) was added HATU (69 mg, 0.18 mmol), and the mixture was stirred at RT for 0.5 h. To the reaction mixture was added DIPEA (31 mg, 0.24 mmol) and 2-[(4-aminocyclohexyl)sulfanylmethyl]-5-fluoro-7-(tetrahydropyran-4-ylmethoxy)-3H-quinazolin-4-one hydrochloride (66 mg, 0.14 mmol), and the mixture was stirred at RT for 2 h. The mixture was diluted with water (10 mL) and extracted with EtOAc (15 mL×3). The combined organic layers were dried over Na₂SO₄ and concentrated under reduced pressure. The residue was purified by preparative-TLC (DCM:MeOH, 20:1, v/v) to afford the title compound (45 mg, 46%) as a yellow solid. LCMS: [M+H]⁺ 819.0. ¹H NMR (400 MHz, DMSO-d₆) δ 12.16 (s, 1H), 11.10 (s, 1H), 7.64-7.53 (m, 2H), 7.10-7.06 (m, 1H), 7.03-6.99 (m, 1H), 6.91-6.85 (m, 2H), 6.59-6.49 (m, 1H), 5.10-4.99 (m, 1H), 4.01-3.93 (m, 2H), 3.92-3.84 (m, 2H), 3.59 (s, 2H), 3.53-3.44 (m, 1H), 3.31-3.27 (m, 2H), 2.94-2.81 (m, 1H), 2.73-2.64 (m, 1H), 2.62-2.54 (m, 1H), 2.05-1.95 (m, 6H), 1.90-1.72 (m, 2H), 1.70-1.61 (m, 2H), 1.59-1.50 (m, 2H), 1.49-1.41 (m, 2H), 1.37-1.09 (m, 15H).

### Example 4: 3-((2-(2,6-dioxopiperidin-3-yl)-1,3-dioxoisoindolin-4-yl)amino)-N-((1r,4r)-4-(((5-fluoro-4-oxo-7-((tetrahydro-2H-pyran-4-yl)methoxy)-3,4-dihydroquinazolin-2-yl)methyl)thio)cyclohexyl)propanamide

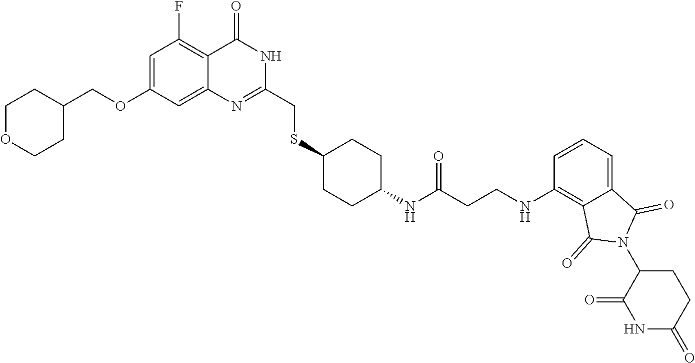 [SMILES: O=C1CCC(N2C(=O)c3cccc(NCCC(=O)N[C@H]4CC[C@H](SCc5nc6cc(OCC7CCOCC7)cc(F)c6c(=O)[nH]5)CC4)c3C2=O)C(=O)N1]

To a solution of 2-[(4-aminocyclohexyl)sulfanylmethyl]-5-fluoro-7-(tetrahydropyran-4-ylmethoxy)-3H-quinazolin-4-one hydrochloride (30 mg, 0.07 mmol; from Example 3, Step 8) and EDCI (25 mg, 0.13 mmol) in DMF (1 mL) were added HOBt (18 mg, 0.13 mmol), triethylamine (27 mg, 0.26 mmol) and 3-[[2-(2,6-dioxo-3-piperidyl)-1,3-dioxo-isoindolin-4-yl]amino]propanoic acid (27 mg, 0.08 mmol). The mixture was stirred at RT for 3 h. Water (2 mL) was added to the mixture. The resulting precipitate was collected by filtration, washed with water and dried under vacuum. The crude material was purified by reverse phase column (H₂O/ACN=60/40 v/v) to give the title compound (15 mg, 31%) as a yellow solid. LCMS: [M+H]⁺ 748.9; ¹HNMR (400 MHz, DMSO-d₆) δ 12.18 (s, 1H), 11.10 (s, 1H), 7.08 (d, J=7.6 Hz, 1H), 7.60-7.56 (m, 1H), 7.12 (d, J=8.8 Hz, 1H), 7.02 (d, J=6.8 Hz, 1H), 6.91-6.87 (m, 2H), 6.71 (s, 1H), 5.07-5.01 (m, 1H), 3.98 (d, J=6.4 Hz, 2H), 3.88-3.86 (m, 2H), 3.59 (s, 2H), 3.55-3.45 (m, 3H), 3.36-3.27 (m, 2H), 2.90-2.84 (m, 1H), 2.74-2.64 (m, 1H), 2.60-2.56 (m, 1H), 2.45-2.42 (m, 1H), 2.39-2.31 (m, 2H), 2.05-1.94 (m, 4H), 1.80-1.76 (m, 2H), 1.68-1.65 (m, 2H), 1.38-1.23 (m, 4H), 1.20-1.08 (m, 2H).

### Example A. Enzymatic Assay for Inhibition of PARP14

The catalytic domain of human PARP14 (residues 1611 to 1801, GenBank Accession No. NM_017554) was overexpressed in Escherichia coli cells. An N-terminal His-TEV fusion tag was used to purify the protein from cell lysates. The His-TEV tag was left on the protein for use in the enzymatic assay.

Enzymatic inhibition of PARP14 was measured using a dissociation-enhanced lanthanide fluorescence immunoassay (DELFIA) monitoring the auto-modification of PARP14 by biotinylated nicotinamide adenine dinucleotide (biotin-NAD). 1 μL of a dose response curve of each test compound was spotted in 384-well nickel-coated white microplates (Thermo) using a Mosquito (TTP Labtech). Reactions were performed in a 50 μL volume by adding 40 μL of PARP14 in assay buffer (20 mM HEPES pH=8, 100 mM NaCl. 0.1% bovine serum albumin, 2 mM DTT and 0.002% Tween20), incubating with test compound at 25° C. for 30 min, then adding 10 μL of biotin-NAD (Biolog). The final concentrations of PARP14 and biotin-NAD are 50 nM and 3 μM, respectively. Reactions proceeded at 25° C. for 3 h, then were quenched with 5 μL of 10 mM unmodified nicotinamide adenine dinucleotide (Sigma-Aldrich). The quenched reactions were washed 3 times with 100 μL of TBST wash buffer (50 mM Tris-HCl, 150 mM NaCl and 0.1% Tween20). Next, to the washed and dried plate was added 25 μL of DELFIA Europium-N1 streptavidin (Perkin Elmer) diluted in DELFIA assay buffer (Perkin Elmer). After a 30 min incubation at 25° C., the plate was washed 5 times with TBST wash buffer. Finally. 25 μL of DELFIA enhancement solution was added. After a 5 min incubation the plate was read on an Envision platereader equipped with a LANCE/DELFIA top mirror (Perkin Elmer) using excitation of 340 nm and emission of 615 nm to measure the amount of Europium present in each well, informing on the amount of biotin-NAD that was transferred in the automodification reaction. Control wells containing a negative control of 2% DMSO vehicle or a positive control of 100 μM rucaparlb were used to calculate the % inhibition as described below:

$$\% inhibition = 100 \times \frac{ex {615}_{cmpd} - ex {615}_{min}}{ex {615}_{max} - ex {615}_{min}}$$

where ex615_{cmpd} is the emission from the compound treated well, ex615ₘᵢₙ is the emission from the rucaparib treated positive control well and ex615ₘₐₓ is the emission from the DMSO treated negative control well.

The % inhibition values were plotted as a function of compound concentration and the following 4-parameter fit was applied to derive the IC₅₀ values:

$$Y = Bottom + \frac{( Top - Bottom )}{( 1 + {( \frac{X}{{IC}_{50}} )}^{Hill Coefficient}}$$

where top and bottom are normally allowed to float, but may be fixed at 100 or 0 respectively in a 3-parameter fit. The Hill Coefficient is normally allowed to float but may also be fixed at 1 in a 3-parameter fit. Y is the % inhibition and X is the compound concentration.

IC₅₀ data for certain compounds corresponding to group Q as defined herein, is provided below in Table A-1 (“+” is <1 μM; “++” is ≥1 μm and <10 μM; and “+++” is ≥10 μM).

TABLE A-1

| | IC₅₀ PARP14 |
|---|---|
| Compound | (μM) |
| 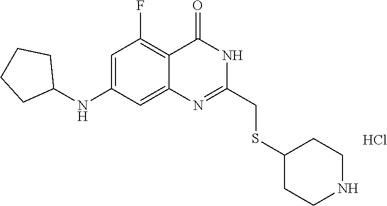 [SMILES: O=c1[nH]c(CSC2CCNCC2)nc2cc(NC3CCCC3)cc(F)c12 ; Cl] | + |
| 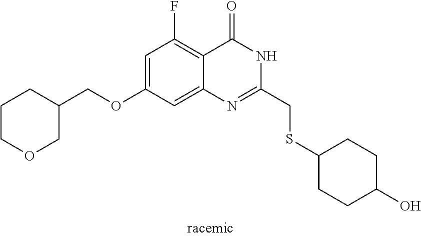 [SMILES: O=c1[nH]c(CSC2CCC(O)CC2)nc2cc(OCC3CCCOC3)cc(F)c12] | + |
| 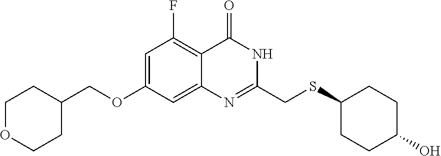 [SMILES: O=c1[nH]c(CS[C@H]2CC[C@H](O)CC2)nc2cc(OCC3CCOCC3)cc(F)c12] | + |
| 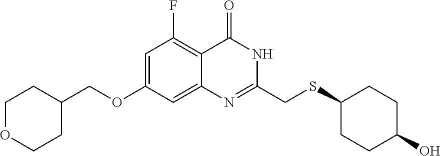 [SMILES: O=c1[nH]c(CS[C@H]2CC[C@@H](O)CC2)nc2cc(OCC3CCOCC3)cc(F)c12] | + |

### Example B: PARP14 Degradation Assay

KYSE270 cells were seeded at a density of 0.5e⁶ cells/well in 6-well plates and incubated overnight. Once attached, cells were treated with the compounds of Examples 1-4 at increasing concentrations (0.001 μM, 0.01 μM, 0.1 μM, 1 μM, and 10 μM; 0.003 μM, 0.03 μM, 0.3 μM, and 3 μM were also evaluated for the compound of Example 1), or with DMSO for 24 h. Media was gently aspirated and cells washed 3 times with 2 mL of ice cold PBS while on ice. The PBS was completely aspirated and 75 μl freshly prepared lysis buffer (Thermo Fisher 78501) was added to cells before scraping into the buffer. Lysates were collected in microcentrifuge tubes and incubated on ice for 15 minutes. Lysates were centrifuged at 10,000 rpm for 15 min at 4° C. and supernatants collected into fresh microcentrifuge tubes. Protein concentration was measured using a reducing agent compatible with the Pierce BCA Protein Assay Kit (Thermo Fisher 23250). Samples were prepared in loading buffer (LI-COR 928-40004) containing 5% β-mercaptoethanol, and incubated at 95° C. for 5 min. Protein lysates were resolved on 4-12% Tris-Acetate gels in MOPS running buffer with 60 μg of protein per well. Western blot transfers were done with PVDF membranes (LI-COR Immobilon) with 20 volts for 14 minutes. Primary antibodies (PARP14: in house generated mouse antibody (15A6 Lot1C), β-actin: D6A8 (8457)) were incubated at 1:1,000 dilution using the odyssey blocking buffer (LI-COR 927-50000) for 2 h at room temperature and detected with secondary antibody (LI-COR 926-68072, 926-32211). Mouse antibody 15A6 Lot1C was generated by immunizing with recombinant human PARP14 catalytic domain protein. After hybridoma fusion, the parental clones were screened for reactivity against PARP14 catalytic domain, which were then subcloned to generate monoclones including 15A6-1. Monoclonal supernatants were tested by Western blotting against THP-1 and THP-1 PARP14 KO cells to confirm reactivity. The PARP14 antibody was produced by culturing the 15A6-1 hybridoma monoclone in 1 L of serum free media+2% low IgG FBS. The antibody was purified from the culture media by protein G affinity chromatography.

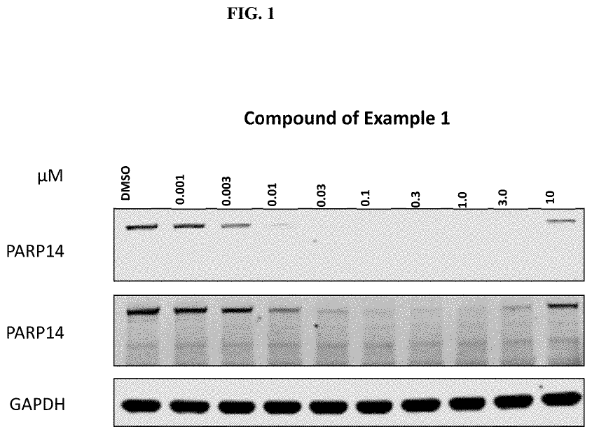

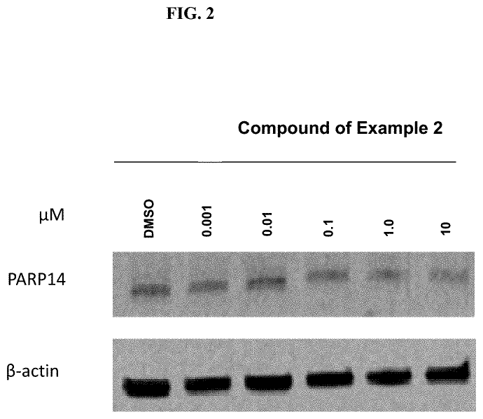

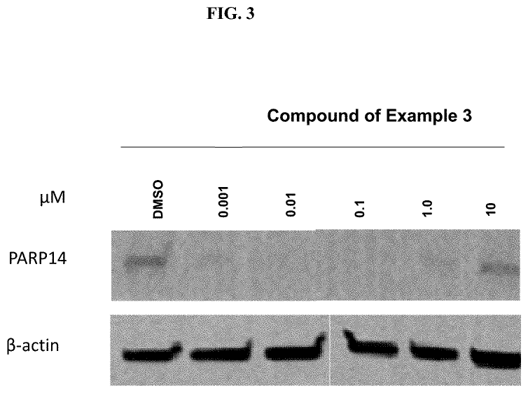

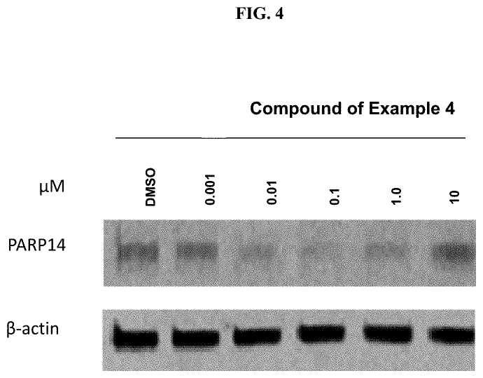

Treatment of KYSE270 cells with the compounds of Examples 1-4 for 24 h results in dose dependent depletion of PARP14. At higher concentrations, Examples 1, 3, and 4 demonstrate amelioration of efficacy consistent with a ternary complex-mediate mechanism, which is known as “the hook effect” and is described in Crews et al, Nature Chem. Biol. 2015, 11, 611. FIG. 1 shows the Western blot of the PARP14 degradation assay for the compound of Example 1. FIG. 2 shows the Western blot of the PARP14 degradation assay for the compound of Example 2. FIG. 3 shows the Western blot of the PARP14 degradation assay for the compound of Example 3. FIG. 4 shows the Western blot of the PARP14 degradation assay for the compound of Example 4.

### Example C: mRNA Expression Levels of PARP14 in Various Cancer Types

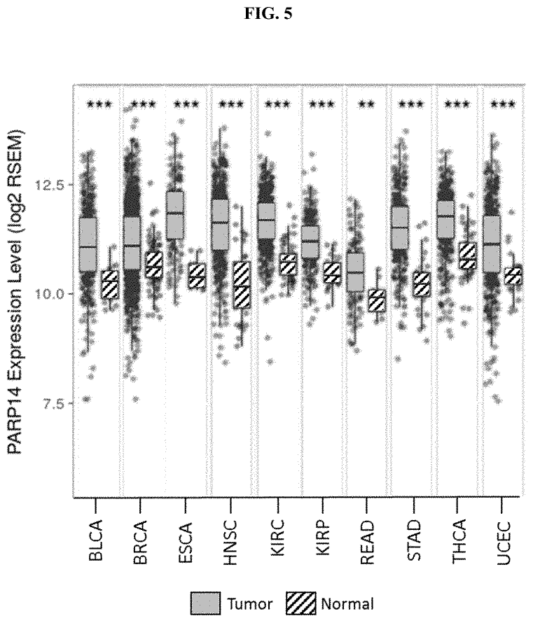

FIG. 5 illustrates the mRNA expression levels of PARP14 in various cancer types, compared to their matched normal tissue. RNA sequencing data were downloaded from The Cancer Genome Consortium (TCGA) and analyzed. Individual dots represent values from individual samples, boxes represent the interquartile or middle 50% of the data with horizontal lines being the group median, vertical lines representing the upper and lower quartiles of the data. It is apparent that PARP14 mRNA is higher, compared to normal tissue, in several cancer types. BLCA=bladder cancer, BRCA=breast cancer, ESCA=esophageal cancer, HNSC=head and neck cancer, KIRP=papillary kidney cancer, KIRC=clear cell kidney cancer, READ=rectal cancer, STAD=stomach cancer, THCA=thyroid cancer, UCEC—uterine cancer. * p<0.05, ** p<0.01, *** p<0.001, Wilcoxon test.

### Example D: Reduction of IL-10 Production in Cells

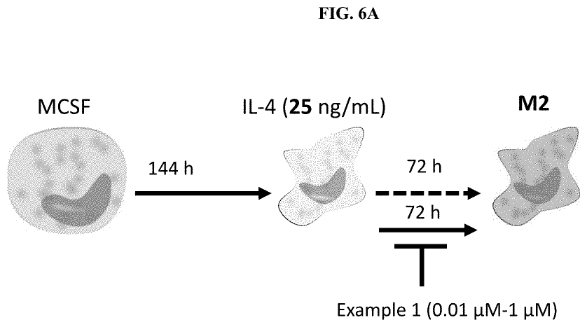

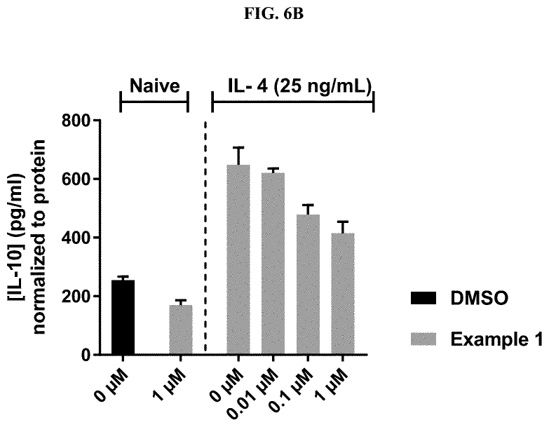

FIGS. 6A and 6B illustrate that in vitro treatment with the compound of Example 1 decreased IL-10 production in IL-4 stimulated M2-like macrophages. FIG. 6A shows the experimental layout.

Monocytes were isolated from peripheral human blood and cultured in the presence of M-CSF and the compound of Example 1 (at 1, 0.1 or 0.01 μM) for 72 h. M-CSF differentiates monocytes into M-0 macrophages. Subsequently medium was replaced with fresh medium containing IL-4 and the compound of Example 1 (at 1, 0.1 or 0.01 μM), and cells were incubated for another 48 h.

FIG. 6B shows IL-10 levels in tissue culture supernatant, measured by ELISA, of cells treated as described above.

Isolation of primary human monocytes from whole blood: Primary monocytes were isolated from whole blood (iSPECIMEN; 500 mL) collected from healthy donors. Blood was diluted at a 1:1 ratio with EasySep buffer (STEMCELL Technologies 20144) and layered onto lymphoprep (STEMCELL Technologies 07811) in SepMate tubes (STEMCELL Technologies 85450) for PBMC isolation according to the manufacturer's instructions. The isolated PBMCs were pooled, washed with EasySep buffer, resuspended in the appropriate volume of ammonium chloride solution (STEMCELL Technologies 07850; 10-15 mL) for RBC lysis, and gently shaken for 10 minutes. The total volume was increased to 40 mL with EasySep buffer to dilute the RBC lysis, then cells were centrifuged at 1500 rpm for 5 minutes. Fresh EasySep buffer was used to resuspend PBMCs for counting. The EasySep human monocyte isolation kit (STEMCELL Technologies 19359) was used to isolate monocytes from the PBMC cell population according to the manufacturer's instructions. The enriched monocyte cell population was resuspended in fresh EasySep buffer for counting and seeding for subsequent assays.

Monocyte to macrophage differentiation, M2 polarization, and PARP14 inhibition: Monocytes were seeded on day 0 in ImmunoCult SF macrophage medium (STEMCELL Technologies 10961) containing 50 ng/mL M-CSF (STEMCELL Technologies 78057) at a density of 1 million cells per 1 mL of media in 12-well plates and allowed to grow and differentiate into macrophages for 6 days. On day 4, one half of the initial volume of media was added to each well. Six days after monocyte seeding, cells were treated with 25 ng/mL human recombinant IL-4 (STEMCELL Technologies 78045) and samples were collected (media and cells) at 72 hours. Cells were treated with the compound of Example 1 or DMSO on day 6 after seeding at 1 μmol/L, 0.1 μmol/L, and 0.01 μmol/L.

IL-10 determination: Levels of IL-10 in the supernatants of human primary M2 macrophages were determined with the IL-10 ELISA kit (STEMCELL Technologies 02013) according to the manufacturer's instructions. Briefly, supernatants were collected at the indicated time point and depleted of any floating cells before being stored at −80° C. until ready to use. IL-10 concentrations were determined from the kit's IL-10 standard curve and normalized to total cell protein.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A compound of Formula (A1):
Q-L¹-E (A1)
or a pharmaceutically acceptable salt thereof, wherein:
Q is a moiety represented by Formula I: 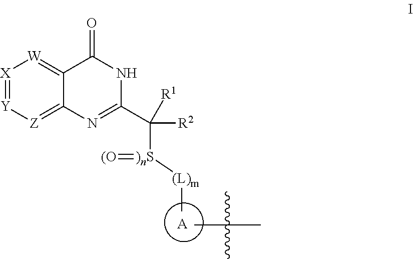
wherein:
W is CR^{W} or N;
X is CR^{X} or N;
Y is CR^{Y} or N;
Z is CR^{Z} or N;
wherein no more than two of W, X, Y, and Z are simultaneously N;
Ring A is monocyclic or polycyclic C₃₋₁₄ cycloalkyl or Ring A is monocyclic or polycyclic 4-18 membered heterocycloalkyl, wherein Ring A is optionally substituted by 1, 2, 3, or 4 R^{A}, and Ring A is attached to the -(L)ₘ- moiety of Formula I through a non-aromatic ring when Ring A is polycyclic;
L is —(CR⁵R⁶)ₜ—, —(CR⁵R⁶)ₚ—O—(CR⁵R⁶)_{q}—, —(CR⁵R⁶)ₚ—S—(CR⁵R⁶)_{q}—, —(CR⁵R⁶)ₚ—NR³—(CR⁵R⁶)_{q}—, —(CR⁵R⁶)ₚ—CO—(CR⁵R⁶)_{q}—, —(CR⁵R⁶)ᵣ—C(O)O—(CR⁵R⁶)ₛ—, —(CR⁵R⁶)ᵣ—CONR³—(CR⁵R⁶)ₛ—, —(CR⁵R⁶)ₚ—SO—(CR⁵R⁶)_{q}—, —(CR⁵R⁶)ₚ—SO₂—(CR⁵R⁶)_{q}—, —(CR⁵R⁶)ᵣ—SONR³—(CR⁵R⁶)ₛ—, or —NR³CONR⁴—;
R¹ and R² are each, independently, selected from H and methyl;
R³ and R⁴ are each, independently, selected from H and C₁₋₄ alkyl;
R⁵ and R⁶ are each, independently, selected from H, halo, C₁₋₄ alkyl, C₁₋₄ alkoxy, C₁₋₄ haloalkyl, amino, C₁₋₄ alkylamino, and C₂₋₈ dialkylamino;
each R^{A} is independently selected from halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{A} are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂-6 alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
R^{W}, R^{X}, R^{Y}, and R^{Z} are each, independently, selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{W}, R^{X}, R^{Y}, or R^{Z} are each optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
wherein when W is CR^{W}, X is CR^{X}, Y is CR^{Y}, and Z is CR^{Z}, then at least one of R^{W}, R^{X}, R^{Y}, and R^{Z} is other than H;
each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each Cy² is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
each R^{a1}, R^{b1}, R^{c1}, R^{d1}, R^{a2}, R^{b2}, R^{c2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a1}, R^{b1}, R^{c1}, R^{d1}, R^{a2}, R^{b2}, R^{c2}, or R^{d2} is optionally substituted with 1, 2, 3, 4, or 5 substituents independently selected from Cy³, Cy³-C₁₋₄ alkyl, halo, C₁₋₄ alkyl, C₁₋₄haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
each Cy³ is C₆₋₁₀ aryl, C₃₋₇ cycloalkyl, 5-10 membered heteroaryl, or 4-10 membered heterocycloalkyl, each optionally substituted by 1, 2, 3, or 4 substituents independently selected from halo, C₁₋₄ alkyl, C₁₋₄ haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
R^{a3}, R^{b3}, R^{c3}, and R^{d3} are independently selected from H, C₁₋₆ alkyl, C₁₋₆ haloalkyl, C₂-6 alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
or R^{c1} and R^{d1} together with the N atom to which they are attached form a 4-7 membered heterocycloalkyl group optionally substituted with 1, 2, or 3 substituents independently selected from halo, C₁₋₄ alkyl, C₁₋₄haloalkyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
or R^{c2} and R^{d2} together with the N atom to which they are attached form a 4-7 membered heterocycloalkyl group optionally substituted with 1, 2, or 3 substituents independently selected from halo, C₁₋₄ alkyl, C₁₋₄haloalkyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3} NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
each R^{e1}, R^{e2}, and R^{e3} is independently selected from H, C₁₋₄ alkyl, and CN;
m is 0 or 1,
n is 0, 1, or 2;
p is 0, 1, or 2;
q is 0, 1, or 2, wherein p+q is 0, 1, or 2;
r is 0 or 1;
s is 0 or 1, where r+s is 0 or 1; and
t is 1, 2, or 3;
L¹ is a linker, which is covalently linked to moiety Q and to moiety E;
E is an E3 ubiquitin ligase binding moiety, which binds to the E3 ubiquitin ligase; and
wherein the wavy lines represent the points of attachment to group L¹;
wherein any aforementioned heteroaryl or heterocycloalkyl group comprises 1, 2, 3, or 4 ring-forming heteroatoms independently selected from O, N, and S;
wherein one or more ring-forming C or N atoms of any aforementioned heterocycloalkyl group is optionally substituted by an oxo (═O) group; and
wherein one or more ring-forming S atoms of any aforementioned heterocycloalkyl group is optionally substituted by one or two oxo (═O) groups.

2. The compound of claim 1, or a pharmaceutically acceptable salt thereof, wherein linker L¹ is a chain of 1 to 40, 1 to 30, 1 to 25, 1 to 20, 1 to 15, 1 to 10, or 1 to 5 chain atoms, which is optionally substituted with 1-3 R^{q} substituents, and wherein one or more chain carbon atoms of L¹ can be oxidized to form a carbonyl (C═O), and wherein one or more N and S chain atoms can each be optionally oxidized to form an amine oxide, sulfoxide or sulfonyl group; and
each R^{q} is independently selected from OH, CN, —COOH, NH₂, halo, C₁₋₆ haloalkyl, C₁₋₆ alkyl, C₁₋₆ alkoxy, C₁₋₆haloalkoxy, C₁₋₆ alkylthio, phenyl, 5-6 membered heteroaryl, 4-6 membered heterocycloalkyl, C₃₋₆ cycloalkyl, NH(C₁₋₆ alkyl) and N(C₁₋₆ alkyl)₂, wherein the C₁₋₆ alkyl, phenyl, C₃₋₆ cycloalkyl, 4-6 membered heterocycloalkyl, and 5-6 membered heteroaryl of R^{q} are each optionally substituted with halo, OH, CN, —COOH, NH₂, C₁₋₄ alkyl, C₁₋₄ alkoxy, C₁₋₄ haloalkyl, C₁₋₄ haloalkoxy, phenyl, C₃₋₁₀ cycloalkyl, 5- or 6-membered heteroaryl or 4-6 membered heterocycloalkyl.

3. The compound of claim 1, or a pharmaceutically acceptable salt thereof, wherein linker L¹ has the structure: 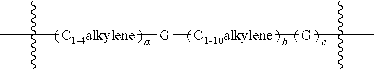
wherein each G is independently selected from —C(O)—, —NR^{G}C(O)—, —NR^{G}—, —O—, —S—, C(O)O—, —OC(O)NR^{G}—, —NR^{G}C(O)NR^{G}—, —S(O₂)—, or —S(O)NR^{G}—;
each R^{G} is independently selected from H, methyl, and ethyl;
a is 0 or 1;
b is 0 or 1; and
c is 0 or 1, wherein the wavy lines represent points of attachment to moieties Q and E.

4. The compound of claim 3, or a pharmaceutically acceptable salt thereof, wherein a is 1, b is 1, and c is 1.

5. The compound of claim 3, or a pharmaceutically acceptable salt thereof, wherein a is 0, b is 1, and c is 0.

6. The compound of claim 3, or a pharmaceutically acceptable salt thereof, wherein a is 1, b is 1, and c is 0.

7. The compound of claim 3, or a pharmaceutically acceptable salt thereof, wherein each G is independently selected from —C(O)— and —NR^{G}C(O)—.

8. The compound of claim 1, or a pharmaceutically acceptable salt thereof, wherein linker L¹ is selected from: 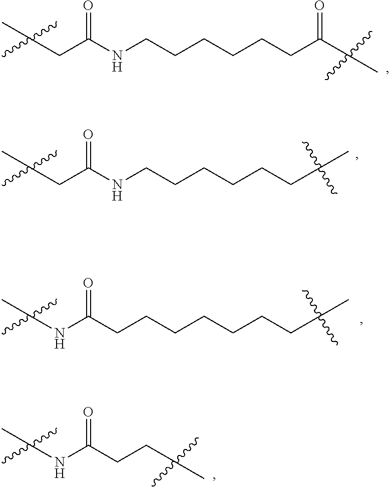 [SMILES: *CC(=O)NCCCCCCC(*)=O ; *CCCCCCNC(=O)C* ; *CCCCCCCC(=O)N* ; *CCC(=O)N*]
and
wherein the wavy lines represent points of attachment to moieties Q and E.

9. The compound of claim 1, or a pharmaceutically acceptable salt thereof, wherein E is a Von Hippel-Lindau (VHL) E3 ubiquitin ligase binding moiety.

10. The compound of claim 1, or a pharmaceutically acceptable salt thereof, wherein E is a moiety having a structure selected from: 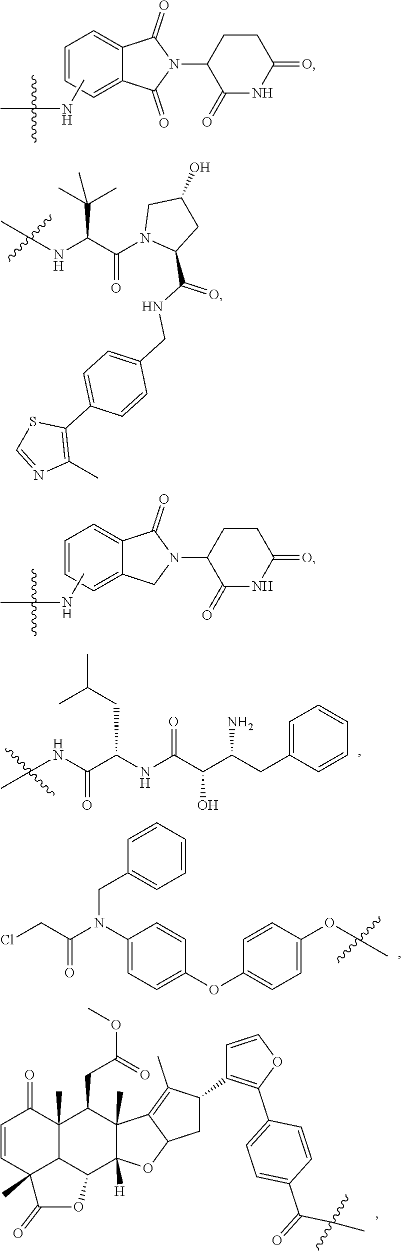 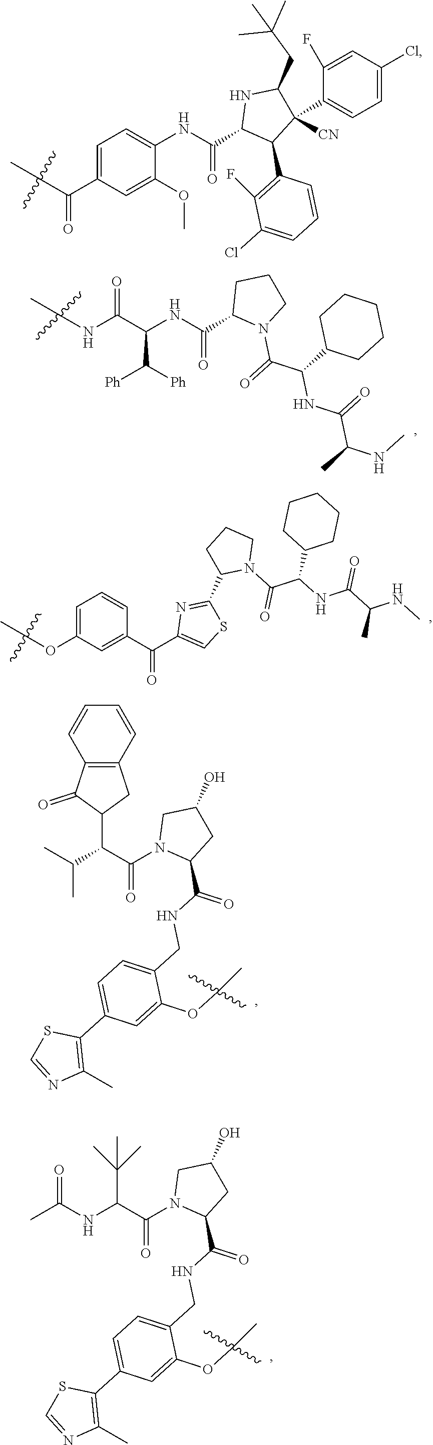 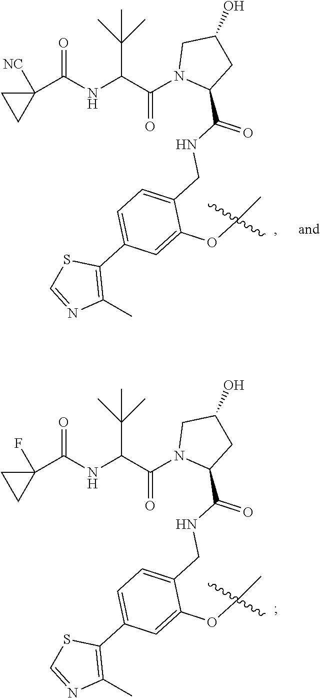 [SMILES: *Oc1cc(-c2scnc2C)ccc1CNC(=O)[C@@H]1C[C@@H](O)CN1C(=O)C(NC(=O)C1(C#N)CC1)C(C)(C)C ; *Oc1cc(-c2scnc2C)ccc1CNC(=O)[C@@H]1C[C@@H](O)CN1C(=O)C(NC(=O)C1(F)CC1)C(C)(C)C]
wherein the wavy lines represent the point of attachment to group L¹.

11. The compound of claim 1, or a pharmaceutically acceptable salt thereof, wherein E has the following structure: 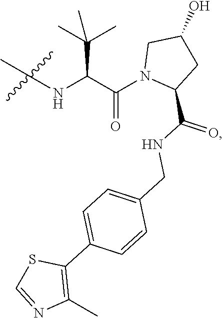 [SMILES: *N[C@H](C(=O)N1C[C@H](O)C[C@H]1C(=O)NCc1ccc(-c2scnc2C)cc1)C(C)(C)C]
wherein the wavy line represents the point of attachment to L¹.

12. The compound of claim 1, or a pharmaceutically acceptable salt thereof, wherein E has the following structure: 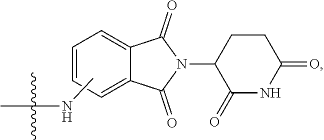
wherein the wavy line represents the point of attachment to L¹.

13. The compound of claim 1, or a pharmaceutically acceptable salt thereof, wherein E has the following structure: 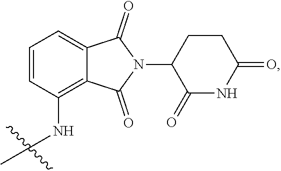 [SMILES: *Nc1cccc2c1C(=O)N(C1CCC(=O)NC1=O)C2=O]
wherein the wavy line represents the point of attachment to L¹.

14. The compound of claim 1, or a pharmaceutically acceptable salt thereof, wherein W is CR^{W}; X is CR^{X}; Y is CR^{Y}; and Z is CR^{Z}.

15. The compound of claim 1, or a pharmaceutically acceptable salt thereof wherein W is N; X is CR^{X}; Y is CR^{Y}; and Z is CR^{Z}.

16. The compound of claim 1, or a pharmaceutically acceptable salt thereof, wherein W is CR^{W}; X is N; Y is CR^{Y}; and Z is CR^{Z}.

17. The compound of claim 1, or a pharmaceutically acceptable salt thereof, wherein W is CR^{W}; X is CR^{X}; Y is N; and Z is CR^{Z}.

18. The compound of claim 1, or a pharmaceutically acceptable salt thereof, wherein W is CR^{W}; X is CR^{X}; Y is CR^{Y}; and Z is N.

19. The compound of claim 1, or a pharmaceutically acceptable salt thereof, wherein Ring A is monocyclic or polycyclic C₃₋₁₄ cycloalkyl optionally substituted by 1, 2, 3, or 4 R^{A}, wherein Ring A is attached to the -(L)ₘ-moiety of Formula I through a non-aromatic ring when Ring A is polycyclic.

20. The compound of claim 1, or a pharmaceutically acceptable salt thereof, wherein Ring A is cyclohexyl optionally substituted by 1, 2, 3, or 4 R^{A}.

21. The compound of claim 1, or a pharmaceutically acceptable salt thereof, wherein Ring A is monocyclic or polycyclic 4-18 membered heterocycloalkyl optionally substituted by 1, 2, 3, or 4 R^{A}, and wherein Ring A is attached to the -(L)ₘ- moiety of Formula I through a non-aromatic ring when Ring A is polycyclic.

22. The compound of claim 1, or a pharmaceutically acceptable salt thereof, wherein Ring A is piperidinyl optionally substituted by 1, 2, 3, or 4 R^{A}.

23. The compound of claim 1, or a pharmaceutically acceptable salt thereof, wherein Ring A is piperidin-4-yl optionally substituted by 1, 2, 3, or 4 R^{A}.

24. The compound of claim 1, or a pharmaceutically acceptable salt thereof, wherein L is —CH₂—.

25. The compound of claim 1, or a pharmaceutically acceptable salt thereof, wherein m is 0.

26. The compound of claim 1, or a pharmaceutically acceptable salt thereof, wherein m is 1.

27. The compound of claim 1, or a pharmaceutically acceptable salt thereof, wherein n is 0.

28. The compound of claim 1, or a pharmaceutically acceptable salt thereof, wherein R¹ and R² are both H.

29. The compound of claim 1, or a pharmaceutically acceptable salt thereof, wherein each R^{A} is independently selected from C₁₋₆ alkyl, OR^{a1}, C(O)R^{b1}, NR^{c1}R^{d1}, and S(O)₂R^{b1}; wherein said C₁₋₆ alkyl is optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

30. The compound of claim 1, or a pharmaceutically acceptable salt thereof, wherein each R^{A} is independently selected from halo, C₁₋₆ haloalkyl, OR^{a1}, C(O)NR^{c1}R^{d1}, and C(O)OR^{a1}.

31. The compound of claim 1, or a pharmaceutically acceptable salt thereof, wherein each R^{W}, R^{X}, R^{Y}, and R^{Z} is independently selected from H, halo, C₁₋₆ alkyl, C₁₋₆ haloalkyl, 5-10 membered heteroaryl, 4-10 membered heterocycloalkyl, C₆₋₁₀ aryl-C₁₋₄ alkyl, CN, OR^{a2}, C(O)NR^{c2}R^{d2}, NR^{c2}R^{d2}, and NR^{c2}C(O)R^{b2}; wherein said C₁₋₆ alkyl, C₁₋₆ haloalkyl, 5-10 membered heteroaryl, 4-10 membered heterocycloalkyl, and C₆₋₁₀ aryl-C₁₋₄ alkyl of R^{W}, R^{X}, R^{Y}, and R^{Z} are each optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}.

32. The compound of claim 1, or a pharmaceutically acceptable salt thereof, wherein W is CR^{W} and R^{W} is other than H.

33. The compound of claim 1, or a pharmaceutically acceptable salt thereof, wherein R^{W} is halo.

34. The compound of claim 1, or a pharmaceutically acceptable salt thereof, wherein R^{W} is F.

35. The compound of claim 1, or a pharmaceutically acceptable salt thereof, wherein X is CR^{X} and R^{X} is H.

36. The compound of claim 1, or a pharmaceutically acceptable salt thereof, wherein Y is CR^{Y} and R^{Y} is other than H.

37. The compound of claim 1, or a pharmaceutically acceptable salt thereof, wherein Y is CR^{Y} and R^{Y} is independently selected from C₁₋₆ alkyl, OR^{a2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, and NR^{c2}S(O)₂NR^{c2}R^{d2}.

38. The compound of claim 1, or a pharmaceutically acceptable salt thereof, wherein Y is CR^{Y} and R^{Y} is independently selected from C₁₋₆ alkyl, C₃₋₇ cycloalkyl-C₁₋₄ alkyl, 5-10 membered heteroaryl, 4-10 membered heterocycloalkyl, halo, CN, OR^{a2}, SR^{a2}, C(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2} NR^{c2}S(O)R^{b2}, NR^{c2}S(O)₂R^{b2}, and NR^{c2}S(O)₂NR^{c2}R^{d2}, wherein said C₁₋₆ alkyl, C₃₋₇ cycloalkyl-C₁₋₄ alkyl, 5-10 membered heteroaryl, and 4-10 membered heterocycloalkyl of R are each optionally substituted with 1, 2, 3, 4, or 5 substituents independently selected from halo, C₁₋₆ alkyl, C₁₋₆ haloalkyl, CN, NO₂, OR^{a2}, NR^{c2}R^{d2}, and S(O)₂R^{b2}.

39. The compound of claim 1, or a pharmaceutically acceptable salt thereof, wherein Y is CR^{Y} and R^{Y} is independently selected from NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, and NR^{c2}S(O)₂NR^{c2}R^{d2}.

40. The compound of claim 1, or a pharmaceutically acceptable salt thereof, wherein Y is CR^{Y} and R^{Y} is independently selected from C₁₋₆ alkyl and OR^{a2}.

41. The compound of claim 1, or a pharmaceutically acceptable salt thereof, wherein R^{a2} is selected from H, C₁₋₆ alkyl, C₁₋₆ haloalkyl, C₆₋₁₀ aryl, C₃₋₇ cycloalkyl, 4-10 membered heterocycloalkyl, C₆₋₁₀ aryl-C₁₋₄ alkyl, C₃₋₇ cycloalkyl-C₁₋₄ alkyl, and 4-10 membered heterocycloalkyl-C₁₋₄ alkyl, wherein said C₁₋₆ alkyl, C₁₋₆ haloalkyl, C₆₋₁₀ aryl, C₃₋₇ cycloalkyl, 4-10 membered heterocycloalkyl, C₆₋₁₀ aryl-C₁₋₄ alkyl, C₃₋₇ cycloalkyl-C₁₋₄ alkyl, and 4-10 membered heterocycloalkyl-C₁₋₄ alkyl are each optionally substituted with 1, 2, 3, 4, or 5 substituents independently selected from C₁₋₄ alkyl, C₁₋₄ haloalkyl, halo, CN, OR^{a3}, C(O)R^{b3}, C(O)OR^{a3} and S(O)₂R^{b3}.

42. The compound of claim 1, or a pharmaceutically acceptable salt thereof, wherein Y is CR^{Y} and R^{Y} is independently selected from NR²R^{d2} and NR^{c2}C(O)R^{b2}.

43. The compound of claim 1, or a pharmaceutically acceptable salt thereof, wherein R^{c2} and R^{d2} are each independently selected from H, C₁₋₆ alkyl, C₁₋₆ haloalkyl, C₆₋₁₀ aryl, C₃₋₇ cycloalkyl, 4-10 membered heterocycloalkyl, C₆₋₁₀ aryl-C₁₋₄ alkyl, C₃₋₇ cycloalkyl-C₁₋₄ alkyl, and 4-10 membered heterocycloalkyl-C₁₋₄ alkyl, wherein said C₁₋₆ alkyl, C₁₋₆ haloalkyl, C₆₋₁₀ aryl, C₃₋₇ cycloalkyl, 4-10 membered heterocycloalkyl, C₆₋₁₀ aryl-C₁₋₄ alkyl, C₃₋₇ cycloalkyl-C₁₋₄ alkyl, and 4-10 membered heterocycloalkyl-C₁₋₄ alkyl are each optionally substituted with 1, 2, 3, 4, or 5 substituents independently selected from C₁₋₄ alkyl, C₁₋₄ haloalkyl, halo, CN, OR^{a3}, C(O)R^{b3}, C(O)OR^{a3} and S(O)₂R^{b3}.

44. The compound of claim 1, or a pharmaceutically acceptable salt thereof, wherein Z is CR^{Z} and R^{Z} is H.

45. The compound of claim 1, or a pharmaceutically acceptable salt thereof, wherein Q is a moiety having Formula II: 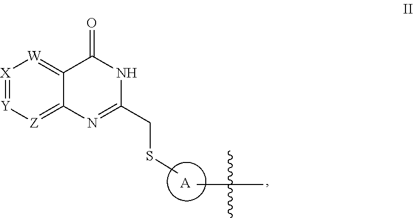
wherein the wavy lines represent the point of attachment to group L¹.

46. The compound of claim 1, or a pharmaceutically acceptable salt thereof, wherein Q is a moiety having Formula IIIA, IIIB, IIIC, IIID, or IIIE: 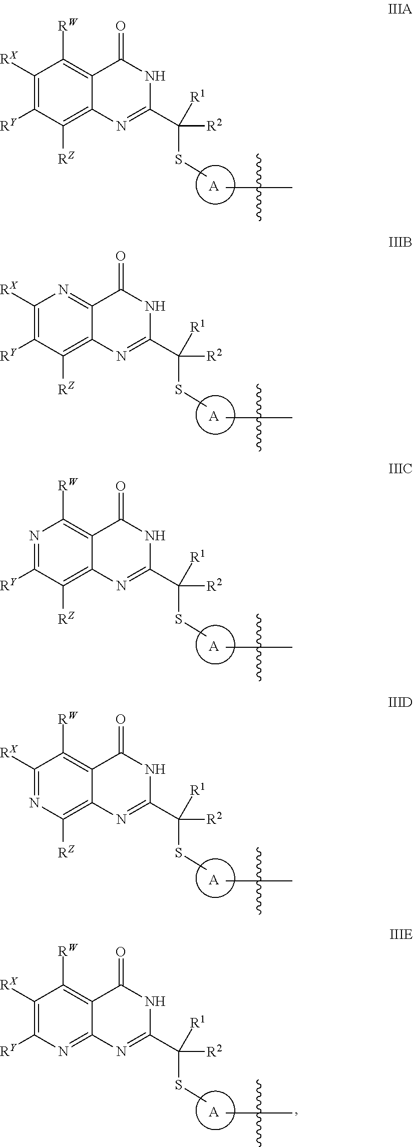
wherein the wavy lines represent the point of attachment to group L¹.

47. The compound of claim 1, or a pharmaceutically acceptable salt thereof, wherein Q is a moiety having Formula IVA or IVB: 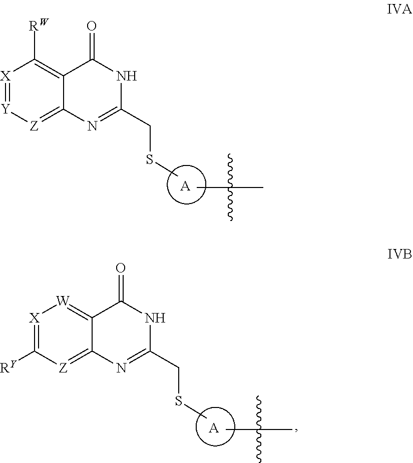
wherein the wavy lines represent the point of attachment to group L¹.

48. The compound of claim 1, having Formula (A2): 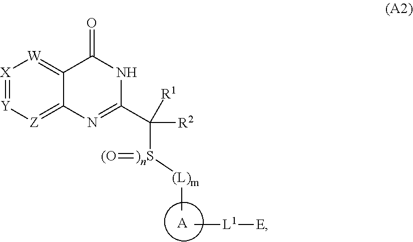
or a pharmaceutically acceptable salt thereof.

49. The compound of claim 1, having Formula (A3): 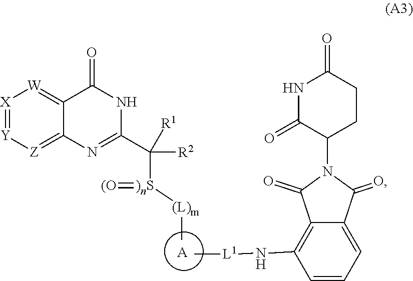
or a pharmaceutically acceptable salt thereof.

50. The compound of claim 1, having Formula (A4): 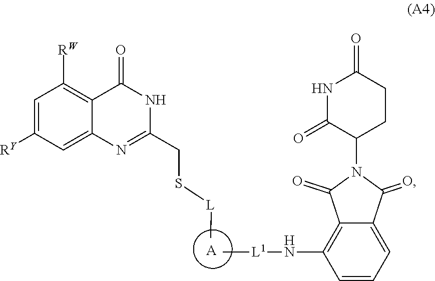
or a pharmaceutically acceptable salt thereof.

51. The compound of claim 1, having Formula (A5): 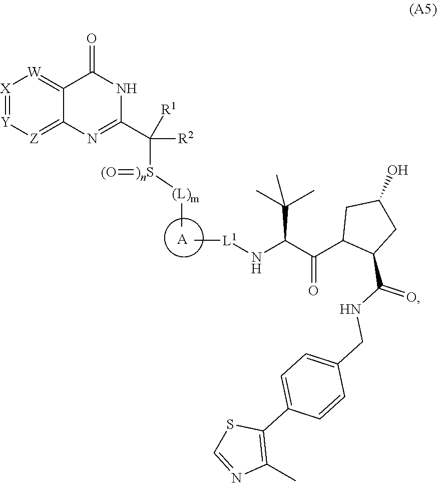
or a pharmaceutically acceptable salt thereof.

52. The compound of claim 1, having Formula (A6): 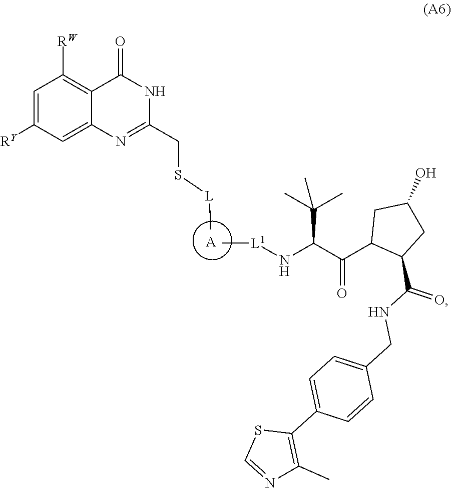
or a pharmaceutically acceptable salt thereof.

53. The compound of claim 1, wherein the compound is selected from the following: 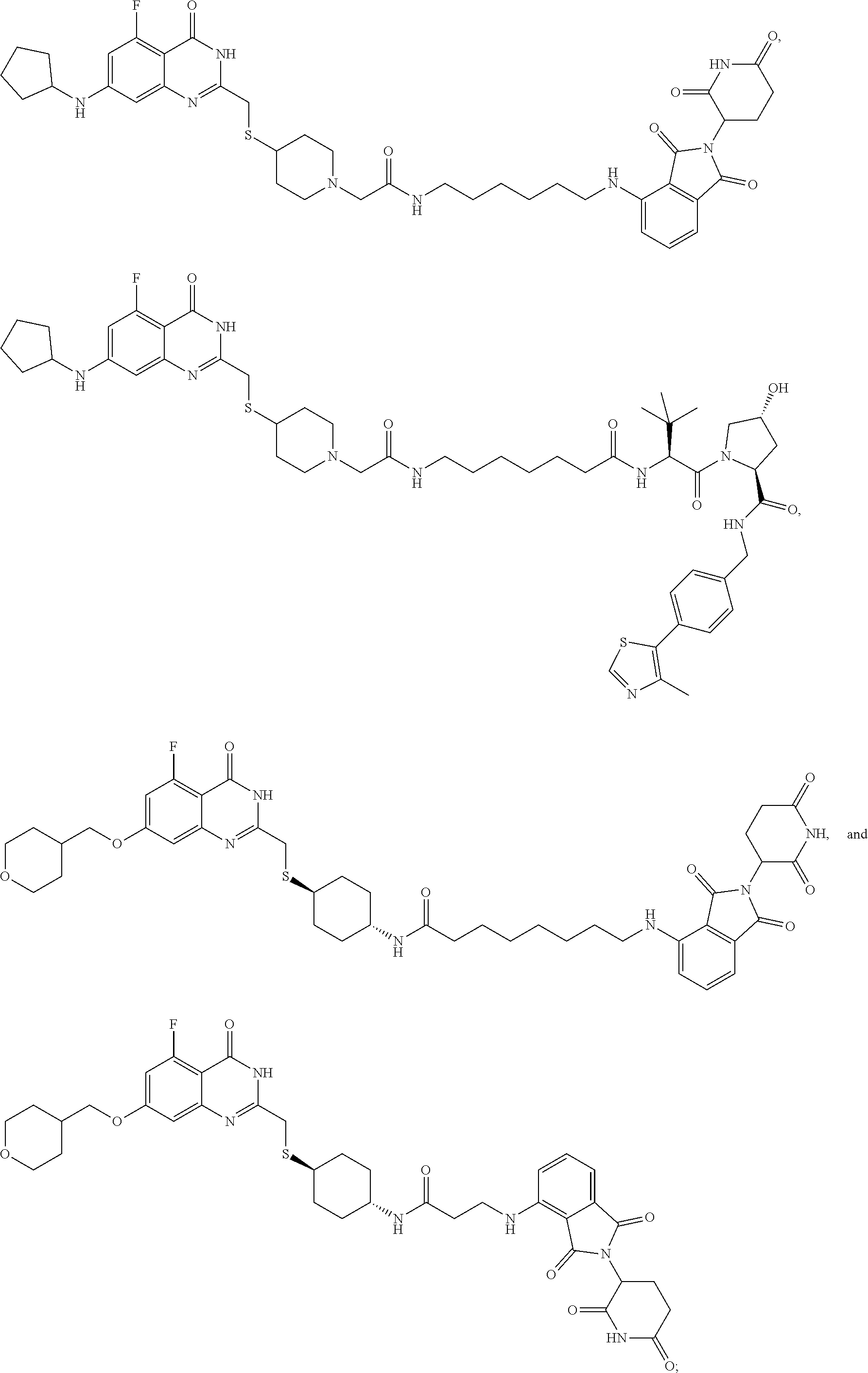 [SMILES: O=C(CN1CCC(SCc2nc3cc(NC4CCCC4)cc(F)c3c(=O)[nH]2)CC1)NCCCCCCNc1cccc2c1C(=O)N(C1CCC(=O)NC1=O)C2=O ; Cc1ncsc1-c1ccc(CNC(=O)[C@@H]2C[C@@H](O)CN2C(=O)[C@@H](NC(=O)CCCCCCNC(=O)CN2CCC(SCc3nc4cc(NC5CCCC5)cc(F)c4c(=O)[nH]3)CC2)C(C)(C)C)cc1 ; O=C1CCC(N2C(=O)c3cccc(NCCCCCCCC(=O)N[C@H]4CC[C@H](SCc5nc6cc(OCC7CCOCC7)cc(F)c6c(=O)[nH]5)CC4)c3C2=O)C(=O)N1 ; "and" ; O=C1CCC(N2C(=O)c3cccc(NCCC(=O)N[C@H]4CC[C@H](SCc5nc6cc(OCC7CCOCC7)cc(F)c6c(=O)[nH]5)CC4)c3C2=O)C(=O)N1]
or a pharmaceutically acceptable salt of any of the aforementioned.

54. A pharmaceutical composition comprising a compound of claim 1, or a pharmaceutically acceptable salt thereof, and at least one pharmaceutically acceptable carrier.

55. A method of degrading PARP14, comprising contacting a compound of claim 1, or a pharmaceutically acceptable salt thereof, with said PARP14.

56. A method of treating cancer in a patient in need of treatment comprising administering to said patient a therapeutically effective amount of a compound of claim 1, or a pharmaceutically acceptable salt thereof.

57. The method of claim 56 wherein said cancer is multiple myeloma, DLBCL, hepatocellular carcinoma, bladder cancer, esophageal cancer, head and neck cancer, kidney cancer, prostate cancer, rectal cancer, stomach cancer, thyroid cancer, uterine cancer, breast cancer, glioma, follicular lymphoma, pancreatic cancer, lung cancer, colon cancer, or melanoma.

58. A method of treating an inflammatory disease in a patient in need of treatment comprising administering to said patient a therapeutically effective amount of a compound of claim 1, or a pharmaceutically acceptable salt thereof.

59. A method of decreasing IL-10 in a cell comprising contacting a compound of claim 1, or a pharmaceutically acceptable salt thereof, with said cell.
